(19) United States
(12) Patent Application Publication
(10) Pub. No.: US 2026/0345954 A1
(43) Pub. Date: Nov. 5, 2026

(54) EXPANDABLE SHEATH FOR INTRODUCING AN ENDOVASCULAR DELIVERY DEVICE INTO A BODY

(71) Applicant: Edwards Lifesciences Corporation, Irvine, CA (US)

(72) Inventors: David Guadalupe Ruiz, Corona, CA (US); Leah Paige Gaffney, Orange, CA (US); Jeong Soo Lee, Yorba Linda, CA (US); Ilan Tamir, Irvine, CA (US); Maxwell Harrison Fine, Costa Mesa, CA (US); Sonny Tran, Westminster, CA (US); Nasser William Saleh, Stockton, CA (US)

(73) Assignee: Edwards Lifesciences Corporation, Irvine, CA (US)

(21) Appl. No.: 19/741,832

(22) Filed: Jul. 14, 2026

Related U.S. Application Data
(63) Continuation of application No. 17/981,699, filed on Nov. 7, 2022, now abandoned.
Application No. 17/981,699 is a continuation of application No. PCT/US2021/031225, filed on May 7, 2021.
(60) Provisional application No. 63/059,764, filed on Jul. 31, 2020.
(60) Provisional application No. 63/021,945, filed on May 8, 2020.

(51) Int. Cl. A61M 25/00; A61M 25/06

(52) U.S. Cl. CPC A61M 25/0023; A61M 25/0009; A61M 25/0045; A61M 25/0662; A61M 2025/0024

(57) ABSTRACT

Aspects of an expandable sheath can be used in conjunction with a catheter assembly to introduce a prosthetic device, such as a heart valve, into a patient. Such aspects can minimize trauma to the vessel by allowing for temporary expansion of a portion of the introducer sheath to accommodate the delivery apparatus, followed by a return to the original diameter once the prosthetic device passes through. Some aspects can include a sheath with inner and outer layers having at least one folded portion of the inner liner. Some aspects include the inner liner having etched, unetched, and otherwise surface-modified portions. Aspects of the present expandable sheath can avoid the need for multiple insertions for the dilation of the vessel, thus offering advantages over prior art introducer sheaths.

### CROSS-REFERENCE TO RELATED APPLICATIONS

This application is a continuation of U.S. Application No. 17/981,699, filed Nov. 7, 2022, which is a continuation of International Application No. PCT/US2021/031225, filed May 7, 2021, which claims the benefit of U.S. Provisional Application No. 63/059,764, filed Jul. 31, 2020, and U.S. Provisional Application No. 63/021,945, filed May 8, 2020, the contents of which are incorporated herein by reference in their entirety.

### FIELD

The present application concerns aspects of a sheath for use with catheter-based technologies for repairing and/or replacing heart valves and delivering a prosthetic device, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss. A conventional introducer sheath typically requires a tubular loader to be inserted through the seals in the housing to provide an unobstructed path through the housing for a valve mounted on a balloon catheter. A conventional loader extends from the proximal end of the introducer sheath and therefore decreases the available working length of the delivery apparatus that can be inserted through the sheath and into the body.

Conventional methods of accessing a vessel, such as a femoral artery, prior to introducing the delivery system include dilating the vessel using multiple dilators or sheaths that progressively increase in diameter. This repeated insertion and vessel dilation can increase the amount of time the procedure takes, as well as the risk of damage to the vessel.

Radially expanding intravascular sheaths have been disclosed. Such sheaths tend to have complex mechanisms, such as ratcheting mechanisms that maintain the shaft or sheath in an expanded configuration once a device with a larger diameter than the sheath's original diameter is introduced.

However, delivery and/or removal of prosthetic devices and other material to or from a patient still poses a significant risk to the patient. Furthermore, accessing the vessel remains a challenge due to the relatively large profile of the delivery system that can cause longitudinal and radial tearing of the vessel during insertion. The delivery system can additionally dislodge calcified plaque within the vessels, posing an additional risk of clots caused by the dislodged plaque.

Accordingly, there remains a need in the art for an improved introducer sheath for endovascular systems used for implanting valves and other prosthetic devices.

### SUMMARY

As disclosed herein, in some aspects, the present expandable sheath can minimize trauma to the vessel by allowing for temporary expansion of a portion of the introducer sheath to accommodate a delivery system, followed by a return to the original diameter once the delivery system passes through. In some aspects, disclosed is a sheath with a smaller profile than that of prior art introducer sheaths. Furthermore, the aspects disclosed herein can reduce the length of time a procedure takes, as well as reduce the risk of a longitudinal or radial vessel tear or plaque dislodgement because only one sheath is required, rather than several different sizes of sheaths. The disclosed aspects of the present expandable sheath can require only a single vessel insertion instead of requiring multiple insertions for the dilation of the vessel.

In one aspect, disclosed herein is a sheath for introducing a prosthetic device comprising an inner liner (layer) and an outer layer. At least a portion of the sheath can be designed or configured to locally expand from a first diameter to a second diameter as the prosthetic device is pushed through a lumen of the sheath, and then at least partially return to the first diameter once the prosthetic device has passed through. In some aspects, an elastic outer cover can also be disposed about the outer layer.

In some aspects, disclosed is a sheath comprising a proximal end and a distal end opposite one another. Some aspects disclosed herein can include a hemostasis valve at or near the proximal end of the sheath. In some aspects, the outer diameter of the sheath can decrease along a gradient from the proximal end to the distal end of the sheath. In yet other aspects, the outer diameter of the sheath can be substantially constant along at least a majority of the length of the sheath.

One aspect described herein is directed to a sheath for delivering a medical device, wherein the sheath has a proximal and a distal end and comprises an expandable inner liner having an inner surface and an outer surface, wherein the inner surface of the expandable inner liner defines a lumen and wherein the inner liner comprises at least one folded portion having an inner portion and outer portion; an outer layer having an inner surface and an outer surface and extending at least partially around the inner liner such that a first portion of the outer surface of the outer layer is positioned adjacent to the inner portion of the at least one folded portion of the inner liner, while a first portion of the inner surface of the outer layer is positioned adjacent to the outer portion of the at least one folded portion of the inner liner; and wherein the inner liner comprises at least one first portion and at least one second portion, wherein an outer surface of the at least one first portion comprises a composition and/or morphology that is substantially different from an outer surface of the at least one second portion; and wherein an outwardly directed radial force from a prosthetic device moving through the inner lumen unfolds the at least one folded portion to allow expansion of the sheath. At least a portion of the sheath is configured to expand to accommodate the prosthetic device.

In some aspects, the at least one first portion of the inner liner is etched. While in other aspects, the at least one second portion of the inner liner is unetched.

In certain aspects, the at least one second portion can be etched and subsequently surface-modified such that a composition of the outer surface of the at least one second portion becomes substantially different from a composition of the outer surface of the at least one first portion.

It is understood that in any of the disclosed herein aspects, the outer layer can be configured such that the overlapping portion overlaps the underlying portion, wherein at least a portion of the folded portion of the inner tubular layer is positioned between the overlapping and underlying portions. In some aspects, at least a portion of the sheath is configured such that a plurality of segments of the sheath each locally expands one at a time from a rest configuration having a first diameter to an expanded configuration having a second diameter that is larger than the first diameter to facilitate passage of the prosthetic device through the lumen of the inner liner. Each segment can have a length defined along the longitudinal axis of the sheath, and each segment of the sheath can be configured to at least partially return to the first diameter once the prosthetic device has passed through. In some aspects, when each segment of the sheath is in the expanded configuration, a length of the folded portion corresponding to the length of the segment at least partially unfolds (e.g., by separating and/or straightening). A length of the overlapping portion corresponding to the length of the segment can be configured to move with respect to the underlying portion when each segment of the sheath expands from the rest configuration to the expanded configuration.

In certain aspects, methods of making a sheath are also disclosed. One method can, for example, and without limitations, include providing a mandrel having a first diameter, providing a first tube having a second diameter, the second diameter being larger than the first diameter, mounting the first tube on the mandrel, gathering excess material of the first tube and folding the excess material to one side to form a folded portion of the inner liner. A second tube can then be provided, and the second tube can be cut to form a coiled layer. An adhesive can be applied to at least a portion of the coiled layer, and the coiled layer can be mounted on the first tube such that the adhesive is positioned between the first tube and the coiled layer. The folded portion can be lifted in order to position a portion of the coiled layer under the folded portion.

In some aspects, some methods can also include applying heat to the first tube, coiled layer, and mandrel so as to thermally fuse the first tube and the coiled layer together. In some other aspects, an elastic outer cover can also be secured to the outer surface of the coiled layer. In still further exemplary and unlimiting aspects, a soft tip portion can be coupled to a distal end of the expandable sheath to facilitate passing the expandable sheath through a patient’s vasculature.

In another aspect, a method of manufacturing a sheath for delivering a medical device can comprise providing an inner liner having an inner surface and an outer surface, wherein the inner surface defines a lumen therethrough, wherein the inner liner comprises at least one first portion and at least one second portion, wherein an outer surface of the at least one first portion comprises a composition and/or morphology that is substantially different from an outer surface of the at least one second portion; and wherein the inner liner comprises at least one folded portion having an inner portion and outer portion; providing an outer layer having an inner surface and an outer surface such that it extends at least partially around the inner liner such that at least a first portion of the outer surface of the outer layer is positioned adjacent to the inner portion of the at least one folded portion of the inner liner, while a first portion of the inner surface of the outer layer is positioned adjacent to the outer portion of the at least one folded portion of the inner liner; and forming an expandable sheath that is configured to expand when an outwardly directed radial force from a prosthetic device moving through the inner lumen unfolds the at least one folded portion.

In still further aspects, the at least one first portion and at least one second portion of the inner liner can be formed by a selective etching. In such exemplary aspects, the selective etching is performed around the circumference, linearly along a length of the sheath, or a combination thereof.

Disclosed herein is an aspect where the at least one first portion can be formed by masking a portion of the inner liner to form a masked portion and then by etching remaining unmasked portions of the inner liner with an etchant. In still further aspects, the methods as disclosed herein can further comprise a step of forming the at least one second portion by unmasking the masked portion after the etching.

In the aspects, as disclosed herein, the methods comprise selective etching. In such exemplary aspects, the selective etching can be done with an etchant. In such aspects, the etchant can comprise a liquid, a fluid, a gas, a plasma, or a combination thereof.

The foregoing and other features and advantages of the disclosure will become more apparent from the following detailed description, which proceeds with reference to the accompanying figures.

### BRIEF DESCRIPTION OF THE DRAWINGS

FIG. 1 is an elevation view of a sheath according to the present disclosure along with an endovascular delivery apparatus for implanting a prosthetic valve.

FIGS. 2A, B, and D are section views of a sheath in various aspects for introducing a prosthetic device into a patient, and FIG. 2C is a perspective view of one component of such a sheath.

FIG. 3 is an elevation view of the sheath shown in FIG. 2.

FIGS. 4A-4B are elevation views of two aspects of a sheath according to the present disclosure, having varying outer diameters.

FIG. 5 illustrates an elevation view of one aspect of a sheath, expanded at a first location to accommodate a delivery system.

FIG. 6 shows an elevation view of the sheath in one aspect, expanded at a second location farther down the sheath.

FIG. 7 shows a section view of another aspect of a sheath that further comprises an outer covering or shell.

FIG. 8 illustrates an elevation view of one aspect of a sheath with an outer covering or shell.

FIG. 9 illustrates a partial elevation view of one aspect of an intermediate tubular layer that can be used to construct a sheath according to the present disclosure.

FIG. 10 illustrates a partial elevation view of another aspect of an intermediate tubular layer having a variable diamond design.

FIG. 11 illustrates a partial elevation view of another aspect of an intermediate tubular layer having a diamond design with spring struts.

FIG. 12 illustrates a partial elevation view of another aspect of an intermediate tubular layer having a diamond design with straight struts.

FIG. 13 illustrates a partial elevation view of another aspect of an intermediate tubular layer having a saw tooth design with spring struts.

FIG. 14 illustrates a partial elevation view of another aspect of an intermediate tubular layer having a saw tooth design with straight struts.

FIG. 15 illustrates a partial elevation view of another aspect of an intermediate tubular layer having a diamond design with straight struts.

FIG. 16 illustrates a partial elevation view of another aspect of an intermediate tubular layer having a helical or spiral design.

FIG. 17 illustrates a partial elevation view of another aspect of an intermediate tubular layer having a diamond design with non-straight struts.

FIG. 18 illustrates a partial elevation view of another aspect of an intermediate tubular layer having an alternative diamond design with non-straight struts.

FIG. 19 illustrates a partial elevation view of another aspect of an intermediate tubular layer having yet another diamond design with non-straight struts.

FIG. 20 illustrates a partial elevation view of another aspect of an intermediate tubular layer having a diamond design with struts.

FIG. 21 illustrates a partial elevation view of another aspect of an intermediate tubular layer having a design similar to that shown in FIG. 20 but with additional struts.

FIG. 22 illustrates a partial elevation view of another aspect of an intermediate tubular layer having a diamond design with spiral struts.

FIG. 23 illustrates a partial elevation view of another aspect of an intermediate tubular layer having a diamond design with adjacent struts.

FIG. 24 illustrates a section view of one aspect of a sheath having a longitudinal notch.

FIG. 25 shows a section view of one aspect of a sheath having a longitudinal cut in the inner liner.

FIG. 26 shows a perspective view of one aspect of a sheath having a plurality of notches or cuts in the outer tubular layer.

FIG. 27A illustrates a section view of one aspect of a sheath, wherein the outer tubular layer contains a longitudinal cut, and the inner liner extends into the gap created by the cut in the outer tubular layer, in an unexpanded configuration; and FIGS. 27B-27E show section views of various aspects of a sheath in the unexpanded configuration.

FIG. 28 shows a section view of the sheath of FIG. 27A in an expanded configuration.

FIGS. 29A-29D show section views of various aspects of a sheath having overlapping sections.

FIG. 30 illustrates a block diagram of one aspect of a method of making a sheath according to the present disclosure.

FIG. 31 illustrates a block diagram of another aspect of a method of making a sheath according to the present disclosure.

FIGS. 32A-32H illustrates section or elevation views of various method steps of the methods shown in FIGS. 30-31.

FIG. 33 illustrates a plan view of one aspect of a sheath having a partial slit or score line.

FIG. 34 illustrates a plan view of another aspect of a sheath having a partial slit or score line.

FIG. 35 is an elevation view of an expandable sheath according to the present disclosure and a representative housing.

FIG. 36 is an enlarged cutaway view of the distal end of the sheath of FIG. 35.

FIG. 37 is a section view of the distal end of the sheath of FIG. 35, taken along line 37-37 in FIG. 36.

FIG. 38 is a section view of a proximal section of the sheath of FIG. 35, taken along line 38-38 in FIG. 35.

FIG. 39 is a section view of the sheath of FIG. 35 in a rest (unexpanded) configuration, taken along line 39-39 in FIG. 35.

FIG. 40 is the section view of the sheath of FIG. 39, in an expanded configuration.

FIG. 41 shows an elevation view of an expandable sheath having an elastic outer cover, according to another aspect.

FIG. 42 illustrates a section view of the sheath of FIG. 41, taken along line 42-42 in FIG. 41.

FIG. 43 illustrates a section view of the sheath shown in FIG. 42, in an expanded configuration.

FIG. 44 illustrates a section view of another aspect of an expandable sheath.

FIG. 45 shows an expanded configuration of the sheath of FIG. 44.

FIG. 46 illustrates a section view of another aspect of an expandable sheath.

FIG. 47 shows an expanded configuration of the sheath of FIG. 46.

FIG. 48 illustrates a section view of another aspect of an expandable sheath according to the present disclosure.

FIG. 49 illustrates a section view of another aspect of an expandable sheath.

FIG. 50 is a section view of an example sheath in an unexpanded configuration.

FIG. 51 is a section view of the sheath of FIG. 50 in an expanded configuration.

FIG. 52 is a section view of the sheath of FIG. 50, including an outer jacket.

FIG. 53 is a section view of the sheath of FIG. 35 in a rest (unexpanded) configuration, including an outer jacket, taken along line 39-39 in FIG. 35.

FIG. 54 is a section view of the sheath of FIG. 53 in a rest (unexpanded) configuration, taken along line 39-39 in FIG. 35.

FIG. 55 is a section view of the sheath of FIG. 54, in an expanded configuration.

FIG. 56 is a section view of the sheath of FIG. 54 in a rest (unexpanded) configuration, including a lubricant between the outer layer and the outer jacket.

FIG. 57 is a section view of the sheath of FIG. 54 in a rest (unexpanded) configuration, including a lubricant and a bonding strip.

FIG. 58 is a bottom perspective view of the sheath of FIG. 57.

FIG. 59 is a bottom perspective view of the sheath of FIG. 57.

FIG. 60 is a top perspective view of the sheath of FIG. 54.

FIGS. 61A-B is a schematic view of an inner liner having first and second portions as disclosed in one aspect.

FIGS. 62A-B is a schematic view of an inner liner having first and second portions as disclosed in one aspect.

FIGS. 63A-B is a schematic view of an inner liner having first and second portions as disclosed in one aspect.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and the claims, the singular forms “a,” “an,” and “the” include the plural forms unless the context clearly dictates otherwise. Thus, for example, reference to a “polymer” includes aspects having two or more such polymers unless the context clearly indicates otherwise.

It is appreciated that certain features of the disclosure, which are, for clarity, described in the context of separate aspects, can also be provided in combination or as a single aspect. Conversely, various features of the disclosure, which are, for brevity, described in the context of a single aspect, can also be provided separately or in any suitable combination.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and the claims, the term “comprising” can include the aspects “consisting of” and “consisting essentially of.” Additionally, the term “includes” means “comprises.”

For the terms “for example” and “such as,” and grammatical equivalences thereof, the phrase “and without limitation” is understood to follow unless explicitly stated otherwise.

References in the specification and concluding claims to parts by weight of a particular element or component in a composition or article denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5 and are present in such ratio regardless of whether additional components are contained in the composition.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “composition” is intended to encompass a product comprising the specified ingredients in the specified amounts, as well as any product which results, directly or indirectly, from a combination of the specified ingredients in the specified amounts.

A weight percent of a component, unless specifically stated to the contrary, is based on the total weight of the formulation or composition in which the component is included.

As used herein, the term “substantially” means that the subsequently described event or circumstance completely occurs or that the subsequently described event or circumstance generally, typically, or approximately occurs.

As used herein, the term “substantially,” when used in reference to a composition, refers to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” in, for example, the context “substantially free” refers to a composition having less than about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component.

Further, the terms “coupled” and “associated” generally mean electrically, electromagnetically, and/or physically (e.g., mechanically or chemically) coupled or linked and does not exclude the presence of intermediate elements between the coupled or associated items.

It will be understood that, although the terms “first,” “second,” etc., may be used herein to describe various elements, components, regions, layers and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of exemplary aspects.

It is understood that the terms “layer” and “liner” can be used interchangeably. For example, the aspects describing an “inner liner” also include aspects describing an “inner layer.” Similarly, the aspects describing an “outer layer” also include aspects describing an “outer liner.”.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “atraumatic” is commonly known in the art and refers to a device or a procedure that minimized tissue injury.

Although the operations of exemplary aspects of the disclosed method can be described in a particular, sequential order for convenient presentation, it should be understood that disclosed aspects can encompass an order of operations other than the particular, sequential order disclosed. For example, operations described sequentially may, in some cases, be rearranged or performed concurrently. Further, descriptions and disclosures provided in association with one particular aspect are not limited to that aspect and may be applied to any aspect disclosed.

While aspects of the present disclosure can be described and claimed in a particular statutory class, such as the system statutory class, this is for convenience only, and one of ordinary skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present disclosure may be understood more readily by reference to the following detailed description of various aspects of the disclosure and the examples included therein and to the Figures and their previous and following description.

The present disclosure may be understood more readily by reference to the following detailed description of various aspects of the disclosure and the examples included therein and to the Figures and their previous and following description.

Disclosed aspects of an expandable sheath can minimize trauma to the vessel by allowing for temporary expansion of a portion of the introducer sheath to accommodate the delivery system, followed by a return to the original diameter once the device passes through. In some aspects, disclosed herein is a sheath with a smaller profile (e.g., a smaller diameter in the rest configuration) than that of prior art introducer sheaths. Furthermore, the disclosed herein aspects can reduce the length of time a procedure takes, as well as reduce the risk of a longitudinal or radial vessel tear or plaque dislodgement because only one sheath is required, rather than several different sizes of sheaths. Aspects of the present expandable sheath can avoid the need for multiple insertions for the dilation of the vessel. Such expandable sheaths can be useful for many types of minimally invasive surgery, such as any surgery requiring introduction of an apparatus into a subject's vessel. For example, the sheath can be used to introduce other types of delivery apparatus for placing various types of intraluminal devices (e.g., stents, prosthetic heart valves, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

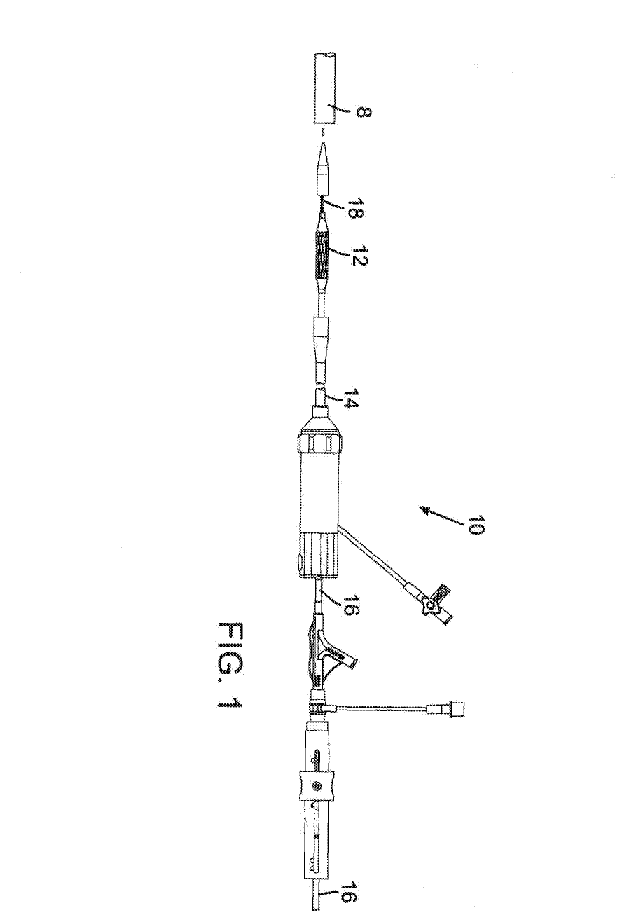

FIG. 1 illustrates a sheath 8 according to the present disclosure, in use with a representative delivery apparatus 10, for delivering a prosthetic device 12, such as a tissue heart valve to a patient. The apparatus 10 can include a steerable guide catheter 14 (also referred to as a flex catheter), a balloon catheter 16 extending through the guide catheter 14, and a nose catheter 18 extending through the balloon catheter 16. The guide catheter 14, the balloon catheter 16, and the nose catheter 18 in the illustrated aspect are adapted to slide longitudinally relative to each other to facilitate delivery and positioning of the valve 12 at an implantation site in a patient's body as described in detail below. Generally, sheath 8 is inserted into a vessel, such as the transfemoral vessel, passing through the skin of the patient, such that the distal end of the sheath 8 is inserted into the vessel. Sheath 8 can include a hemostasis valve at the opposite, proximal end of the sheath. The delivery apparatus 10 can be inserted into the sheath 8, and the prosthetic device 12 can then be delivered and implanted within the patient.

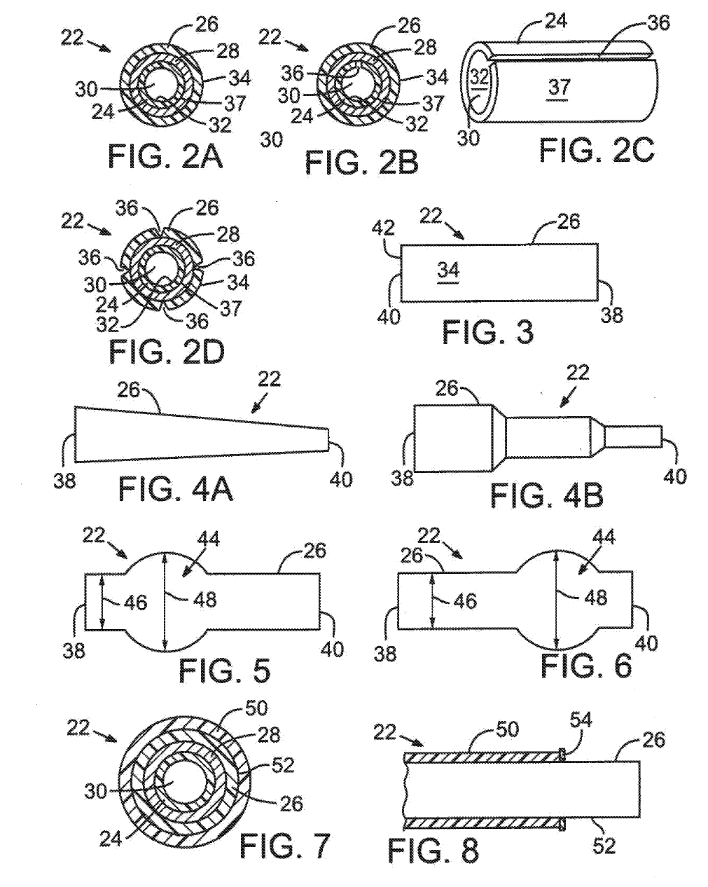

FIGS. 2A, 2B and 2D show section views of exemplary aspects of a sheath 22 for use with a delivery apparatus such as that shown in FIG. 1. Example expandable sheaths are also disclosed in U.S. patent application Ser. No. 12/249,867, filed Oct. 10, 2008 (now U.S. Pat. No. 8,690,936), U.S. patent application Ser. No. 13/312,739, filed Dec.6, 2011 (now U.S. Pat. No. 8,790,387), U.S. patent application Ser. No. 14/248,120 filed on Apr. 8, 2014 (now U.S. Pat. No. 9,301,840), U.S. patent application Ser. No. 14/324,894, filed Jul. 7, 2014 (now U.S. Pat. No. 9,301,841), U.S. patent application Ser. No. 15/057,953, filed Mar.1, 2016 (now U.S. Pat. No. 9,987,134), U.S. patent application Ser. No. 15/997,587, filed Jun. 4, 2018, U.S. patent application Ser. No. 16/149,953, filed on Oct. 2, 2018 (now U.S. Pat. No. 10,524,905), U.S. patent application Ser. No. 16/149,956, filed on Oct. 2, 2018 (now U.S. Pat. No. 10,517,720), U.S. patent application Ser. No. 16/149,960, filed on Oct. 2, 2018 (now U.S. Pat. No. 10,524,906, and U.S. patent application Ser. No. 16/149,969, filed on Oct. 2, 2018 (now U.S. Pat. No. 10,524,907), the disclosures of which are herein incorporated by reference.

FIG. 2C shows a perspective view of one aspect of an inner liner 24 for use with the sheath 22. Sheath 22 includes an inner liner, such as inner polymeric tubular layer 24, an outer layer, such as outer polymeric tubular layer 26, and an intermediate tubular layer 28 disposed between the inner and outer polymeric tubular layers 24, 26. The sheath 22 defines a lumen 30 through which a delivery apparatus can travel into a patient's vessel in order to deliver, remove, repair, and/or replace a prosthetic device. Such introducer sheaths 22 can also be useful for other types of minimally invasive surgery, such as any surgery requiring introduction of an apparatus into a subject's vessel. For example, the sheath 22 also can be used to introduce other types of delivery apparatus for placing various types of intraluminal devices (e.g., stents, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

The outer polymeric tubular layer 26 and the inner polymeric tubular layer 24 can comprise, for example, PTFE (e.g., Teflon®), polyimide, PEEK, polyurethane, nylon, polyethylene, polyamide, polyether block amides (e.g., PEBAX®) , polyether block ester copolymer, polyesters, fluoropolymers, polyvinyl chloride, thermoset silicone, latex, poly-isoprene rubbers, polyolefin, other medical grade polymers, or combinations thereof. The intermediate tubular layer 28 can comprise a shape memory alloy such as Nitinol and/or stainless steel, cobalt chromium, spectra fiber, polyethylene fiber, aramid fiber, or combinations thereof.

The inner polymeric tubular layer 24 can advantageously be provided with a low coefficient of friction on its inner surface. For example, the inner polymeric tubular layer 24 can have a coefficient of friction of less than about 0.1. Some aspects of a sheath 22 can include a lubricious liner on the inner surface 32 of the inner polymeric tubular layer 24. Such a liner can facilitate passage of a delivery apparatus through the lumen 30 of the sheath 22. Examples of suitable lubricious liners include materials that can reduce the coefficient of friction of the inner polymeric tubular layer 24, such as PTFE, polyethylene, polyvinylidene fluoride, and combinations thereof. Suitable materials for a lubricious liner also include other materials desirably having a coefficient of friction of about 0.1 or less.

The inner diameter of the intermediate tubular layer 28 varies depending on the application and size of the delivery apparatus and prosthetic device. In some aspects, the inner diameter ranges from about 0.005 inches to about 0.400 inches. The thickness of the intermediate tubular layer 28 can be varied depending on the desired amount of radial expansion, as well as the strength required. For example, the thickness of the intermediate tubular layer 28 can be from about 0.002 inches to about 0.025 inches. The thicknesses of the inner polymeric tubular layer 24 and the outer polymeric tubular layer 26 can also be varied depending on the particular application of the sheath 22. In some aspects, the thickness of the inner polymeric tubular layer 24 ranges from about 0.0005 inches to about 0.010 inches, and in one particular aspect, the thickness is about 0.002 inches. Outer polymeric tubular layer 26 can have a thickness of from about 0.002 inches to about 0.015 inches, and in one particular aspect, the outer polymeric tubular layer 26 has a thickness of about 0.010 inches.

The hardness of each layer of the sheath 22 can also be varied depending on the particular application and desired properties of the sheath 22. In some aspects, the outer polymeric tubular layer 26 has a Shore hardness of from about 25 Durometer to about 75 Durometer.

Additionally, some aspects of a sheath 22 can include an exterior hydrophilic coating on the outer surface 34 of the outer polymeric tubular layer 26. Such a hydrophilic coating can facilitate insertion of the sheath 22 into a patient's vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings, are also suitable for use with the sheath 22.

In some aspects, the outer surface 34 of the outer polymeric tubular layer 26 can be modified. For example, surface modifications such as plasma etching can be performed on the outer surface 34. Similarly, other surfaces, both outer and inner, can be surface modified according to certain aspects and desired application. In some aspects, surface modification can improve adhesion between the layers in the areas of the modification.

The sheath 22 also can have at least one radiopaque filler or marker. The radiopaque filler or marker can be associated with the outer surface 34 of the outer polymeric tubular layer 26. Alternatively, the radiopaque filler or marker can be embedded or blended within the outer polymeric tubular layer 24. Similarly, the radiopaque filler or marker can be associated with a surface of the inner polymeric tubular layer 24 or the intermediate tubular layer 28 or embedded within either or both of those layers.

Suitable materials for use as a radiopaque filler or marker include, for example, barium sulfite, bismuth trioxide, titanium dioxide, bismuth subcarbonate, or combinations thereof. The radiopaque filler can be mixed with or embedded in the material used to form the outer polymeric tubular layer 26 and can comprise from about 5% to about 45% by weight of the outer polymeric tubular layer. More or less radiopaque material can be used in some aspects, depending on the particular application.

In some aspects, the inner polymeric tubular layer 24 can comprise a substantially uniform cylindrical tube. In alternative aspects, the inner polymeric tubular layer 24 can have at least one section of discontinuity along its longitudinal axis to facilitate radial expansion of the inner polymeric tubular layer 24. For example, the inner polymeric tubular layer 24 can be provided with one or more longitudinal notches and/or cuts 36 extending along at least a portion of the length of the sheath 22. Such notches or cuts 36 can facilitate radial expansion of the inner polymeric tubular layer 24, thus accommodating passage of a delivery apparatus or other device. Such notches and/or cuts 36 can be provided near the inner surface 32, near the outer surface 37, and/or substantially through the entire thickness of the inner polymeric layer 24. In aspects with a plurality of notches and/or cuts 36, such notches and/or cuts 36 can be positioned such that they are substantially equally spaced from one another circumferentially around the inner polymeric layer 24. Alternatively, notches and cuts 36 can be spaced randomly in relation to one another or in any other desired pattern. Some or all of any provided notches and/or cuts 36 can extend longitudinally along substantially the entire length of the sheath 22. Alternatively, some or all of any provided notches and/or cuts 36 can extend longitudinally only along a portion of the length of the sheath 22.

As shown in FIGS. 2B and 2C (which illustrates only the inner polymeric tubular layer 24), in some aspects, the inner polymeric tubular layer 24 contains at least one notch or cut 36 that extends longitudinally and parallel to an axis defined by the lumen 30, extending substantially the entire length of the sheath 22. Thus, upon introduction of a delivery apparatus, the inner polymeric tubular layer 24 can split open along the notch and/or cut 36 and expand, thus accommodating the delivery apparatus.

Additionally or alternatively, as shown in FIG. 2D, the outer polymeric tubular layer 26 can comprise one or more notches and/or cuts 36. Notches and/or cuts 36, in some aspects, do not extend through the entire thickness of the outer tubular layer 26. The notches and/or cuts 36 can be separable upon radial expansion of the sheath 22. The outer polymeric tubular layer 26 can be retractable longitudinally or able to be pulled back away from the intermediate tubular layer 28 and the inner polymeric tubular layer 24. In aspects with a retractable outer polymeric tubular layer 26, the outer polymeric tubular layer 26 can be retracted to accommodate or facilitate passage of a delivery apparatus through the lumen 30 and then can be replaced to its original position on the sheath 22.

FIG. 3 illustrates an elevation view of the sheath 22 shown in FIG. 2A. In this view, only the outer polymeric tubular layer 26 is visible. The sheath 22 comprises a proximal end 38 and a distal end 40 opposite the proximal end 38. The sheath 22 can include a hemostasis valve inside the lumen of the sheath 22, at or near the proximal end 38 of the sheath 22.

Additionally, the sheath 22 can comprise a soft tip 42 at the distal end 40 of the sheath 22. Such a soft tip 42 can be provided with a lower hardness than the other portions of the sheath 22. In some aspects, the soft tip 42 can have a Shore hardness from about 25 D to about 40 D.

As shown in FIG. 3, the unexpanded original outer diameter of the sheath 22 can be substantially constant across the length of the sheath 22, substantially from the proximal end 38 to the distal end 40. In alternative aspects, such as the ones illustrated in FIGS. 4A-4B, the original unexpanded outer diameter of the sheath 22 can decrease from the proximal end 38 to the distal end 40. As shown in the aspect in FIG. 4A, the original unexpanded outer diameter can decrease along a gradient, from the proximal end 38 to the distal end 40. In alternative aspects, such as the one shown in FIG. 4B, the original unexpanded outer diameter of sheath 22, can incrementally step down along the length of the sheath 22, wherein the largest original unexpanded outer diameter is near the proximal end 38, and the smallest original unexpanded outer diameter is near the distal end 40 of the sheath 22.

As shown in FIGS. 5-6, the sheath 22 can be designed to locally expand as the prosthetic device is passed through the lumen of the sheath 22 and then substantially return to its original shape once the prosthetic device has passed through that portion of the sheath 22. For example, FIG. 5 illustrates a sheath 22 having a localized bulge 44, representative of a device being passed through the internal lumen of the sheath 22. FIG. 5 shows the device close to the proximal end 38 of the sheath 22, close to the area where the device is introduced into the sheath 22. FIG. 6 shows the sheath 22 of FIG. 5, with the device having progressed further along the sheath 22. The localized bulge 44 is now closer to the distal end 40 of the sheath 22, and thus, is about to be introduced to a patient's vessel. As evident from FIGS. 5 and 6, once the localized bulge associated with the device has passed through a portion of the lumen of the sheath 22, that portion of the sheath 22 can automatically return to its original shape and size, at least in part due to the materials and structure of the sheath 22.

The sheath 22 has an unexpanded inner diameter equal to the inner diameter of the inner polymeric tubular layer (not visible in FIGS. 5-6) and an unexpanded outer diameter 46 equal to the outer diameter of the outer polymeric tubular layer 26. The sheath 22 is designed to be expanded to an expanded inner diameter and an expanded outer diameter 48, which are larger than the unexpanded inner diameter and the unexpanded outer diameter 46, respectively. In one representative aspect, the unexpanded inner diameter is about 16 Fr, and the unexpanded outer diameter 46 is about 19 Fr, while the expanded inner diameter is about 26 Fr, and the expanded outer diameter 48 is about 29 Fr. Different sheaths 22 can be provided with different expanded and unexpanded inner and outer diameters, depending on the size requirements of the delivery apparatus for various applications. Additionally, some aspects can provide more or less expansion depending on the particular design parameters, the materials, and/or configurations used.

In some aspects of a sheath according to the present disclosure, and as shown in section in FIG. 7 and in elevation in FIG. 8, the sheath 22 can additionally comprise an outer covering, such as outer polymeric covering 50, disposed on the outer surface 52 of the outer polymeric tubular layer 26. The outer polymeric covering 50 can provide a protective covering for the underlying sheath 22. In some aspects, the outer polymeric covering 50 can contain a self-expandable sheath in a crimped or constrained state and then release the self-expandable sheath upon removal of the outer polymeric covering 50. For example, in some aspects of a self-expandable sheath, the intermediate layer 28 can comprise Nitinol and/or other shape memory alloys, and the intermediate layer 28 can be crimped or radially compressed to a reduced diameter within the outer polymeric tubular layer 26 and the outer polymeric covering 50. Once the self-expandable sheath is at least partially inserted into a patient's vessel, the outer polymeric covering 50 can be slid back, peeled away, or otherwise at least partially removed from the sheath. To facilitate removal of the outer polymeric covering 50, a portion of the outer polymeric covering 50 can remain outside the patient's vessel, and that portion can be pulled back or removed from the sheath to allow the sheath to expand. In some aspects, substantially the entire outer polymeric covering 50 can be inserted, along with the sheath, into a patient's vessel. In these aspects, an external mechanism attached to the outer polymeric covering 50 can be provided, such that the outer polymeric covering can be at least partially removed from the sheath once the sheath is inserted into a patient's vessel.

Once no longer constrained by the outer polymeric covering 50, the radially compressed intermediate layer 28 can self-expand, causing expansion of the sheath along the length of the intermediate layer 28. In some aspects, portions of the sheath can radially collapse, at least partially returning to the original crimped state, as the sheath is being withdrawn from the vessel after completion of the surgical procedure. In some aspects, such collapse can be facilitated and/or encouraged by an additional device or layer that, in some aspects, can be mounted onto a portion of the sheath prior to the sheath's insertion into the vessel.

The outer polymeric covering 50, in some aspects, is not adhered to the other layers of the sheath 22. For example, the outer polymeric covering 50 may be slidable with respect to the underlying sheath, such that it can be easily removed or retracted from its initial position on the sheath 22.

As seen in FIG. 8, the outer polymeric covering 50 can include one or more peel tabs 54 to facilitate manual removal of the outer polymeric covering 50. The outer polymeric covering 50 can be automatically or manually retractable and/or splittable to facilitate radial expansion of the sheath 22. Peel tabs 54 can be located approximately 90 degrees from any cut or notch present in the outer polymeric covering 50, and approximately 180 degrees offset from one another. In alternative aspects, the peel tabs 54 can extend substantially around the circumference of the outer polymeric covering 50, thus resulting in a single circular peel tab 54.

Suitable materials for the outer polymeric covering 50 are similar to those materials suitable for the inner polymeric tubular layer and the outer polymeric tubular layer and can include PTFE and/or high density polyethylene.

Turning now to the intermediate tubular layer 28, several different configurations are possible. The intermediate tubular layer 28 is generally a thin, hollow, substantially cylindrical tube comprising an arrangement, pattern, structure, or configuration of wires or struts, however, other geometries can also be used. The intermediate tubular layer 28 can extend along substantially the entire length of the sheath 22, or alternatively, can extend only along a portion of the length of sheath 22. Suitable wires can be round, ranging from about 0.0005 inches thick to about 0.10 inches thick, or flat, ranging from about 0.0005 inches×0.003 inches to about 0.003 inches×0.007 inches. However, other geometries and sizes are also suitable for certain aspects. If a braided wire is used, the braid density can be varied. Some aspects have a braid density of about thirty picks per inch to about eighty picks per inch and can include up to thirty-two wires in various braid patterns.

One representative aspect of an intermediate tubular layer comprises a braided Nitinol composite, which is at least partially encapsulated by an inner polymeric tubular member and an outer polymeric tubular member disposed on inner and outer surfaces of the intermediate tubular layer, respectively. Such encapsulation by polymeric layers can be accomplished by, for example, fusing the polymeric layers to the intermediate tubular layer or dip coating the intermediate tubular layer. In some aspects, an inner polymeric tubular member, an intermediate tubular layer, and an outer polymeric tubular layer can be arranged on a mandrel, and the layers can then be thermally fused or melted into one another by placing the assembly in an oven or otherwise heating it. The mandrel can then be removed from the resulting sheath. In other aspects, dip coating can be used to apply an inner polymeric tubular member to the surface of a mandrel. The intermediate tubular layer can then be applied, and the inner polymeric tubular member allowed to cure. The assembly can then be dip coated again, such as to apply a thin coating of, for example, polyurethane, which will become the outer polymeric tubular member of the sheath. The sheath can then be removed from the mandrel.

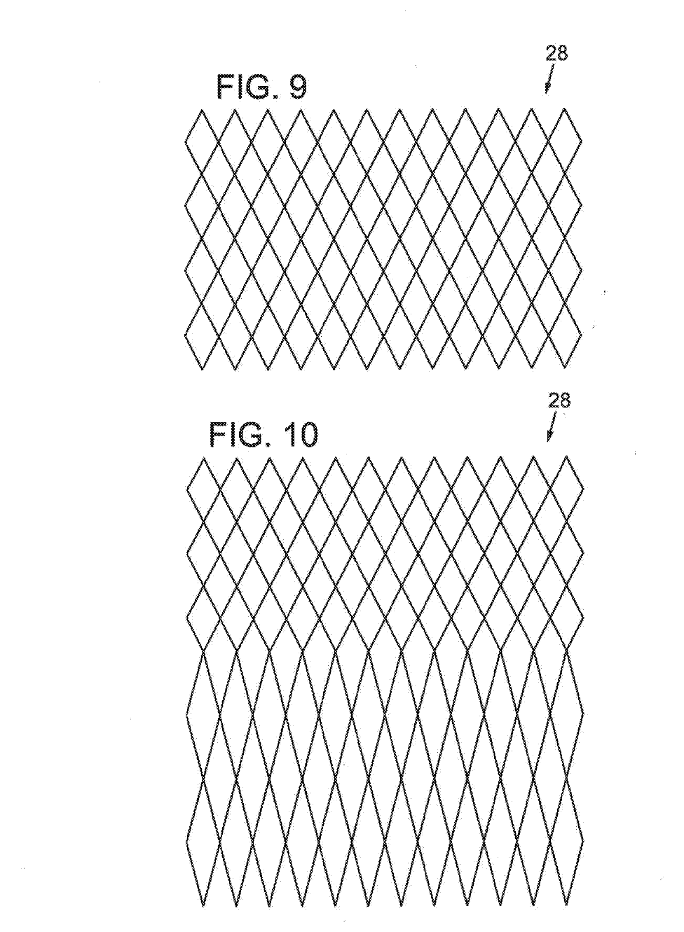

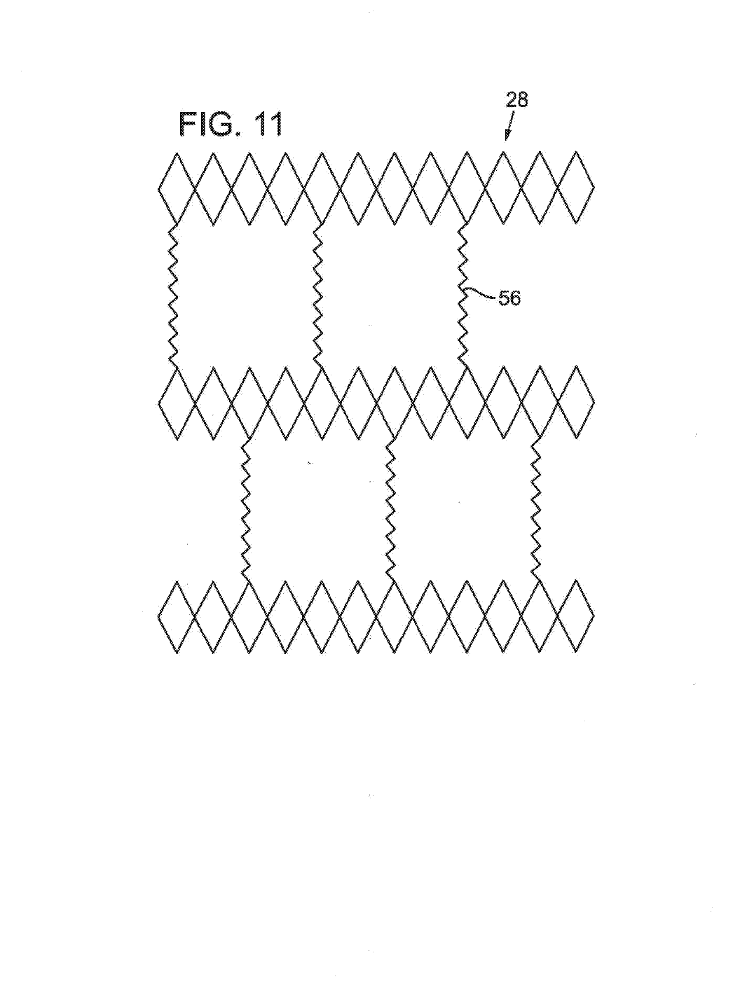

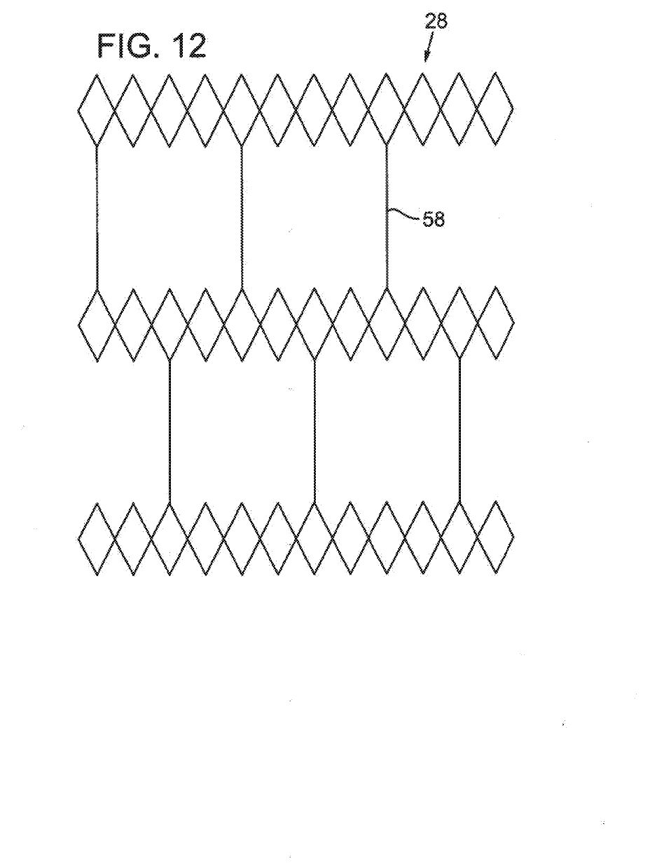

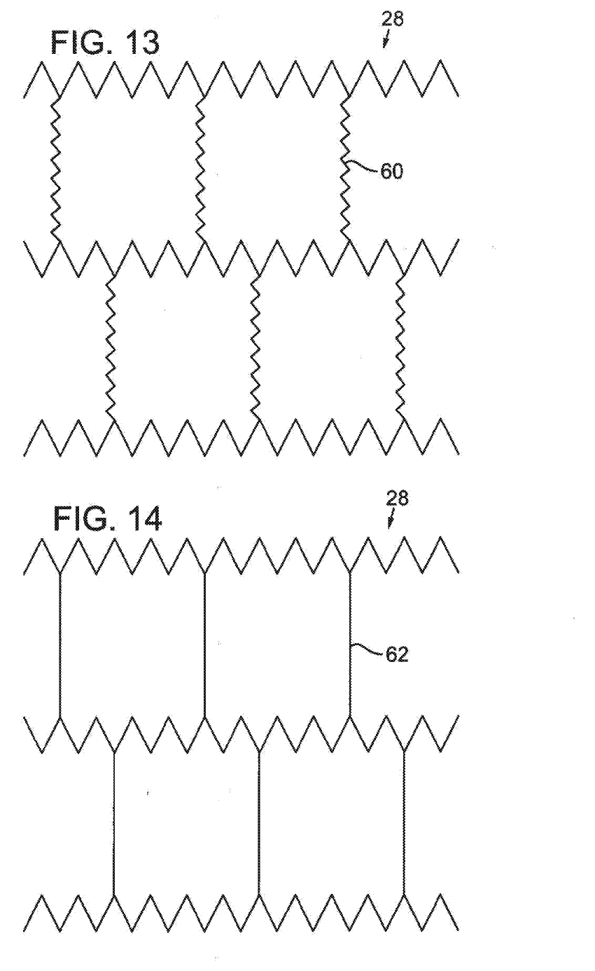

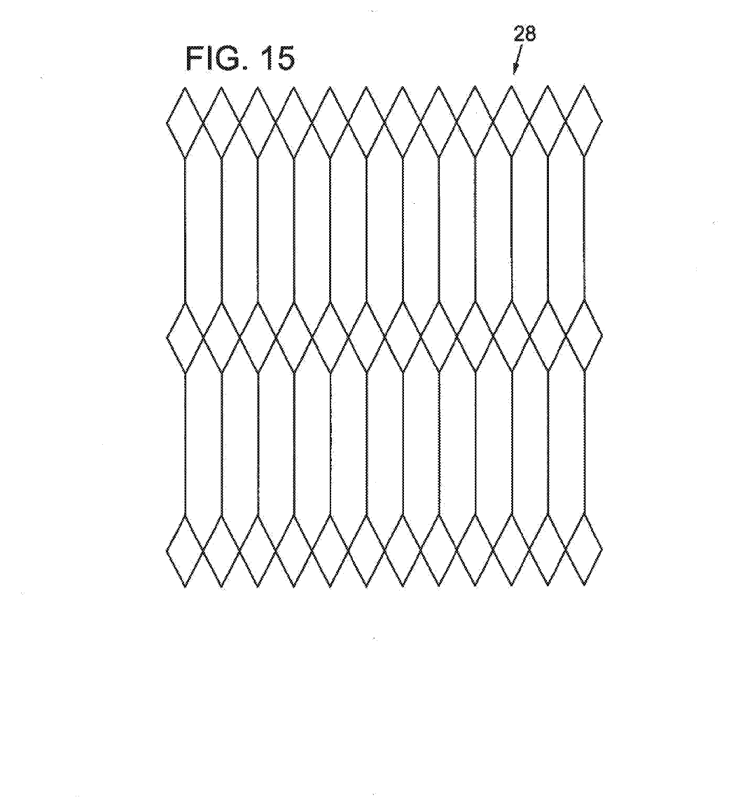

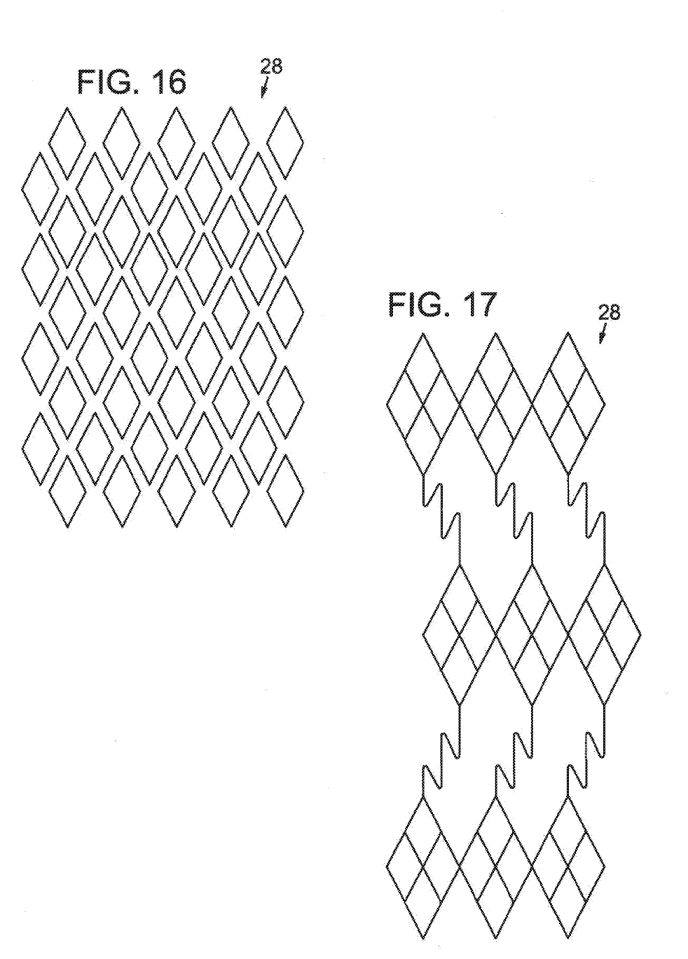

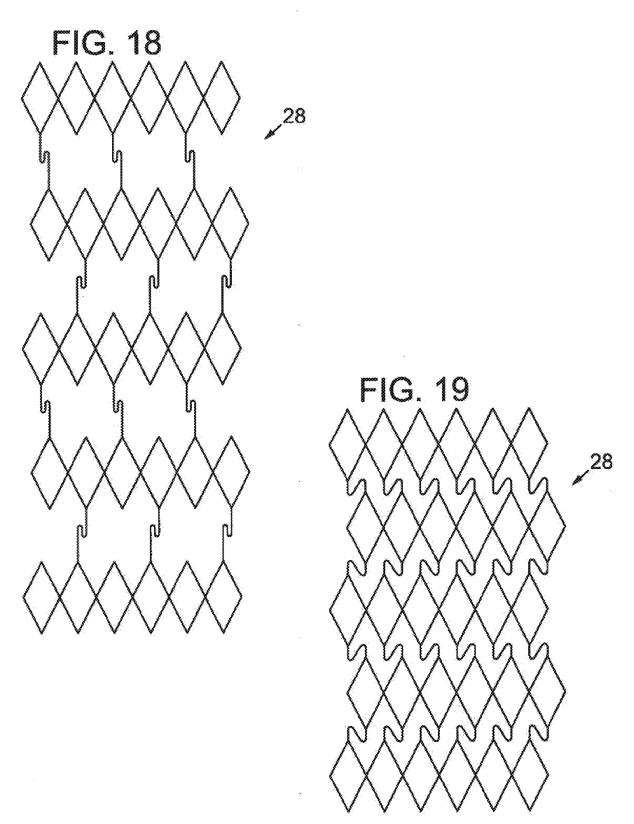

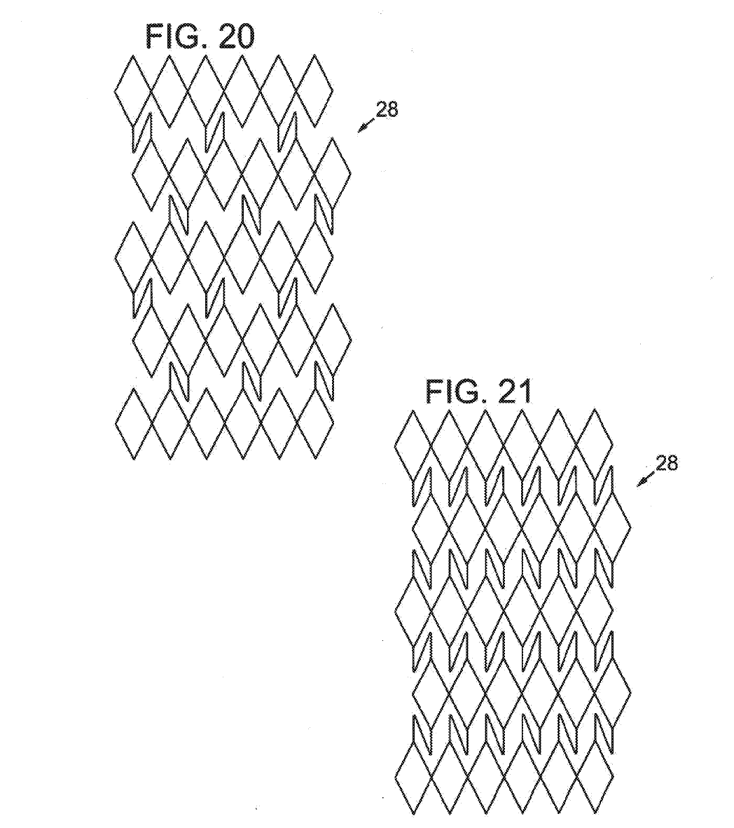

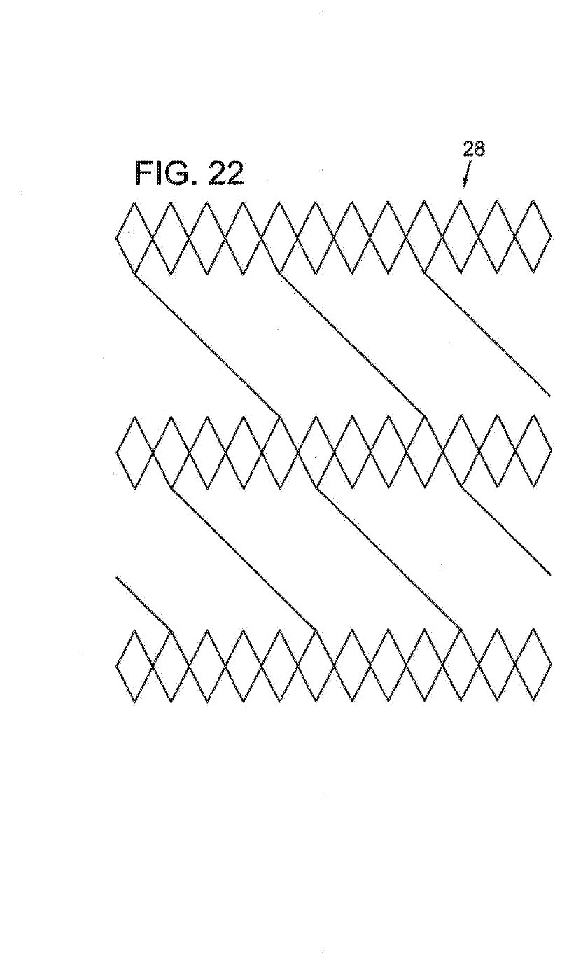

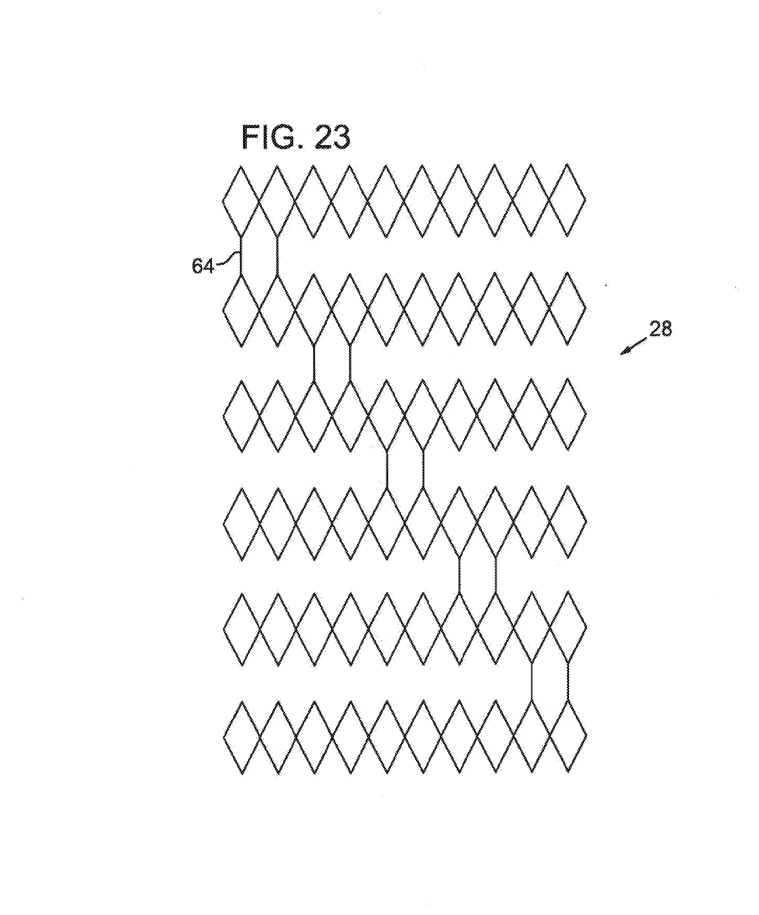

Additionally, the intermediate tubular layer 28 can be, for example, braided or laser cut to form a pattern or structure, such that the intermediate tubular layer 28 is amenable to radial expansion. FIGS. 9-23 illustrate partial elevation views of various structures for the intermediate tubular layer. Some illustrated structures, such as those shown in FIGS. 11-14 and 23, include at least one discontinuity. For example, the struts 56, 58, 60, 62, 64, shown in FIGS. 11, 12, 13, 14 and 23 respectively, resulting in a discontinuous intermediate tubular layer 28 in that the struts 56, 58, 60, 62, 64 separate adjacent sections of the intermediate tubular layer 28 from each other, where the sections are spaced apart from each other along a longitudinal axis parallel to the lumen of the sheath. Thus, the structure of the intermediate tubular layer 28 can vary from section to section, changing along the length of the sheath.

The structures shown in FIGS. 9-23 are not necessarily drawn to scale. Components and elements of the structures can be used alone or in combination within a single intermediate tubular layer 28. The scope of the intermediate tubular layer 28 is not meant to be limited to these particular structures; they are merely exemplary aspects.

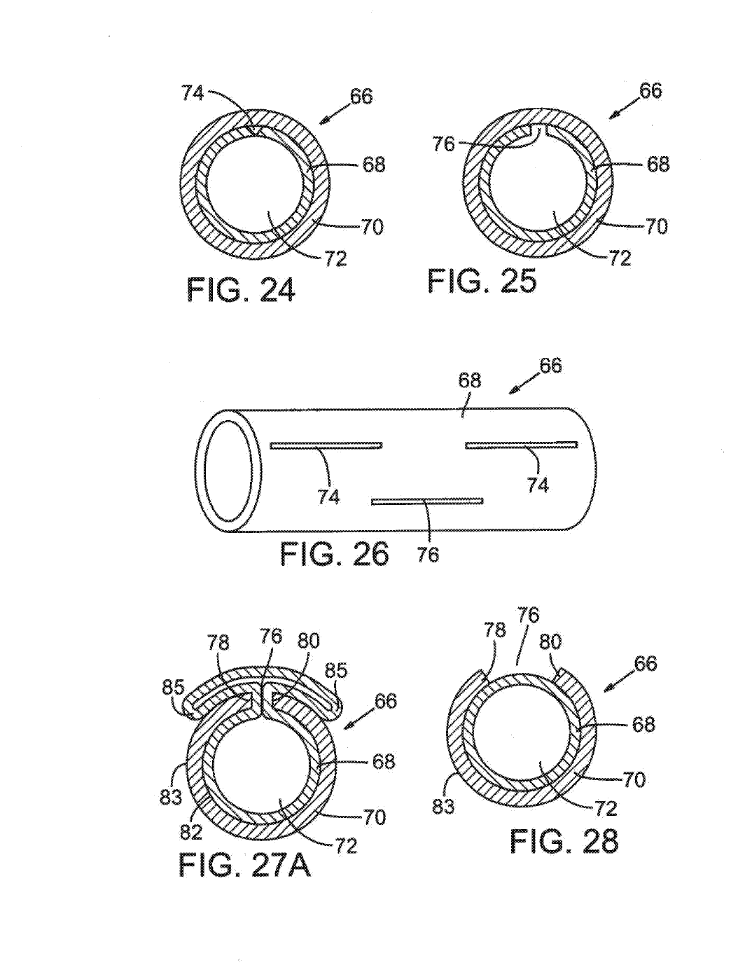

Alternative aspects of a sheath for introducing a prosthetic device are also described. For example, FIGS. 24-26 illustrate a section view and a perspective view, respectively, of a sheath 66 for introducing a prosthetic device into a body. The sheath 66 comprises an inner liner, such as inner polymeric layer 68, an outer layer, such as polymeric tubular layer 70, and a hemostasis valve (not shown). The inner polymeric layer 68 and the outer polymeric tubular layer 70 at least partially enclose a lumen 72, through which a delivery apparatus and prosthetic device can pass from outside the patient's body into the patient's vessel. Either or both of the inner polymeric layer 68 and the outer polymeric layer 70 can be provided with at least one longitudinal notch and/or cut to facilitate radial expansion of the sheath.

For example, FIG. 24 illustrates a longitudinal notch 74 in the inner polymeric layer 68 that can facilitate radial expansion of the sheath 66. The longitudinal notch 74 can separate or split open completely upon application of a radial force due to insertion of a delivery apparatus or prosthetic device. Similarly, FIG. 25 illustrates a longitudinal cut 76 in the inner polymeric layer 68 that can also facilitate radial expansion of the sheath 66. The outer polymeric layer 70 can, additionally or alternatively, comprise one or more longitudinal cuts 76 or notches 74. Such cuts and/or notches, whether in the inner polymeric layer 68 or the outer polymeric layer 70, can extend substantially through the entire thickness of the layer or can extend only partially through the thickness of the layer. The cuts and/or notches can be positioned at or near the inner or outer surface, or both surfaces, of the inner and/or outer polymeric layers 68, 70.

FIG. 26 illustrates a perspective view of one aspect of an inner polymeric layer 68 with longitudinal notches 74 and a longitudinal cut 76. More or fewer notches 74 and/or cuts 76 can be provided. For clarity, the outer polymeric layer 70 is not shown in FIG. 26. As shown in FIG. 26, longitudinal notches 74 and/or cuts 76 can extend only along a portion of the length of sheath 66. In alternative aspects, one or more notches 74 and/or cuts 76 can extend substantially along the entire length of the sheath 66. Additionally, notches 74 and/or cuts 76 can be positioned randomly or patterned.

One particular aspect of a sheath 66 comprises a sheath having a notch or cut in the outer polymeric layer 70 or the inner polymeric layer 68 that extends longitudinally along approximately 75% of the length of the sheath 66. If such a notch or cut extends only partially through the associated layer, it can have a relatively low tear force, such as a tear force of about 0.5 lbs., so that the notch splits open relatively easily during use.

The inner polymeric layer 68 and the outer polymeric layer 70 can optionally be adhered together or otherwise physically associated with one another. The amount of adhesion between the inner polymeric layer 68 and the outer polymeric layer 70 can be variable over the surfaces of the layers. For example, little to no adhesion can be present at areas around or near any notches and/or cuts present in the layers so as not to hinder the radial expansion of the sheath 66. Adhesion between the layers can be created by, for example, thermal bonding and/or coatings. In some aspects, a sheath 66 can be formed from an extruded tube, which can serve as the inner polymeric layer 68. The inner polymeric layer 68 can be surface-treated, such as by plasma etching, chemical etching, or other suitable methods of surface treatment. By treating the surface of the inner polymeric layer 68, the outer surface of the inner polymeric layer 68 can have areas with altered surface angles that can provide better adhesion between the inner polymeric layer 68 and the outer polymeric layer 70. The treated inner polymeric layer can be dip coated in, for example, a polyurethane solution to form the outer polymeric layer 70. In some configurations, the polyurethane may not adhere well to untreated surface areas of the inner polymeric layer 68. Thus, by surface treating only surface areas of the inner polymeric layer 68 that are spaced away from the areas of expansion (e.g., the portion of the inner polymeric layer 68 near notches 74 and/or cuts 76), the outer polymeric layer 70 can be adhered to some areas of the inner polymeric layer 68, while other areas of the inner polymeric layer 68 remain free to slide relative to the outer polymeric layer 70, thus allowing for expansion of the diameter of the sheath 66. Thus, areas around or near any notches 74 and/or cuts 76 can experience little to no adhesion between the layers, while other areas of the inner and outer polymeric layers 68, 70 can be adhesively secured or otherwise physically associated with each other.

As with previously disclosed aspects, the aspects illustrated in FIGS. 24-26 can be applied to sheaths having a wide variety of inner and outer diameters. Applications can utilize a sheath of the present disclosure with an inner diameter of the inner polymeric layer 68 that is expandable to an expanded diameter of from about 3 Fr to about 26 Fr. The expanded diameter can vary slightly along the length of the sheath 66. For example, the expanded outer diameter at the proximal end of the sheath 66 can range from about 3 Fr to about 28 Fr, while the expanded outer diameter at the distal end of the sheath 66 can range from about 3 Fr to about 25 Fr. In some aspects, a sheath 66 can expand to an expanded outer diameter that is from about 10% greater than the original unexpanded outer diameter to about 100% greater than the original unexpanded outer diameter.

In some aspects, the outer diameter of the sheath 66 gradually decreases from the proximal end of the sheath 66 to the distal end of the sheath 66. For example, in one aspect, the outer diameter can gradually decrease from about 26 Fr at the proximal end to about 18 Fr at the distal end. The diameter of the sheath 66 can transition gradually across substantially the entire length of the sheath 66. In other aspects, the transition or reduction of the diameter of the sheath 66 can occur only along a portion of the length of the sheath 66. For example, the transition can occur along a length from the proximal end to the distal end, where the length can range from about 0.5 inches to about the entire length of sheath 66.

Suitable materials for the inner polymeric layer 68 can have a high elastic strength and include materials discussed in connection with other aspects, especially Teflon (PTFE), polyethylene (e.g., high density polyethylene), fluoropolymers, or combinations thereof. In some aspects, the inner polymeric layer 68 preferably has a low coefficient of friction, such as a coefficient of friction from about 0.01 to about 0.5. Some exemplary aspects of a sheath 66 comprise an inner polymeric layer 68 having a coefficient of friction of about 0.1 or less.

Likewise, suitable materials for the outer polymeric layer 70 include materials discussed in connection with other aspects and other thermoplastic elastomers and/or highly elastic materials.

The Shore hardness of the outer polymeric layer 70 can be varied for different applications and aspects. Some aspects comprise an outer polymeric layer with a Shore hardness of from about 25A to about 80A, or from about 20D to about 40D. One particular aspect comprises a readily available polyurethane with a Shore hardness of 72A. Another particular aspect comprises a polyethylene inner polymeric layer dipped in polyurethane or silicone to create the outer polymeric layer.

The sheath 66 can also include a radiopaque filler or marker, as described above. In some aspects, a distinct radiopaque marker or band can be applied to some portion of the sheath 66. For example, a radiopaque marker can be coupled to the inner polymeric layer 68, the outer polymeric layer 70, and/or can be positioned in between the inner and outer polymeric layers 68, 70.

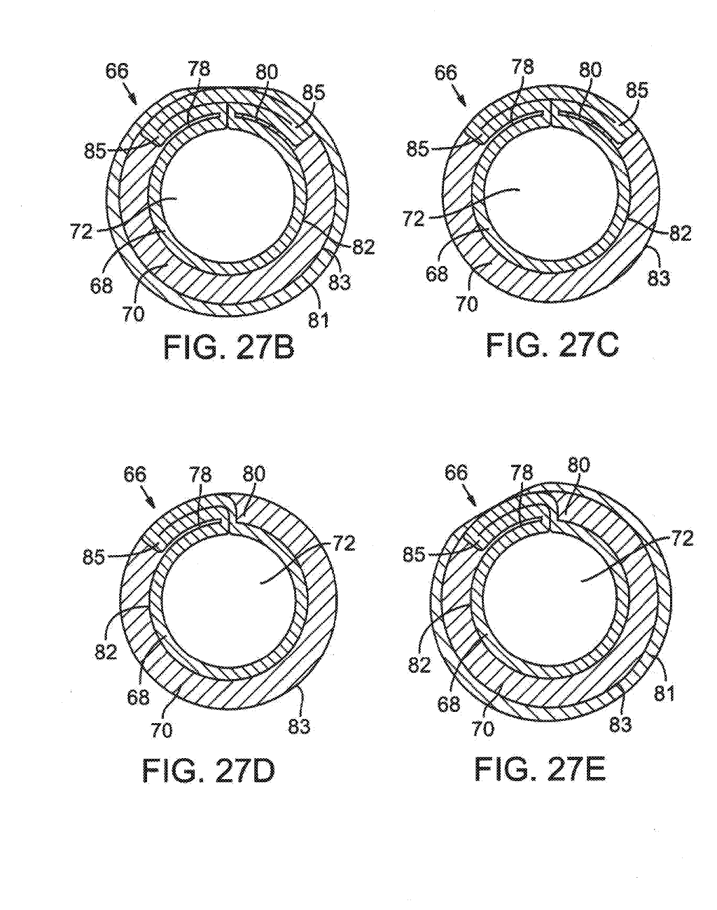

FIGS. 27A-27E and 28 illustrate section views of various aspects of unexpanded (FIGS. 27A-27E) and expanded (FIG. 28) sheaths 66 according to the present disclosure. The sheath 66 includes a split outer polymeric tubular layer 70 having a longitudinal cut 76 through the thickness of the outer polymeric tubular layer 70 such that the outer polymeric tubular layer 70 comprises a first portion 78 and a second portion 80 separable from one another along the cut 76. An expandable inner polymeric layer 68 is associated with an inner surface 82 of the outer polymeric tubular layer 70, and, in the unexpanded configuration shown in FIG. 27A, a portion of the inner polymeric layer 68 extends through a gap created by the cut 76 and can be compressed between the first and second portions 78, 80 of the outer polymeric tubular layer 70. Upon expansion of the sheath 66, as shown in FIG. 28, first and second portions 78, 80 of the outer polymeric tubular layer 70 have separated from one another, and the inner polymeric layer 68 is expanded to a substantially cylindrical tube. In some aspects, two or more longitudinal cuts 76 may be provided through the thickness of the outer polymeric tubular layer 70. In such aspects, a portion of the inner polymeric layer 68 may extend through each of the longitudinal cuts 76 provided in the outer polymeric tubular layer 70.

Preferably, the inner polymeric layer 68 comprises one or more materials that are elastic and amenable to folding and/or pleating. For example, FIG. 27A illustrates an inner polymeric layer 68 with folded regions 85. As seen in FIGS. 27A-27E, the sheath 66 can be provided with one or more folded regions 85. Such folded regions 85 can be provided along a radial direction and substantially conform to the circumference of the outer polymeric tubular layer 70. At least a portion of the folded regions 85 can be positioned adjacent the outer surface 83 of the outer polymeric tubular layer 70.

Additionally, as shown in FIGS. 27B and 27E, at least a portion of the folded region or regions 85 can be overlapped by an outer covering, such as outer polymeric covering 81. The outer polymeric covering 81 can be adjacent at least a portion of the outer surface 83 of the outer polymeric tubular layer 70. The outer polymeric covering 81 serves to at least partially contain the folded regions 85 of the inner polymeric layer 68 and can also prevent the folded regions 85 from separating from the outer polymeric tubular layer 70 when, for example, the sheath 66 undergoes bending. In some aspects, the outer polymeric covering 81 can be at least partially adhered to the outer surface 83 of the outer polymeric tubular layer 70. The outer polymeric covering 81 can also increase the stiffness and/or durability of the sheath 66.

Additionally, as shown in FIGS. 27B and 27E, the outer polymeric covering 81 may not entirely overlap the circumference of the sheath 66. For example, the outer polymeric covering 81 may be provided with first and second ends, where the ends do not contact one another. In these aspects, only a portion of the folded region 85 of the inner polymeric layer 68 is overlapped by the outer polymeric covering 81.

In aspects wherein a plurality of folded regions 85 are present, the regions can be equally displaced from each other around the circumference of the outer polymeric tubular layer 70. Alternatively, the folded regions can be off-center, different sizes, and/or randomly spaced apart from each other. While portions of the inner polymeric layer 68 and the outer tubular layer 70 can be adhered or otherwise coupled to one another, the folded regions 85 preferably are not adhered or coupled to the outer tubular layer 70. For example, adhesion between the inner polymeric layer 68 and the outer tubular layer 70 can be highest in areas of minimal expansion.

One particular aspect of the sheath illustrated in FIGS. 27A-28 comprises a polyethylene (e.g., high density polyethylene) outer polymeric tubular layer 70 and a PTFE inner polymeric layer 68. However, other materials are suitable for each layer, as described above. Generally, suitable materials for use with the outer polymeric tubular layer 70 include materials having a high stiffness or modulus of strength that can support expansion and contraction of the inner polymeric layer 68.

In some aspects, the outer polymeric tubular layer 70 comprises the same material or combination of materials along the entire length of the outer polymeric tubular layer 70. In alternative aspects, the material composition can change along the length of the outer polymeric tubular layer 70. For example, the outer polymeric tubular layer can be provided with one or more segments, where the composition changes from segment to segment. In one particular aspect, the Durometer rating of the composition changes along the length of the outer polymeric tubular layer 70 such that segments near the proximal end comprise a stiffer material or combination of materials, while segments near the distal end comprise a softer material or combination of materials. This can allow for a sheath 66 having a relatively stiff proximal end at the point of introducing a delivery apparatus while still having a relatively soft distal tip at the point of entry into the patient's vessel.

As with other disclosed aspects, the aspects of sheath 66 shown in FIGS. 27A-28 can be provided in a wide range of sizes and dimensions. For example, the sheath 66 can be provided with an unexpanded inner diameter of from about 3 Fr to about 26 Fr. In some aspects, the sheath 66 has an unexpanded inner diameter of from about 15 Fr to about 16 Fr. In some aspects, the unexpanded inner diameter of the sheath 66 can range from about 3 Fr to about 26 Fr at or near the distal end of sheath 66, while the unexpanded inner diameter of the sheath 66 can range from about 3 Fr to about 28 Fr at or near the proximal end of sheath 66. For example, in one aspect, the unexpended sheath 66 can transition from an unexpanded inner diameter of about 16 Fr at or near the distal end of the sheath 66 to an unexpanded inner diameter of about 26 Fr at or near the proximal end of the sheath 66.

The sheath 66 can be provided with an unexpanded outer diameter of from about 3 Fr to about 30 Fr, and, in some aspects, has an unexpanded outer diameter of from about 18 Fr to about 19 Fr. In some aspects, the unexpanded outer diameter of the sheath 66 can range from about 3 Fr to about 28 Fr at or near the distal end of sheath 66, while the unexpanded outer diameter of the sheath 66 can range from about 3 Fr to about 30 Fr at or near the proximal end of sheath 66. For example, in one unexpanded aspect, the sheath 66 can transition from an unexpanded outer diameter of about 18 Fr at or near the distal end of the sheath 66 to an unexpanded outer diameter of about 28 Fr at or near the proximal end of the sheath 66.

The thickness of the inner polymeric layer 68 can vary, but in some preferred aspects is from about 0.002 inches to about 0.015 inches. In some aspects, expansion of the sheath 66 can result in expansion of the unexpanded outer diameter of from about 10% or less to about 430% or more.

As with other illustrated and described aspects, the aspects shown in FIGS. 27A-28 can be provided with a radiopaque filler and/or a radiopaque tip marker, as described above. The sheath 66 can be provided with a radiopaque tip marker provided at or near the distal tip of the sheath 66. Such a radiopaque tip marker can comprise materials such as those suitable for the radiopaque filler, platinum, iridium, platinum/iridium alloys, stainless steel, other biocompatible metals, or combinations thereof.

FIGS. 50 and 51 show cross-sectional views of an expandable sheath 166 having an inner tubular layer 168 with a longitudinal slit 169. In some aspects, the longitudinal slit 169 extends the entire length of the inner tubular layer 168. An outer tubular layer 170 envelops the inner tubular layer 168 and includes a longitudinally extending, folded flap 171. In some aspects, the folded flap 171 extends the entire length of the outer tubular layer 170. The folded flap 171 overlies a portion of the outer surface 183 of the outer tubular layer 170 when the sheath is in an unexpanded state (FIG. 50). When a prosthetic device is moved through an inner lumen 172 of the sheath 166, it applies an outwardly directed radial force on the inner tubular layer 168 that widens the longitudinal slit 169 and unfolds the folded flap 171. FIG. 51 shows the sheath 166 in the unexpanded state, with the longitudinal slit 169 widened and the outer tubular layer 170 unfolded.

The folded flap 171 of the outer tubular layer 170 has a base 173. The base 173 can be positioned radially outwardly from the longitudinal slit 169. In some aspects, the base 173 is centered over the longitudinal slit 169. The folded flap 171 further includes a longitudinally extending overlying portion 175 extending from the base 173 to a longitudinally extending crease 179 at the edge of the flap 171. The longitudinally extending overlying portion 175 overlies a longitudinally extending underlying portion 177 and is separated from the underlying portion 177 by the crease 179. Underlying portion 177 contacts the outer surface 183 of the outer tubular layer 170 when the sheath is in an unexpanded state, as shown in FIG. 50.

Some aspects of sheath 166 can include multiple longitudinally extending folded flaps that overlie portions of the outer surface 183 of the outer tubular layer 170, positioned at various locations around the circumference of sheath 166. For example, 2, 3, 4, 5, 6, 7, 8, 9, or 10 longitudinally extending folded flaps can be positioned around the circumference. In some aspects, these multiple folded flaps are equally spaced around the circumference of the sheath 166.

The folded flap 171 extends circumferentially around a portion of the sheath 166. In some aspects, the longitudinally extending flap 171 extends around about 20% to about 40% of the outer circumference of the outer tubular layer 170 when the sheath 166 is unexpanded (including about 20%, about 21%, about 22%, about 23%, about 24%, about 25%, about 26%, about 2272%, about 28%, about 29%, about 30%, about 31%, about 32%, about 33%, about 34%, about 35%, about 36%, about 37%, about 38%, about 39%, and about 40% of the outer circumference of the outer tubular layer 170). In one example, for a sheath having a 14F (4.7 millimeters) unexpanded outer diameter, the folded flap extends around 85 to 120 degrees, or about 23%-35%, of the outer circumference (resulting in an inner lumen having an expanded diameter of about 7.6 millimeters to about 8.4 millimeters, which can be used with a valve having a 6.4 millimeter crimped outer diameter and a 26 millimeter expanded outer diameter).

In FIG. 51, the wall thickness of flap 171 is labeled t, and the wall thickness of other parts of the outer tubular layer 170 are labeled T. In some aspects, a portion of the flap 171 (such as, for example, the underlying portion 177 or the overlying portion 175) can have a wall thickness t that is thinner than a wall thickness (T) of other portions of the outer tubular layer. This variation in wall thickness promotes even column strength around the circumference of the sheath 166, which reduces kinking and minimizing the total outer diameter of the sheath. The wall thickness variation can also facilitate the folding process. In some aspects, the entire flap 171 has a wall thickness t that is thinner than a wall thickness T of the remainder of the outer tubular layer. In one example, the wall thickness of t can be from about 0.003 inches to about 0.007 inches, while the wall thickness of T can be from about 0.008 inches to about 0.012 inches. In other aspects, such as the one shown in FIG. 51, the wall thickness t of flap 171 is about equal to the wall thickness T of the remainder of the outer tubular layer 170.

The outer tubular layer 170 is formed of a material having a low coefficient of friction, a high tensile modulus, and a high ultimate tensile strength in order to improve the push force transmission through the length of sheath 166 while reducing kinking. Good push force transmission means that the force applied by the practitioner to advance the sheath is predictable, responsive, and consistent along the length of the sheath. However, an excessively high tensile modulus may limit the ability of the longitudinally extending flap 171 to open, which could hamper push force transmission. A desirable range for the tensile modulus of the outer tubular layer 170 is from about 300 MPa to about 2,000 MPa (including about 300 MPa, about 400 MPa, about 500 MPa, about 600 MPa, about 700 MPa, about 800 MPa, about 900 MPa, about 1000 MPa, about 1100 MPa, about 1200 MPa, about 1300 MPa, about 1400 MPa, about 1500 MPa, about 1600 MPa, about 1700 MPa, about 1800 MPa, about 1900 MPa, and about 2000 MPa). In some aspects, the tensile modulus may preferably be at least 700 MPa. High axial and radial stiffnesses enable the sheath to be easily inserted and to resist collapse within the body.

The ultimate tensile strength of the outer tubular layer 170 can be at least 50 MPa. A high ultimate tensile strength helps to prevent the material from tearing while the prosthetic device is advancing through the sheath.

Exemplary materials for formation of the outer tubular layer 170 of the aspects shown in FIGS. 50 and 51 include high density polyethylene (HDPE), polyamide, co-polyamide, polyether block amide (PEBAX®), or a blend of polyamide. Materials having shape memory properties are advantageous because the outer tubular layer 170 can be given a bias toward the folded state (for example, by heat setting). This facilitates refolding of the outer tubular layer 170 after passage of a prosthetic device. PEBAX® is an exemplary shape memory material that may be heat set toward the folded state.

In some aspects, the outer surface 183 of the outer tubular layer 170 can include a hydrophilic coating. In some aspects, a bond can be created between the underlying portion 177 of the folded flap 171 and the outer surface 183 of the outer tubular layer 170. The bond can be a thermal bond (with portions of the contacting layers melted together), or it can be a separate layer of adhesive.

As discussed above, the inner tubular layer 168 of the aspects shown in FIGS. 50 and 51 include a longitudinal slit 169. The inner tubular layer 168 includes a first longitudinally extending end 178 and a second longitudinally extending end 180, the first and second longitudinally extending ends 178, 180 defining the longitudinal slit 171.

The inner tubular layer 168 forms a low friction barrier between a passing prosthetic device and the higher friction outer tubular layer 170. The inner tubular layer 168 extends around at least 80% (or at least 85%, or at least 90%, or at least 95%) of the circumference of the inner lumen 172 when the sheath 166 is in an unexpanded state. This high degree of coverage limits contact between the passing prosthetic device and the higher friction outer tubular layer 170. In some aspects, the coefficient of friction per ASTM D1894 of the inner tubular layer 168 (static or dynamic) is 0.30 or less. In other aspects, the coefficient of friction is 0.25 or less.

In some aspects, the low-friction inner tubular layer 168 includes, or is formed of, a material having a tensile modulus of at least about 300 MPa (and up to about 1400 MPa) to provide good push force transmission while providing kink resistance. This material could be, for example, high density polyethylene or a fluoropolymer. Exemplary fluoropolymers include polytetrafluoroethylene, ethylene fluorinated ethylene propylene, or perfluoro alkoxy.

A tie layer 174 can be positioned between the two layers, thereby adhering the inner tubular layer 168 to the outer tubular layer 170. In some aspects, the tie layer 174 can be formed of polyurethane or functionalized polyolefin. In some aspects, the contacting surface of the inner tubular layer 168 can be etched to improve bonding to the tie layer. For example, an inner tubular layer 168 that includes, or is formed of, a fluoropolymer might be etched on its outer surface to improve thermal bonding to the tie layer 174.

Some aspects of the sheath 166 shown in FIGS. 50 and 51 can also include an outer jacket 181, as shown in FIG. 52. The outer jacket 181 is elastomeric (having a relatively low tensile modulus compared to the inner and outer tubular layers 168, 170). As mentioned above, in some aspects, the outer tubular layer 170 can be included or can be formed of a shape memory material (for example, a heat-set polymer such as PEBAX®) that facilitates refolding of the outer tubular layer 170 after expansion. In some aspects, the sheath 166 can include an elastomeric outer jacket 181 that extends over and envelops the outer tubular layer 170. The outer jacket 181 and the shape memory feature can be used alone or in conjunction with each other to facilitate refolding (i.e., the elastomeric outer jacket 181 can extend over an outer tubular layer 170 that comprises a shape memory material). The refolding of the outer tubular layer 170 can preferably return the sheath 166 back to its original outer diameter or to a value close to the original outer diameter (for example, to within about 10%, about 20%, about 30%, about 40%, or about 50% of the original outer diameter). The elastomeric outer jacket 181 can include or can be formed completely of materials such as low durometer polyurethane (for example, less than Shore 85A), styrene elastomer (for example, less than Shore 85A), latex, or low durometer PEBAX® (for example, less than Shore 35D).

Methods of using the sheath of FIGS. 50-52 include first inserting an expandable sheath 166 into the vasculature of a subject and advancing the prosthetic device through an inner lumen 172 of the expandable sheath 166. The prosthetic device applies an outwardly directed radial force to the inner tubular layer 168 of the expandable sheath 166. In some aspects, the outwardly directed radial force is transmitted through the inner tubular layer 168, the tie layer 174, and the outer tubular layer 170. The outwardly directed radial force widens a longitudinal slit 169 in the inner tubular layer 168. In certain aspects, the widening of the longitudinal slit 169 travels the full length of the expandable sheath.

The outwardly directed radial force further unfolds the longitudinally extending flap 171 of the outer tubular layer 170 to expand the expandable sheath. The unfolding of the flap 171 can include sliding a longitudinally extending overlying portion 175 circumferentially against a longitudinally extending underlying portion 177 of flap 171. The underlying portion 177 can slide circumferentially against an outer surface 183 of the outer tubular layer 170. In some aspects, the unfolding of the longitudinally extending flap 171 occurs at a position radially outward from the longitudinal slit 169 of the inner tubular layer 168. In still further aspects, the unfolding of the flap 171 can extend the full length of the expandable sheath 166.

The longitudinal slit 169 of the inner tubular layer 168 narrows once the outwardly directed radial force has ceased (i.e., once the prosthetic device has passed by). The slit 169 may narrow back to its original width or to a value close to the original width (for example, to a value within about 10% of the original width). The narrowing can occur along the entire length of the sheath 166. The prosthetic device is then delivered to the procedure site.

The longitudinally extending flap 171 at least partially refolds once the prosthetic device ceases to apply the outwardly directed radial force (i.e., once it has passed by). In some aspects, the longitudinally extending flap 171 refolds itself due to a shape memory bias toward the folded state. In some aspects, an inwardly directed radial force is applied to the outer surface 183 of the outer tubular layer 170 to refold the longitudinally extending flap 171 (for example, by elastomeric outer jacket 181).

An example method of making the sheath is as follows. These steps are not meant to be limiting. The steps given can be reordered as needed. Other steps can be added, or in other examples, some steps may not be necessary. Sizes are approximate. 1) Start with a PTFE inner liner of about 0.200 inch inner diameter (ID), wall thickness about 0.004 inches, 2) load PTFE inner liner on to a tapering mandrel from about 0.200 inches to about 0.187 inches, 3) stretch on to an 0.187 inch outer diameter (OD) mandrel section under heat, 4) flare proximal end of 0.200 inch ID PTFE to 0.340 inches ID under heat, 5) by expanding with air pressure, load tie layer such as Tecoflex 80A having about 0.200 inch ID and 0.004 inch wall thickness over the PTFE inner liner along the body section (optionally the tie layer can be applied after expanding the inner layer with air pressure), 6) adhere tie layer to inner liner, for example, by covering it with FEP (fluorinated ethylene propylene) heat shrink tubing and applying heat, 7) remove FEP thermal shrink tubing if it was used, 8) create a longitudinal slit along the body section of the assembly, 9) load the subassembly having a slit on to a 0.187 inch OD mandrel, 10) load outer layer over the body, 11) fold outer layer, 12) heat set the fold, for example, by inserting the subassembly with a fold inside a heat shrink tubing and placing the assembly into the oven, 13) remove the shrink tubing if it was used, 14) remove sheath from the mandrel. In some aspects, an outer elastomeric jacket is added over the heat set outer layer prior to removal from the mandrel.

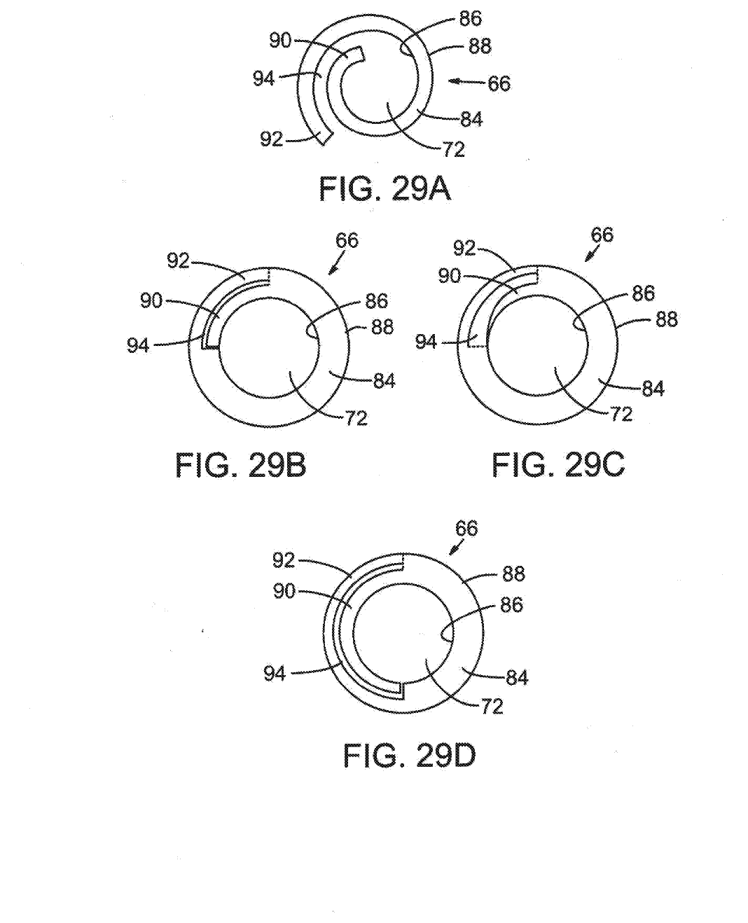

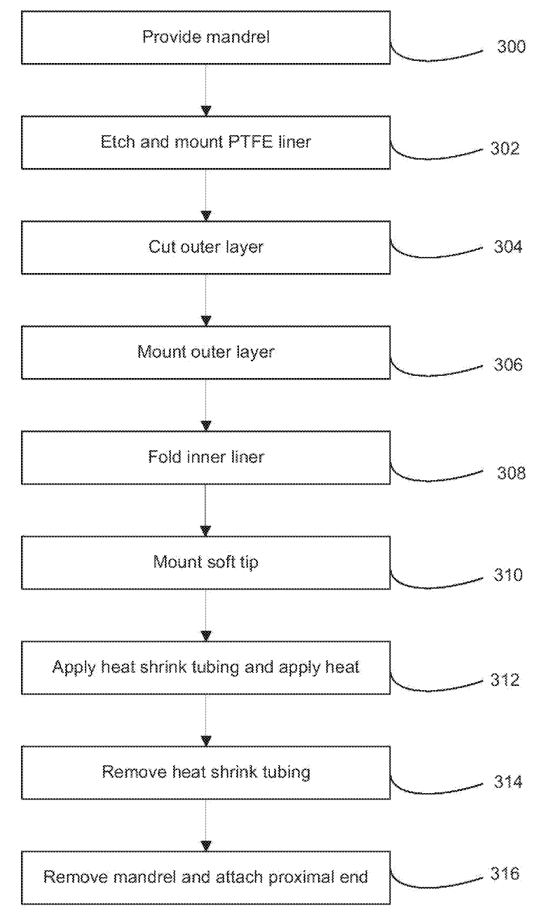

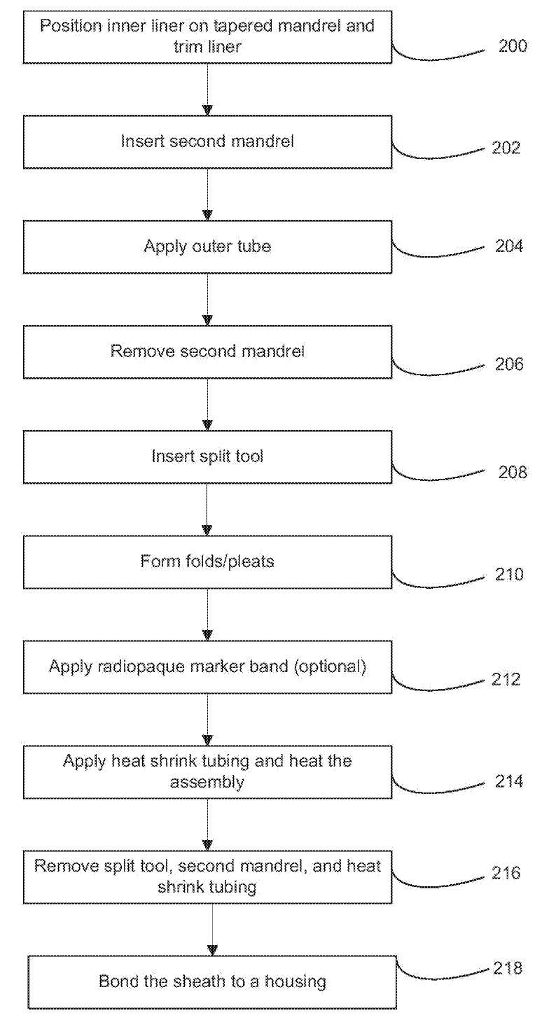

FIGS. 29A-29D show section views of other possible configurations of a sheath 66 for introducing a prosthetic device into a patient's vasculature. The sheath 66 comprises a polymeric tubular layer 84 having an inner surface 86 and an outer surface 88. The thickness of the polymeric tubular layer 84 extends from the inner surface 86 to the outer surface 88. As shown in FIGS. 29B-29D, the polymeric tubular layer 84 can be formed with at least a first angular portion 90 of reduced thickness adjacent the inner surface 86 and a second angular portion 92 of reduced thickness adjacent the outer surface 88, with the second portion 92 at least partially overlapping the first portion 90. FIG. 29A illustrates a similar configuration, where a second portion 92 at least partially overlaps a first portion 90 in a partial coil configuration. In the aspect of FIG. 29A, the second portion 92 and the first portion 90 can have the same thickness.

In preferred aspects, the first and second portions 90, 92 are not adhered to one another. In some aspects, and as best seen in FIG. 29A, there can be a small gap 94 between the first and second portions 90, 92 that can give the sheath 66 the appearance of having two interior lumens 72, 94. FIGS. 29A-29D illustrate the sheath 66 in unexpanded configurations. Preferably, upon expansion of the sheath 66, the ends of the first and second portions 90, 92 abut or are in close proximity to each other to reduce or eliminate any gap between them.

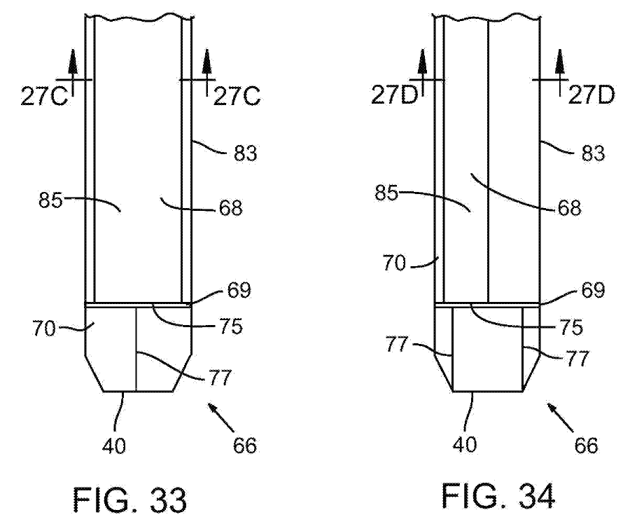

In some aspects, a sheath 66 can comprise a partial slit or score line along at least a portion of its length. For example, as shown in FIG. 33, a sheath 66 can comprise an outer polymeric tubular layer 70 over an inner polymeric layer 68. The inner polymeric layer can extend through a cut in the outer polymeric tubular layer 70 to form a folded region 85 on the outer surface of the outer polymeric tubular layer 70, such as also shown in FIG. 27C. The folded region 85 of the inner liner, in some aspects, terminates before the outer polymeric tubular layer 70 (i.e., the outer polymeric tubular layer 70 is longer than the inner liner). As shown in FIG. 33, in these aspects, the sheath 66 can comprise a partial slit or score line 77 that can extend from the termination (distal end) 75 of the folded region 85 to the distal end 40 of the sheath 66. In some aspects, score line 77 can facilitate expansion of the sheath 66.

Score line 77 can be substantially centrally located with respect to the folded region 85. In alternative aspects, score line 77 can be positioned in other locations relative to the folded region 85. Also, sheath 66 can comprise one or more score lines 77. For example, as shown in FIG. 34, one or more score lines 77 can be peripherally located with respect to the folded region 85. The one or more score lines 77 can be positioned anywhere around the circumference of the outer polymeric tubular layer 70. In aspects comprising a radiopaque marker 69 as seen in FIG. 33, a score line 77 can extend from, for example, the distal end of the radiopaque marker 69 substantially to the distal end 40 of the sheath 66.

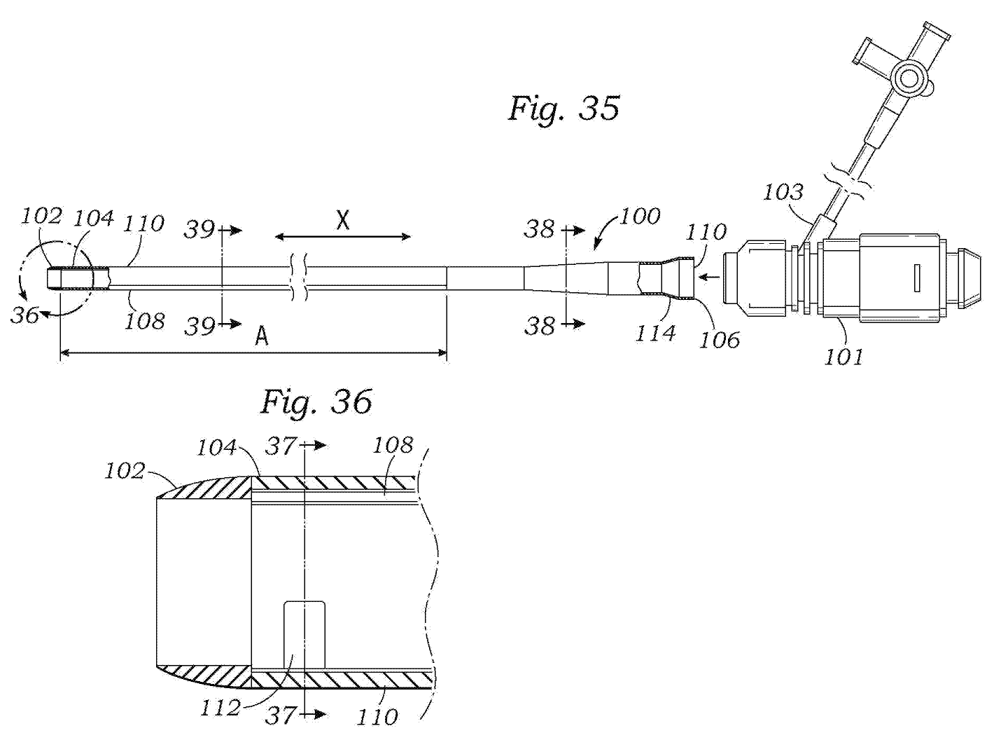

FIGS. 35 and 36 illustrate an expandable sheath 100 according to the present disclosure, which can be used with a delivery apparatus for delivering a prosthetic device, such as a tissue heart valve, into a patient. In general, the delivery apparatus can include a steerable guide catheter (also referred to as a flex catheter) a balloon catheter extending through the guide catheter, and a nose catheter extending through the balloon catheter (e.g., as depicted in FIG. 1). The guide catheter, the balloon catheter, and the nose catheter can be adapted to slide longitudinally relative to each other to facilitate delivery and positioning of the valve at an implantation site in a patient's body. However, it should be noted that the sheath 100 can be used with any type of elongated delivery apparatus used for implanting balloon-expandable prosthetic valves, self-expanding prosthetic valves, and other prosthetic devices. Generally, sheath 100 can be inserted into a vessel (e.g., the femoral or iliac arteries) by passing through the skin of the patient, such that a soft tip portion 102 at the distal end 104 of the sheath 100 is inserted into the vessel. The sheath 100 can also include a proximal flared end portion 114 to facilitate mating with an introducer housing 101 and catheters mentioned above (e.g., the proximal flared end portion 114 can provide a compression fit over the housing tip and/or the proximal flared end portion 114 can be secured to the housing 101 via a nut or other fastening device or by bonding the proximal end of the sheath to the housing). The introducer housing 101 can house one or more valves that form a seal around the outer surface of the delivery apparatus once inserted through the housing, as known in the art. The delivery apparatus can be inserted into and through the sheath 100, allowing the prosthetic device to be advanced through the patient's vasculature and implanted within the patient.

Sheath 100 can include a plurality of layers. For example, sheath 100 can include an inner liner 108 and an outer layer 110 disposed around the inner liner 108. The inner liner 108 can define a lumen through which a delivery apparatus can travel into a patient’s vessel in order to deliver, remove, repair, and/or replace a prosthetic device, moving in a direction along the longitudinal axis X. As the prosthetic device passes through the sheath 100, the sheath locally expands from a first, resting diameter to a second, expanded diameter to accommodate the prosthetic device. After the prosthetic device passes through a particular location of the sheath 100, each successive expanded portion or segment of the sheath 100 at least partially returns to the smaller, resting diameter. In this manner, the sheath 100 can be considered self-expanding in that it does not require the use of a balloon, dilator, and/or obturator to expand.

The inner and outer layers 108, 110 can comprise any suitable materials. Suitable materials for the inner liner 108 include polytetrafluoroethylene (PTFE), ethylene tetrafluoroethylene (ETFE), nylon, polyethylene, polyether block amide (e.g., PEBAX®), and/or combinations thereof. In some exemplary aspects, the inner liner 108 can comprise a lubricious, low-friction, or hydrophilic material, such as PTFE. Such a low coefficient of friction materials can facilitate passage of the prosthetic device through the lumen defined by the inner liner 108. In other aspects, the inner liner 108 can have a coefficient of friction of less than about 0.1. Some aspects of a sheath 100 can include a lubricious liner on the inner surface of the inner liner 108. Examples of suitable lubricious liners include materials that can further reduce the coefficient of friction of the inner liner 108, such as PTFE, polyethylene, polyvinylidene fluoride, and combinations thereof. Suitable materials for a lubricious liner also include other materials desirably having a coefficient of friction of about 0.1 or less.

Suitable materials for the outer layer 110 include nylon, polyethylene, PEBAX®, HDPE, polyurethanes (e.g., Tecoflex™), and other medical grade materials. In one aspect, the outer layer 110 can comprise high density polyethylene (HDPE) and Tecoflex™ (or other polyurethane material) extruded as a composite. In some aspects, the Tecoflex™ can act as an adhesive between the inner liner 108 and the outer layer 110 and may only be present along a portion of the inner surface of the outer layer 110. Other suitable materials for the inner and outer layers are also disclosed in U.S. Patent Application Publication No. 2010/0094392, which is incorporated herein by reference.

Additionally, some aspects of a sheath 100 can include an exterior hydrophilic coating on the outer surface of the outer layer 110. Such a hydrophilic coating can facilitate insertion of the sheath 100 into a patient’s vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath 100.

Best seen in FIG. 36, the soft tip portion 102 can comprise, in some aspects, low-density polyethylene (LDPE) and can be configured to minimize trauma or damage to the patient’s vessels as the sheath is navigated through the vasculature. For example, in some aspects, the soft tip portion 102 can be slightly tapered to facilitate passage through the vessels. The soft tip portion 102 can be secured to the distal end 104 of the sheath 100, such as by thermally bonding the soft tip portion 102 to the inner and outer layers of the sheath 100. Such a soft tip portion 102 can be provided with a lower hardness than the other portions of the sheath 100. In some aspects, the soft tip 102 can have a Shore hardness from about 25 D to about 40 D. The tip portion 102 is configured to be radially expandable to allow a prosthetic device to pass through the distal opening of the sheath 100. For example, the tip portion 102 can be formed with a weakened portion, such as an axially extending score line or perforated line that is configured to split and allow the tip portion to expand radially when the prosthetic device passes through the tip portion (such as shown in the aspects of FIGS. 33 and 34).

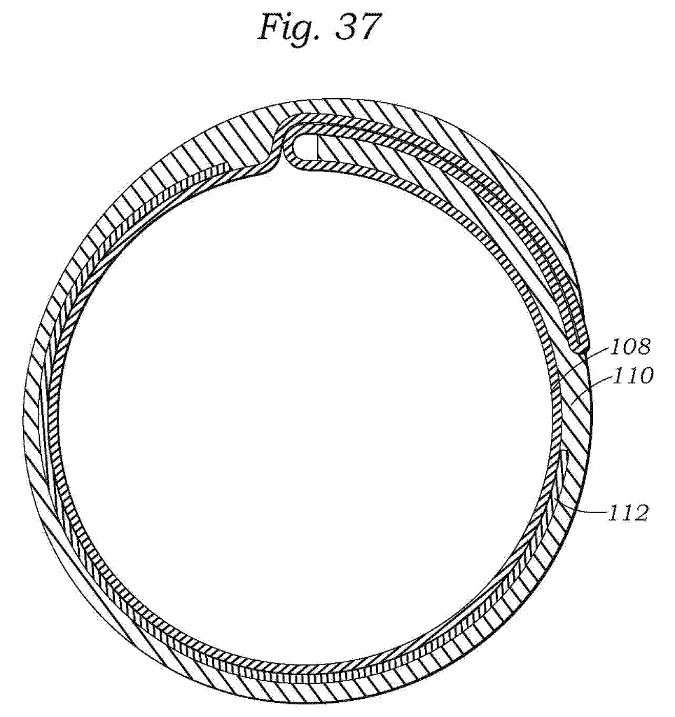

FIG. 37 shows a cross-section view of the sheath 100 taken near the distal end 104 of the sheath 100. As shown in FIGS. 36 and 37, the sheath 100 can include at least one radiopaque filler or marker, such as a discontinuous, or C-shaped, band 112 positioned near the distal end 104 of the sheath 100. The marker 112 can be associated with the inner and/or outer layers 108, 110 of the sheath 100. For example, as shown in FIG. 37, the marker 112 can be positioned between the inner liner 108 and the outer layer 110. In alternative aspects, the marker 112 can be associated with the outer surface of the outer layer 110. In some aspects, the marker 112 can be embedded or blended within the inner or outer layers 108, 110.

The C-shaped band 112 can serve as a radiopaque marker or filler to enable visibility of the sheath 100 under fluoroscopy during use within a patient. The C-shaped band 112 can comprise any suitable radiopaque material, such as barium sulfite, bismuth trioxide, titanium dioxide, bismuth subcarbonate, platinum, iridium, and combinations thereof. In one specific aspect, the C-shaped band can comprise about 90% platinum and about 10% iridium. In other aspects, the marker 112 needs not to be a C-shaped band. Other shapes, designs, and configurations are possible. For example, in some aspects, the marker 112 can extend around the entire circumference of the sheath 100. In other aspects, the marker 112 can comprise a plurality of small markers spaced around the sheath 100.

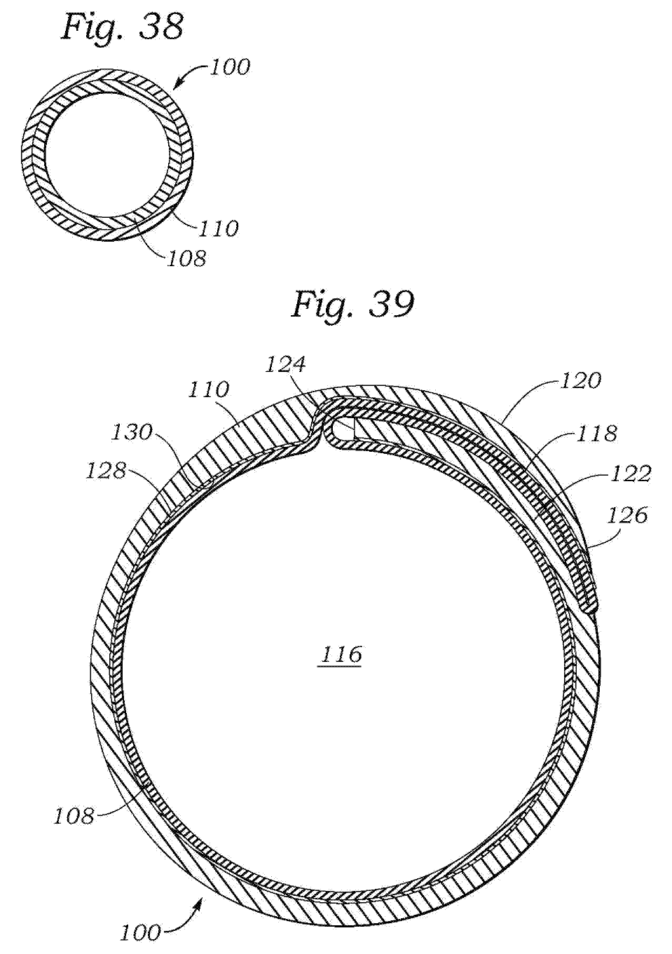

FIGS. 38 and 39 show additional cross sections taken at different points along the sheath 100. FIG. 38 shows a cross-section of a segment of the sheath near the proximal end 106 of the sheath 100, as indicated by line 38-38 in FIG. 35. The sheath 100 at this location can include inner liner 108 and outer layer 110. At this location, near the proximal end of the sheath, the layers 108, 110 can be substantially tubular, without any slits or folded portions in the layers. By contrast, the layers 108, 110 at different locations along the sheath 100 (e.g., at the point indicated by line 39-39 in FIG. 35) can have a different configuration.

As shown in FIG. 39, the inner liner 108 can be arranged to form a substantially cylindrical lumen 116 therethrough. Inner liner 108 can include one or more folded portions 118. In the aspect shown in FIG. 39, inner liner 108 is arranged to have one folded portion 118 that can be positioned on either side of the inner liner 108. The folded portion 118 includes a first fold (e.g., a longitudinally extending fold line) and a second fold and an overlapping portion extending circumferentially therebetween (when the sheath is in an unexpanded configuration). As illustrated in FIG. 39, the folded portion 118 comprises an overlap in a radial direction of at least two thicknesses of the inner liner 108. Inner liner 108 can be continuous in that there are no breaks, slits, or perforations in inner liner 108. Outer layer 110 can be arranged in an overlapping fashion such that an overlapping portion 120 overlaps at least a part of the folded portion 118 of the inner liner 108. As shown in FIG. 39, the overlapping portion 120 also overlaps an underlying portion 122 of the outer layer 110. The underlying portion 122 can be positioned to underlie both the overlapping portion 120 of the outer layer 110, as well as the folded portion 118 of the inner liner 108. Thus, the outer layer 110 can be discontinuous in that it includes a slit or a cut in order to form the overlapping and underlying portions 120, 122. In other words, a first edge 124 of the outer layer 110 is spaced apart from a second edge 126 of the outer layer 110 so as not to form a continuous layer.

As shown in FIG. 39, the sheath 100 can also include a thin layer of bonding or adhesive material 128, also referred to as a tie layer, positioned between the inner and outer layers 108, 110. In one aspect, the adhesive material 128 can comprise a polyurethane material such as Tecoflex™ or etched PTFE tubing. The adhesive material 128 can be positioned on an inner surface 130 of at least a portion of the outer layer 110 so as to provide adhesion between selected portions of the inner and outer layers 108, 110. For example, the outer layer 110 may only include an adhesive layer 128 around the portion of the inner surface 130 that faces the lumen-forming portion of the inner liner 108. In other words, the adhesive layer 128 can be positioned so that it does not contact the folded portion 118 of the inner liner 108 in some aspects. In other aspects, the adhesive layer 128 can be positioned in different configurations as desired for the particular application. For example, as shown in FIG. 39, the adhesive layer 128 can be positioned along the entire inner surface 130 of the outer layer 110. In an alternative aspect, the adhesive layer can be applied to the outer surface of the inner liner 108 instead of the inner surface of the outer layer. The adhesive layer 128 can be applied to all or selected portions on the inner liner 108; for example, the adhesive layer 128 can be formed only on the portion of the inner liner that faces the lumen-forming portion of the outer layer and not on the folded portion. The configuration of FIG. 39 allows for radial expansion of the sheath 100 as an outwardly directed radial force is applied from within (e.g., by passing a medical device such as a prosthetic heart valve through the lumen 116). As radial force is applied, the folded portion 118 can at least be partially separated, straighten, and/or unfold, and/or the overlapping portion 120 and the underlying portion 122 of the outer layer 110 can slide circumferentially with respect to one another, thereby allowing the diameter of lumen 116 to enlarge.

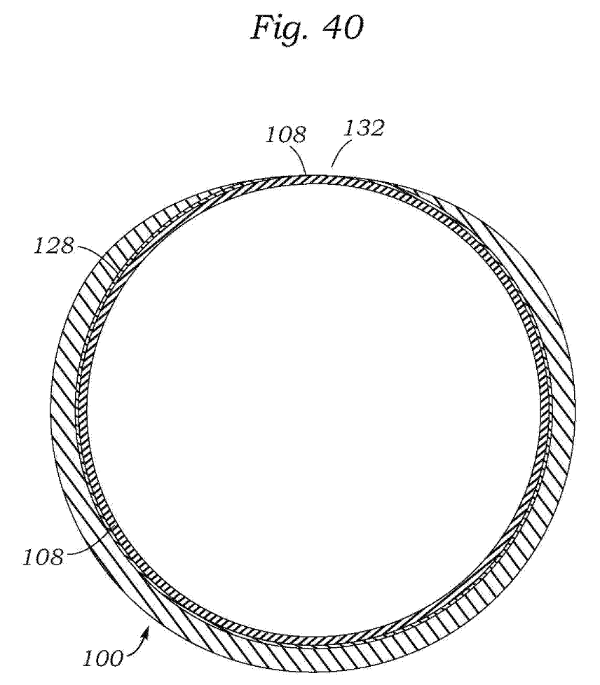

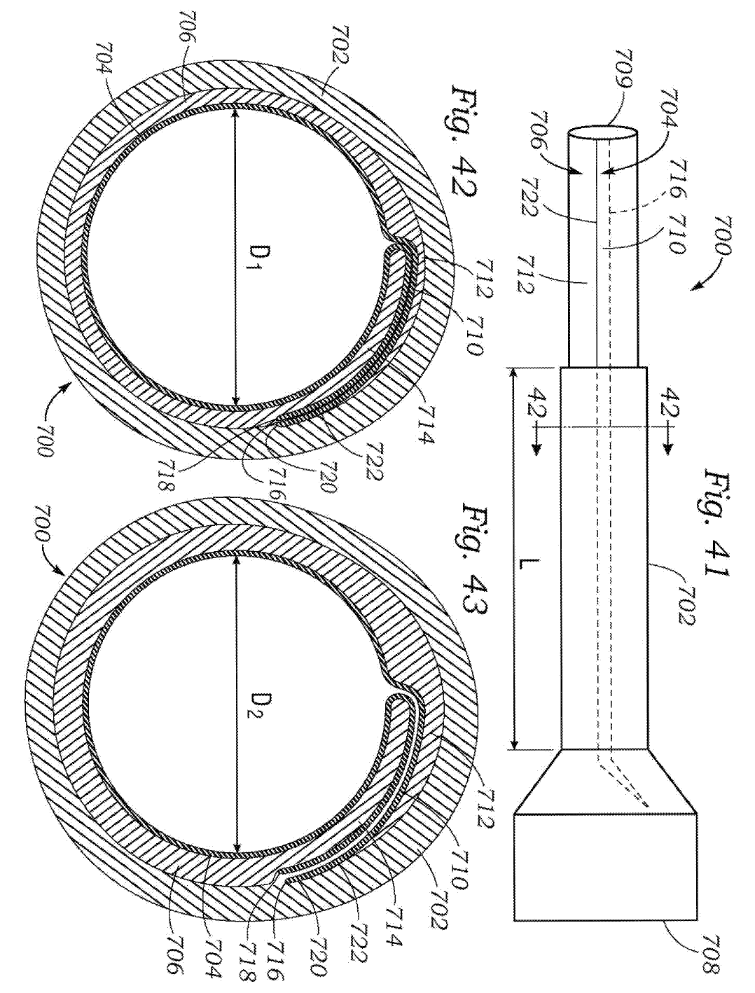

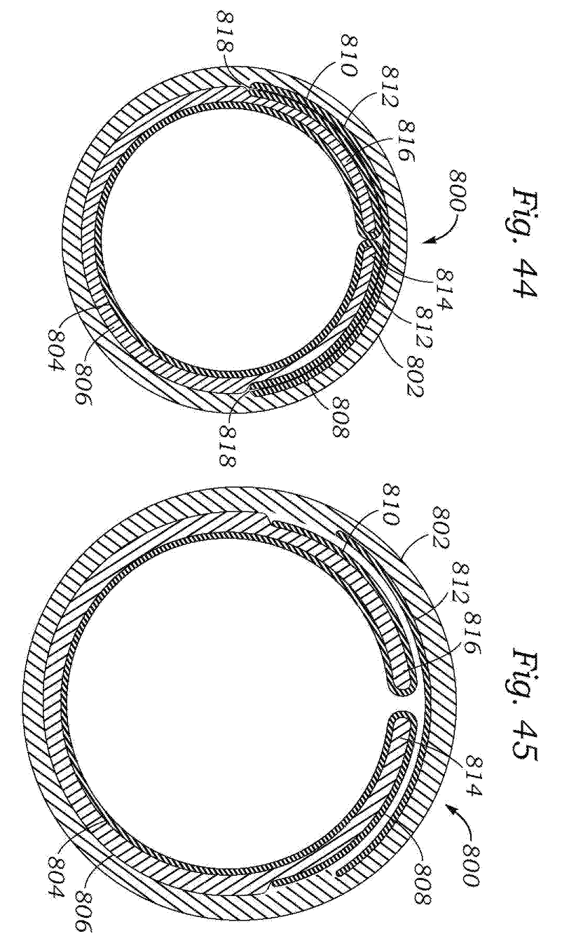

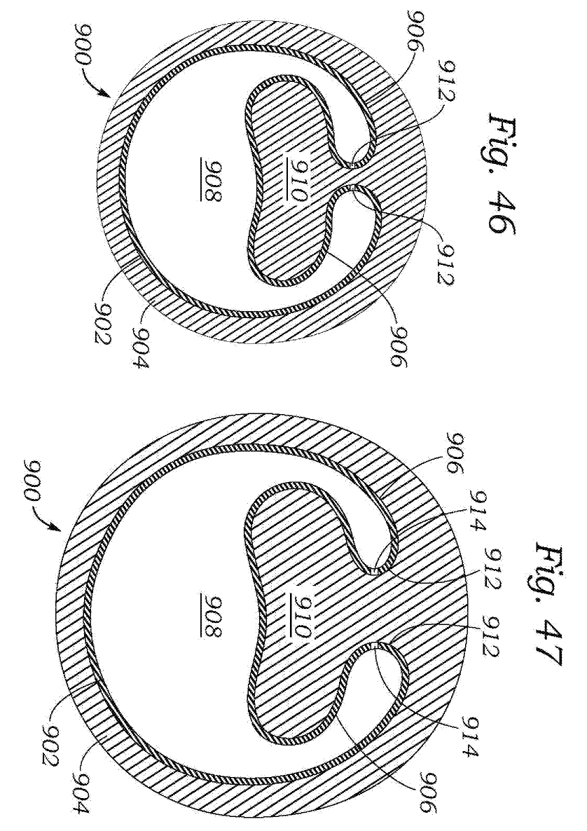

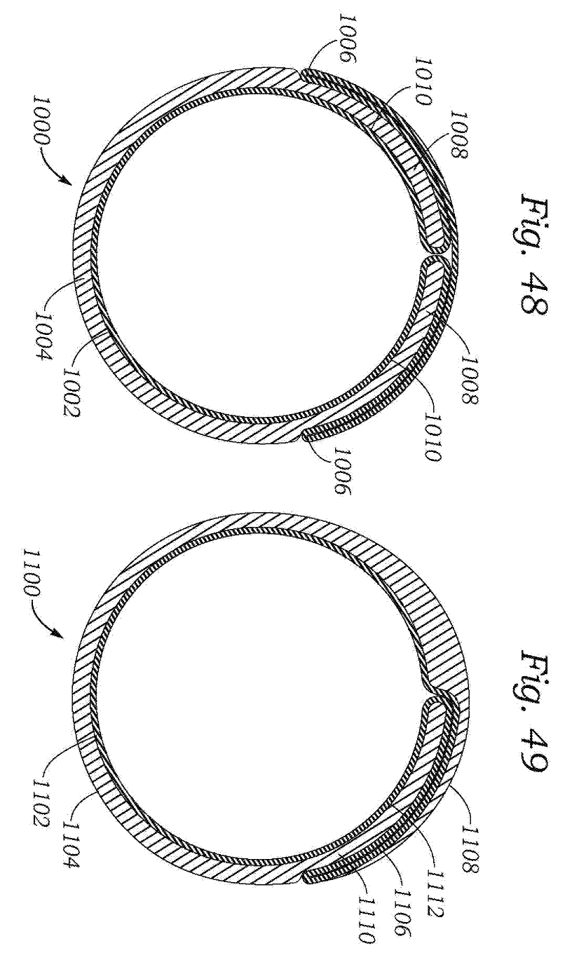

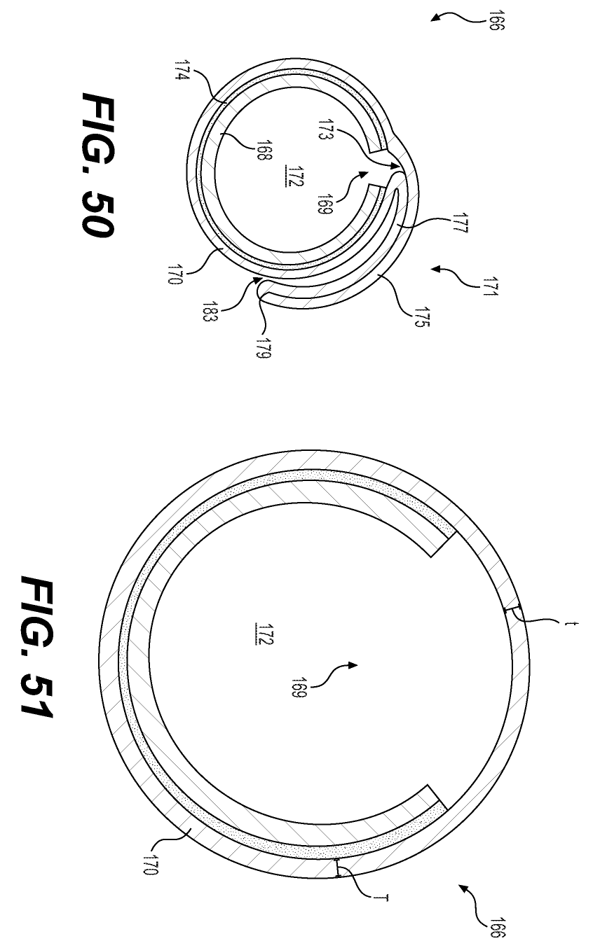

In this manner, the sheath 100 is configured to expand from a resting configuration (FIG. 39) to an expanded configuration shown in FIG. 40. In the expanded configuration, as shown in FIG. 40, an annular gap 132 can form between the longitudinal edges of the overlapping portion 120 and the underlying portion 122 of the outer layer 110. As the sheath 100 expands at a particular location (i.e., locally expands at the location of the passing prosthetic device), the overlapping portion 120 of the outer layer 110 can move circumferentially with respect to the underlying portion 122 as the folded portion 118 of the inner liner 108 unfolds. This movement can be facilitated by the use of a low-friction material for inner liner 108, such as PTFE. Further, the folded portion 118 can at least partially separate and/or unfold to accommodate a medical device having a diameter larger than that of lumen 116 in the resting configuration. As shown in FIG. 40, in some aspects, the folded portion of the inner liner 108 can completely unfold so that the inner liner 108 forms a cylindrical tube at the location of the expanded configuration.

The sheath 100 can be configured such that it locally expands at a particular location corresponding to the location of the medical device along the length of the lumen 116, and then locally contracts once the medical device has passed that particular location. Thus, a bulge may be visible, traveling longitudinally along the length of the sheath as a medical device is introduced through the sheath, representing continuous local expansion and contraction as the device travels the length of the sheath 100. In some aspects, each segment of the sheath 100 can locally contract after removal of any radial outward force such that it regains the original resting diameter of lumen 116. In some aspects, each segment of the sheath 100 can locally contract after removal of any radial outward force such that it at least partially returns to the original resting diameter of lumen 116.

The layers 108, 110 of sheath 100 can be configured, as shown in FIG. 39 along at least a portion of the length of the sheath 100. In some aspects, the layers 108, 110 can be configured as shown in FIG. 39 along the length A (FIG. 35) extending from a location adjacent the soft tip portion 102 to a location closer to the proximal end 106 of the sheath 100. In this matter, the sheath is expandable and contractable only along a portion of the length of the sheath corresponding to length A (which typically corresponds to the section of the sheath inserted into the narrowest section of the patient's vasculature).

The sheath 100 of FIG. 35 can include an outer jacket 140 extending over the outer layer 110. FIGS. 53 and 54 show additional cross sections taken at different points along the sheath 100. Similar to FIG. 38, FIG. 53 shows a cross-section of a segment of the sheath near the proximal end 106 of the sheath 100, as indicated by line 38-38 in FIG. 35. The sheath 100 at this location can include inner liner 108, outer layer 110, adhesive layer 128, and an outer jacket 140. At this location, near the proximal end of the sheath, the layers 108, 110, and outer jacket 140 can be substantially tubular, without any slits or folded portions in the layers. By contrast, the layers 108, 110 at different locations along the sheath 100 (e.g., at the point indicated by line 39-39 in FIG. 35) can have a different configuration, while the outer jacket 140 maintains a substantially tubular shape, without slits or folds.

As shown in FIG. 54, and described above with respect to FIG. 39, the inner liner 108 can be arranged to form a substantially cylindrical lumen 116 extending therethrough. The inner liner 108 can include one or more folded portions 118. The outer layer 110 can be arranged in an overlapping fashion such that an overlapping portion 120 overlaps at least a part of the folded portion 118 of the inner liner 108 as well as the underlying portion 122 (positioned to underlie the folded portion 118 of the inner liner 108) when the sheath is unexpanded. The sheath 100 is configured to locally expand from an unexpanded configuration in which the lumen 116 has a first diameter to an expanded configuration in which the lumen 116 has a second diameter larger than the first diameter. The sheath 100 expands in response to an outwardly directed radial force exerted by a medical device against the inner liner 108 as it passes through the lumen 116. During expansion, the first fold/folded edge moves closer to the second fold/folded edge to shorten the folded portion 118. As shown in FIG. 55, in some aspects, the folded portion 118 of the inner liner 108 can completely unfold so that the inner liner 108 forms a cylindrical tube at the location of the expanded configuration. When the sheath is expanded, a portion of the inner liner 108 extends through the opening/gap provided in the outer layer 110, where the opening is formed by the longitudinally extending edge of the overlapping portion 120 and a longitudinally extending edge of the underlying portion 122. As the prosthetic device passes, the sheath 100 then locally contracts at least partially back to the unexpanded configuration.

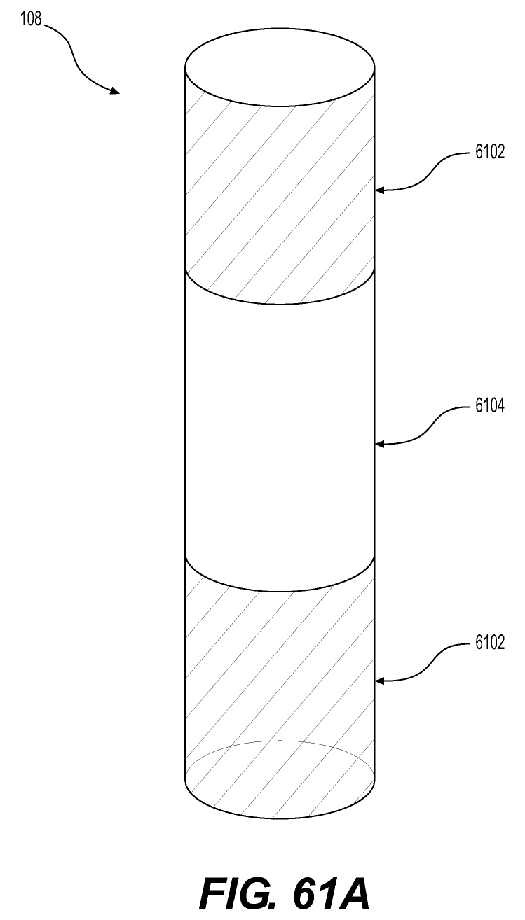

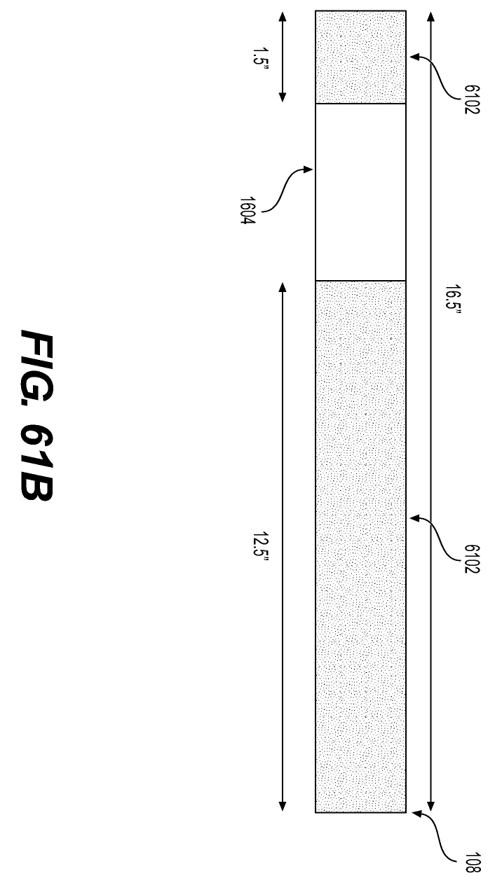

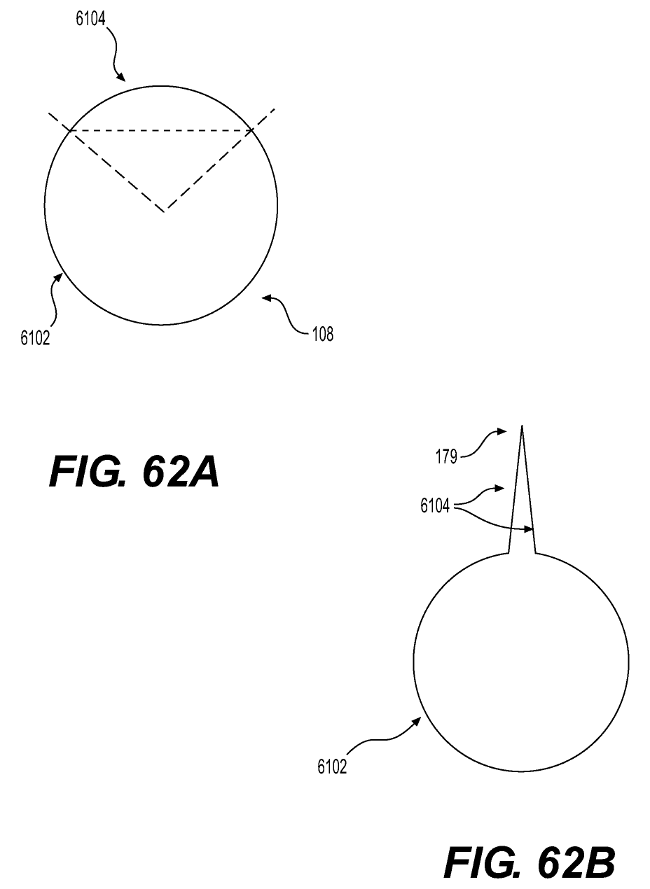

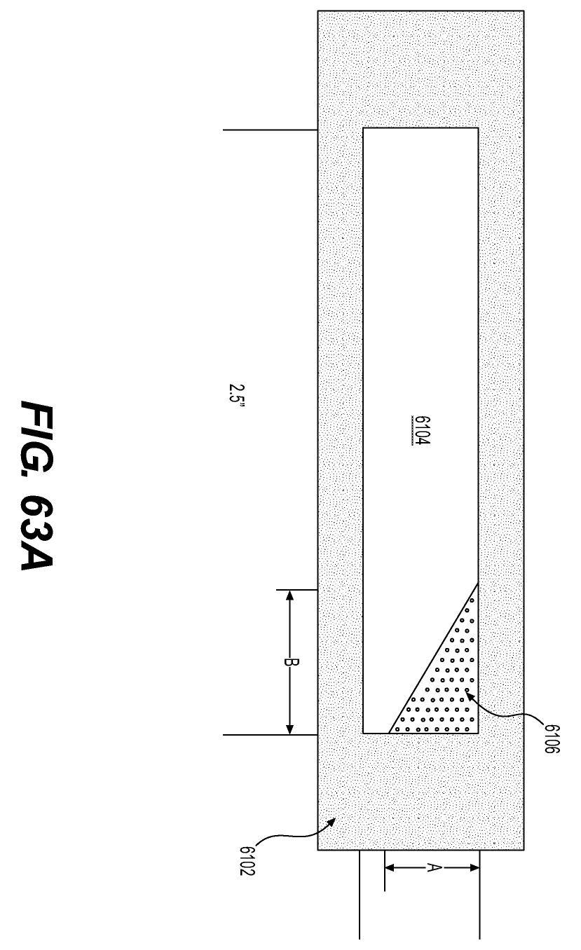

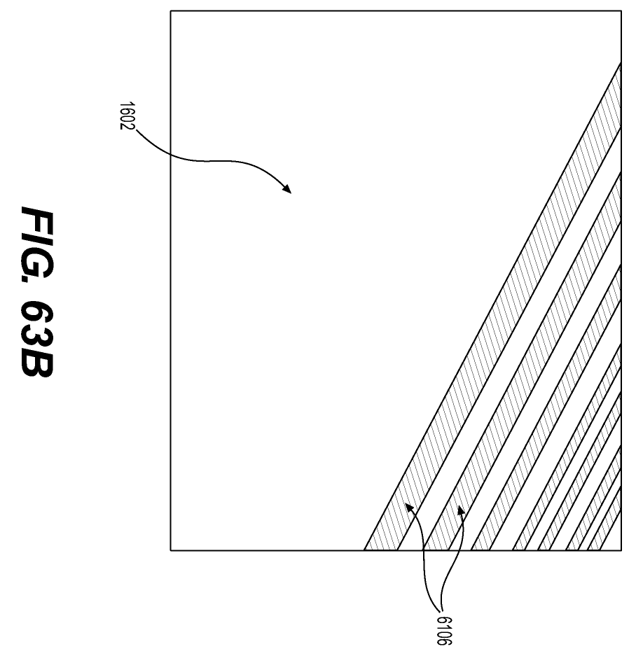

As described above, the sheath 100 includes an inner liner 108. Various exemplary aspects of the inner liner 108 (before the fold has been created) are shown in FIGS. 61-63. The liner 108 can comprise at least one first portion 6102, and at least one second portion 6104. In such aspects, the outer surface of the at least one first portion can have a composition that is substantially different from an outer surface of the at least one second portion. In yet further aspects, the outer surface of the at least one first portion can have a morphology that is substantially different from an outer surface of the at least one second portion. In such exemplary aspects, the at least one first portion 6102, can be etched, while the at least one second portion 6104 of the inner liner is unetched.

It is understood that the etching can be done by any known in the art methods. For example, and without limitation, the etching can be done by exposing at least a portion of the inner liner to an etchant. The choice of the specific etchant can be dependent on the composition of the inner liner itself. In certain aspects and as disclosed herein, the etchant can be a liquid, fluid, or gas, a plasma. In yet other exemplary aspects, the etchant can comprise sodium naphthalene, or it can comprise a plasma.

As described above, the inner liner 108 can comprise polytetrafluoroethylene (PTFE), fluorinated ethylene propylene (FEP), polyimide, polyetheretherketone (PEEK), polyurethane, nylon, polyethylene, polyamide, or combinations thereof. In yet further aspects, the inner liner comprises a fluorocarbon-based polymer, for example, PTFE and/or FEP. In aspects where the inner liner is a fluorocarbon, the etchant can be present as a solution, fluid, or gas. It can also be accelerated by applying elevated temperature, electricity, or voltage, or any combination thereof. Yet, in other aspects, the etchant can be a plasma. In such an exemplary aspect, the plasma etchant can be an oxygen plasma, hydrogen plasma, nitrogen plasma, argon plasma, or any combination thereof. It is understood that the degree of etching can be easily controlled by the desired masking, temperature of the solution, or power of the plasms, and the like.

In certain aspects, to form (or preserve) unetched portions of the inner liner, these portions can be masked first. It is understood that any masking methods can be utilized. In certain aspects, the masking can be performed by covering the portions that should not be etched with the materials that can withstand etching conditions without compromising the masked inner liner. In yet other aspects, the etching can be performed such that only the portions that are desired to be etched are exposed to the etchant.

In still further exemplary aspects, where the inner liner 108 comprises PTFE, at least a portion of this inner liner can be exposed to an exemplary etchant such as a sodium naphthalene solution. The exposure to the sodium naphthalene solution can result in a reaction between the sodium naphthalene and fluorine atoms present on the outer surface of the inner liner. In such exemplary and unlimiting aspects, the outer surface of the at least one first portion can comprise a fluorine-to-carbon ratio that is lower than a fluorine-to-carbon ratio of the unetched portions.

In yet further aspects, the etching can also affect a roughness of the outer surface of the inner liner in various portions. For example, the outer surface of the at least one first portion of the inner liner can exhibit an RMS value that is lower than an RMS value of the outer surface of the at least one second portion of the inner liner.

In yet further aspects, the etching can also affect the surface energy of the outer surface of the inner liner in various portions.

In yet further aspects, the etching can also affect the polarity of the outer surface of the inner liner in various portions.

In yet further aspects, the etching can also affect contact angle with a polar solvent like water on the outer surface of the inner liner.

In still further aspects, the inner liner 108 can be fully etched, and then some of the etched portions can be further surface-modified to form at least one second portion 6104 that is different from other etched (first) portions 6102. In such aspects, the at least one second portion can have the outer surface having a composition that is substantially different from a composition of the etched (first) portions. Similarly, there are also disclosed aspects, where the at least one second portion can have the outer surface having a morphology that is substantially different from a morphology of the etched (first) portions.

Various methods of surface modification can be utilized. In certain aspects, the surface modification can be done by exposing etched portions to the desired solution and chemically modifying the specific portions while masking the etched portions. The chemical surface modification can also be achieved by exposing the desired portions of the etched surface to a plasma treatment, for example. Yet, in other aspects, the surface modification can be a physical surface modification. It is understood that any known in the art physical surface modification can be utilized. In some aspects, the surface can be modified by the use of laser, for example, by laser ablation. While in other aspects, it can also be achieved by any method that results in the removal of the outermost layer of the outer surface of the inner liner.

In still further aspects, when for example, and without limitations, the laser ablation is utilized to surface-modify the etched first portions to form the second portions, such second portions can comprise a gradient morphology. However, it is also understood that the gradient morphology of the second portions is not limiting, and any shape of the structure of the ablating can be implemented. It is understood that a kerf of the laser can control the actual shape, for example. In yet other aspects, the shape of the ablation can be determined by the stability of the first portions (etched portions) of the inner liner. Some exemplary schematics of the gradient morphology 6106 are shown in FIGS. 63A-B. It is understood that the gradient morphology can have any predetermined pattern. In some exemplary and unlimiting aspects, the gradient can have a checkered, stripped, dotted, wavy, crisscross pattern, or any combination of the patterns depending on the desired utilization. It is understood that the gradient pattern can be controlled through a masking process allowing exposure to the laser ablation only in the desired locations.

In still further aspects, the gradient morphology can be controlled by laser power, frequency, and/or speed to arrive at the pattern having a desired shape, width, spacing between the liner (specific pattern), angle, directions, and/or depth of the lines. In certain aspects, the longitudinal lines can be formed with the laser ablation. In yet other aspects, and as shown in FIG. 63B, the lines formed by the laser ablation can have a different width or different angles.

In certain aspects, and as disclosed above, the at least one second portion, whether it is a completely unetched portion or it is the portion that was first etched and then surface-modified by any known in the art means, can extend longitudinally along a length of the inner liner. While in still further aspects, and as shown for example, in FIGS. 61A-B the at least one second portion, whether it is a completely unetched portion or the portion that was first etched and then surface-modified by any known in the art means, can extend circumferentially around a circumference of the inner liner.

In some exemplary aspects, the first and/or the second portions of the inner liner can have any shape. It is understood that in some exemplary and unlimiting aspects, the shape of the etched, unetched, or etched, and then surface-modified portions can be square, rectangular, rhombic, polygonal, trapezoid, pyramid, circular, oval, triangular, star, and the like. It is understood that in certain aspects, the second portion can have a combination of the unetched surface of the inner liner and etched and then the surface-modified surface of the inner liner. For example, the second portion can have an edge having any shape that has a gradient morphology, while the rest of the second section is unetched.

In still further aspects, the at least one second portion of the inner liner can extend along the inner portion and/or the outer portion of the at least one folded portion of the inner liner. In such aspects, the portion of the inner liner that is prepared to form a fold in the final sheath configuration can stay unetched, for example, to form second portions along the length of the inner liner forming a fold. The examples of forming such unetched portions that can form a future fold are shown in FIGS. 61A-B. In such exemplary aspects, the portion of the inner liner that forms the fold and crease 179 can be masked to stay unetched.

Yet in still further aspects and as described herein, the outer surface of the inner liner can be fully etched and then some of the portions of the outer surface are surface-modified by the laser ablation, for example, to form second portions that are substantially different from the etched (first) portions of the inner liner.

In still further aspects and as disclosed herein, the inner liner can have a wall thickness. In such exemplary aspects, the wall thickness of the inner liner can be from about 0.002 inches to about 0.006 inches, including exemplary values of about 0.0025 inches, about 0.003 inches, about 0.0035 inches, about 0.004 inches, about 0.0045 inches, about 0.005 inches, and about 0.0055 inches.

In yet further aspects, the at least one first portion has a wall thickness that is different from a wall thickness of the at least one second portion. In such exemplary aspects, where, for example, the at least one second portion is unetched portion of the inner liner, the wall thickness of this portion is up to about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or up to about 2% larger than the wall thickens of the at least one first portion.

While in still further exemplary aspects, where, for example, the inner liner is fully etched and the at least one second portion is then formed by a surface-modification of the desired etched portions of the inner liner, the wall thickness of this portion is up to about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or up to about 2% smaller than the wall thickness of the at least one first portion.

As discussed in detail herein, the inner liner can comprise two or more of the first portions and/or second portions. It is understood that in the aspects where the two or more of the first portions and/or second portions are present, each of the first portions can have the same or different wall thickness, and each of the second portions can have the same or different wall thickness.

It is even further understood that if, for example, two or more second portions are present, each of these portions can comprise either unetched material or etched and then surface-modified material.

In certain aspects and as shown in FIGS. 61A-B, for example, the first portion can be followed by the second portion that can be then followed by an additional first portion. It is understood that each of the two or more first portions, for example, can have the same or different length. Similarly, each of the two or more second portions can have the same or different lengths. For example, and without limitation, and as shown in FIG. 61B, the first portion 6102, can have a length of about 1.5 inches starting at the proximal end of the sheath. This first portion 6102 can then be followed with the second portion 6104 having an exemplary and unlimiting length of about 2.5 inches and followed again by the additional first portion having a much larger length. Again, it is understood that this depiction is only exemplary, and any other combination of the first and second portions having any desired lengths can be present. For example, and without limitation, the second portion can be located on the proximal end of the sheath, followed by the first portion and so on.

In still further aspects, the at least one second portion, so the inner liner extends along portions of the sheath that contact the outer surface of the outer layer. It is understood that the at least one second portion of the inner liner is in contact with the outer layer around the fold of the sheath. It is further understood that having these second portions improves sheath performance by reducing the stickiness to the outer layer, for example, making an unfolding process during the passage of the medical device easier. As disclosed in detail above, the outer layer can comprise any known in the art polymers. In certain aspects, the outer layer can comprise a high-density polyethylene (HDPE), nylon, and/or polypropylene polymer.

In still further aspects, the at least one second portion of the inner liner can exhibit substantially less stickiness to the outer layer as compared to the stickiness of the at least one first portion of the inner liner to the outer layer.

In still further aspects, the outer surface of the at least one second portion exhibits a contact angle from greater than about 65° to about 140°, including exemplary values of about 70°, about 75°, about 80°, about 85°, about 90°, about 95°, about 100°, about 105°, about 110°, about 115°, about 120°, about 125°, about 130°, and about 135°. While in yet other aspects, the outer surface of the at least one first portion exhibits a contact angle from about 30° to about 90°, including exemplary values of about 35°, about 40°, about 45°, about 50°, about 55°, about 60°, about 65°, about 70°, about 75°, about 80°, and about 85°.

In still further aspects, and as shown in FIG. 57, the sheath can also comprise an outer jacket 140. In such aspects, the outer jacket 140 can comprise an inner surface and outer surface and a proximal end and a distal end and extend at least partially around the outer layer such that the inner surface of the outer jacket overlies the outer surface of the outer layer. In some aspects, the outer jacket can have a proximal end and a distal end. In certain aspects, the proximal end of the outer jacket is abutted to the proximal end of the sheath. Yet, in other aspects, the distal end of the outer jacket can be anywhere along the length of the sheath. While in some exemplary aspects, the distal end of the outer jacket can abut the distal end of the sheath.

In certain aspects, where the outer jacket is present the at least one second portion of the inner liner is positioned at least around the distal end of the outer jacket. In such an exemplary aspect, as shown in FIG. 63A, for example, the second portion can have both unetched surface 6104 and gradient surface 6106. In this specific and unlimiting example, the gradient portion of the second portion is in a protruding area, or in other words, in the area where the outer jacket overlaps with the outer layer of the sheath.

Also disclosed herein are sheaths where the inner liner comprises two or more folded portions. In such aspects, it is understood that the at least one second portion of the inner liner can also extend along the inner portion and/or the outer portion of each of the two or more folded portions.

Also disclosed herein are aspects referring to the sheaths having a reduced insertion force as compared to any other available commercial sheaths. As one of ordinary skill in the art would readily appreciate, the reduction in the insertion force of the sheath and any medical device passing through results in reduced or substantially eliminated danger to the patients during the medical procedure. It is understood that the insertion force of the inventive sheaths, reference sheaths, and any other commercially available sheaths is measured using the same standardized technique for a proper comparison. In such aspects, a tensile tester, Instron 3366, can be used to measure a simulated insertion force that is required for a prosthetic valve to enter the described sheath. An additional constriction tube can be used to simulate vascular elasticity that contributes to push force. In certain exemplary aspects, the test can be performed in a water bath in a temperature range from room temperature to a normal body temperature (for example and without limitation from about 20° C. to less than about 40° C.). The specimen can be kept in a straight configuration. The test can be performed with a tapered mandrel to simulate a valve entering the sheath.

The sheaths as described herein that comprise one or more first and second portions exhibit a push force upon passing of the prosthetic device of at least about 10% lower, at least about 20% lower, at least about 30% lower, at least about 50% lower than a substantially identical reference sheath that does not comprise the second portion. Still, in further aspects, the sheaths as described herein and that comprise one or more first and second portions exhibit a push force upon passing of the prosthetic device of less than about 50 N, less than about 45 N, or less than about 40 N, less than about 35 N.

In still further examples, and as discussed above, sheath 100, unetched portions or etched and then surface-modified portions (second portions) can be provided along those surfaces of the inner liner 108 that come into contact with the outer surface of the outer layer 110. In the exemplary aspects where the tie layer is also present, like 128, those portions of the inner liner 108, excluding the tie layer 128, would not include etching. For example, it is contemplated that etching is not included between the inner surface of the folded portion 118 of the inner liner 108 and the underlying portion 122 of the outer layer 110. By excluding etching on the portions where the inner liner 108 and the outer surface of the outer layer 110 are in direct contact helps to facilitate the release of the inner surface of the folded portion 118 and the outer layer 110 during the expansion of the sheath 100. In yet further exemplary aspects, it is contemplated that portions that were etched and then surface-modified to form the second portions, as discussed above, are included between the inner surface of the folded portion 118 of the inner liner 108 and the underlying portion 122 of the outer layer 110. By including these disclosed second portions where the inner liner 108 and the outer surface of the outer layer 110 are in direct contact helps to facilitate the release of the inner surface of the folded portion 118 and the outer layer 110 during the expansion of the sheath 100.

Again as disclosed above, the wall thickness of the inner liner can vary, but in some examples, the wall thickness of the inner liner 108 ranges between about 0.002 inches and about 0.006 inches (including about 0.002 inches, about 0.003 inches, about 0.004 inches, about 0.005 inches, about 0.006 inches). In other examples, the wall thickness of the inner liner ranges between about 0.003 includes and about 0.005 inches. In a further example, the wall thickness of the inner liner 108 ranges between about 0.0035 inches and about 0.0045 inches (including about 0.0035 inches, about 0.0040 inches, about 0.0045 inches).

As described above, the sheath 100 includes an outer layer 110 exerting a radially inward force on the inner liner 108. In general, the outer layer 110 can comprise a polymeric material. As described above, the outer layer 110 can be comprised of PTFE, polyimide, PEEK, polyurethane, nylon, polyethylene, polypropylene, polyamide, polyether block amides, polyether block ester copolymer, thermoset silicone, latex, poly-isoprene rubbers, high density polyethylene (HDPE), Tecoflex™, or combinations thereof. In an exemplary aspect, the inner liner 108 can comprise PTFE, and the outer layer 110 can comprise a combination of HDPE and Tecoflex™. The outer layer 110 can have a wall thickness ranging between about 0.007 inches and about 0.013 inches (including 0.007 inches, about 0.008 inches, about 0.009 inches, about 0.010 inches, about 0.011 inches, about 0.012 inches, about 0.013 inches). In another example, the outer layer 110 can have a wall thickness ranging between about 0.008 inches and about 0.012 inches. In another example, the outer layer 110 can have a wall thickness ranging between about 0.009 inches and about 0.011 inches.

As described above, the sheath 100 includes an outer jacket 140 that extends over and envelopes the outer layer 110. While the outer layer 110 can be discontinuous, in that it includes a slit or a cut in order to form the overlapping and underlying portions 120, 122 as described above, the outer jacket 140 can include a continuous outer layer covering the inner and outer layers 108, 110.

The outer jacket 140 can be constructed from an elastomeric material such as a soft polymer material having good elasticity while also being abrasion- and tear-resistant. Generally, the outer jacket 140 has relatively low tensile modules compared to the inner and outer layers 108, 110. Example materials for the outer jacket 140 include, for example, a polyether block amide such as PEBAX®, or a thermoplastic polyurethane such as Neusoft® and Tecoflex™ 80A B20. In some examples, the outer jacket 140 comprises an elastomer (e.g., an elastic polymer) with shore hardness (durometer) ranging between about 10 and about 95 Shore A. The outer jacket 140 can comprise an elastomer with an elongation at break of ranging between about 40% and about 800% (including about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 800%). The outer jacket 140 can comprise an elastomer with a wall thickness ranging between about 0.003 inches and about 0.015 inches (including about 0.003 inches, about 0.004 inches, about 0.005 inches, about 0.006 inches, about 0.007 inches, about 0.008 inches, about 0.009 inches, about 0.010 inches, about 0.011 inches, about 0.012 inches, about 0.013 inches, about 0.014 inches, about 0.015 inches). The wall thickness is measured radially between the inner surface of the outer jacket 140 and the outer surface of the outer jacket 140.

In some examples, the outer jacket 140 comprises a single material or combination of materials having a constant thickness along the entire length of the outer jacket 140. In alternative examples, the material composition and/or wall thickness can change along the length of the outer jacket 140. For example, the outer jacket 140 can be provided with one or more segments, where the composition and/or thickness changes from segment to segment. In an example aspect, the Durometer rating of the composition changes along the length of the outer jacket 140 such that segments near the proximal end comprise a stiffer material or combination of materials, while segments near the distal end comprise a softer material or combination of materials. Similarly, the wall thickness of the outer jacket 140 the wall thickness of the outer jacket 140 in segments near the proximal end can be thicker/greater than the wall thickness of the outer jacket 140 near the distal end.

As illustrated in FIG. 35, the sheath 100 can include a tapered segment adjacent the flared end portion 114 at the proximal end of the sheath 100. Referred to as a strain relief section, the tapered segment and the flared end portion 114 help ease the transition between the smaller diameter portion of the sheath 100 and the housing 101. The thickness and/or composition of the outer jacket 140 can be adjusted to increase the Durometer and/or stiffness along the strain relief section. Because this portion of the sheath 100 is usually outside of the patient's body during the procedure, providing the outer jacket 140 with an increased Durometer and/or stiffness along the strain relief section helps to withstand the blood pressure that would otherwise cause the outer jacket 140 to “balloon up” with body fluid/blood. As a result, it allows for a sheath 100 having a relatively stiff proximal end at the point of introducing a delivery apparatus while still having a relatively soft distal tip at the point of entry into the patient's vessel.

The outer jacket 140 can be bonded to the outer layer 110 to prevent the outer jacket 140 from sliding over the outer layer 110 and “bunching up” in response to the friction forces applied by the surrounding tissue during insertion of the sheath 100 into the patient's vasculature. For example, the outer jacket 140 can be bonded at the proximal end and/or distal end of the outer layer 110. At the proximal and distal ends, the outer jacket 140 can be bonded to the outer layer 110 around the full circumference of the outer layer. At the distal end of the sheath 100, the outer jacket 140 can alternatively be bonded to the inner liner 108. For example, the outer jacket 140 can be bonded to the distal end surface of the inner liner 108.

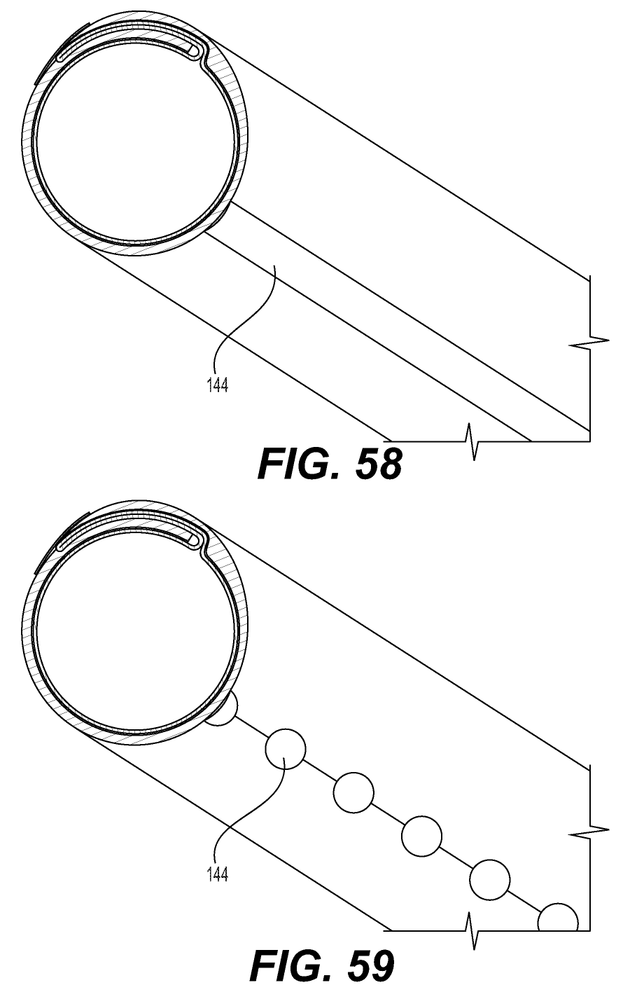

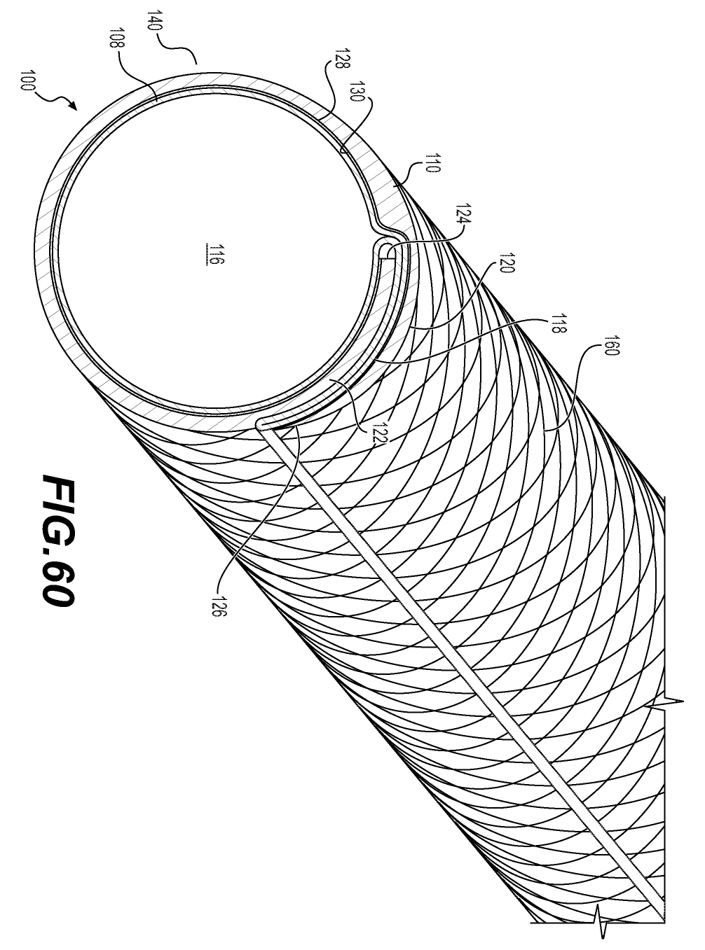

As illustrated in FIG. 57, the outer jacket 140 can be bonded 144 to the outer layer 110 at a circumferential location opposite the folded portion of the inner liner 108. As provided in FIGS. 58-59, the bond 144 can be spot bonds or linear bond lines extending along all or a portion of the outer layer 110. As provided in FIG. 57, the bond 144 line/spot will also have a width, extending circumferentially around the outer layer 110. For example, the bond line can cover about 5° to about 90° of the circumference of the outer layer 110 (including about 5°, about 10°, about 15°, about 20°, about 25°, about 30°, about 35°, about 40°, about 45°, about 50°, about 55°, about 60°, about 65°, about 70°, about 75°, about 80°, about 85°, about 90°).

The outer jacket 140 can be bonded to the outer layer 110 and/or inner liner 108 using any mechanical and/or chemical (e.g., adhesive) fastener known in the art. In one example, sheath 100, the outer jacket 140 and the outer layer 110 and/or inner liner 108 may have similar melting temperatures. Accordingly, an example bonding method includes a thermally bonded coupling between the outer jacket 140, the outer layer 110, and/or inner liner 108. For example, the bond between the outer jacket 140 and the outer layer 110 can be achieved by laser welding and/or a heat compression (e.g., using a heat compression jaw), allowing the location of the bond line to be closely controlled.

As shown in FIG. 54, and described above with respect to FIG. 39, an adhesive layer 128 (e.g., a tie layer) is provided between the inner liner 108 and the outer layer 110 to at least partially adhere the inner liner 108 to the outer layer 110. In some aspects, the adhesive layer 128 can be selectively provided/located between the inner liner 108 and the outer layer 110 to bond the inner and outer layers 108, 110 at the selected locations of the adhesive layer 128.

As illustrated in FIG. 54 (and FIG. 39) the adhesive layer 128 is provided on the outer surface of the inner liner 108 and/or the inner surface 130 of the outer layer 110. For example, the adhesive layer 128 can be provided partially or entirely around the outer surface of the inner liner 108. Additionally, or alternatively, the adhesive layer 128 can be provided partially and/or entirely around the inner surface 130 of the outer layer 110. As illustrated in FIG. 54, the adhesive layer 128 extends between the outer layer 110 and the overlapping folded portion 118 of the inner liner 108. In other aspects, the adhesive layer 128 can extend between the outer surface of the folded portion 118 of the inner liner 108 and the corresponding inner surface of the overlapping portion 120 of the outer layer 110. As illustrated in FIG. 54, the adhesive layer 128 does not extend between an inner surface of the overlapping folded portion 118 of the inner liner 108 and a corresponding surface of the underlying portion 122 of the outer surface of the outer layer 110. Excluding the adhesive layer 128 on the portion of the sheath between the inner surface of the folded portion 118 and the underlying portion 122 facilitates expansion of the sheath and prevents undesirable bonding/sticking between the inner and outer layers 108, 110 at this location.

The adhesive material 128 can comprise a material having a Shore A hardness (durometer) less than about 90A. For example, the adhesive material 128 can comprise a thermoplastic polyurethane such as an aliphatic polyether-based thermoplastic polyurethane (TPU). An example of TPU includes Tecoflex™ 80A. The adhesive layer 128 can also be composed of an aromatic polyether or polyesters based thermoplastic polyurethane such for example, Pellethane™ 80A. The adhesive layer can also be composed of a polyolefin or polyamide, including, for example, a polyolefin (PE, PP, or EVA) modified with maleic anhydride such as an Orevac™ resin.

The thickness (wall thickness) of the adhesive layer 128 can vary, but in some examples, the wall thickness of the adhesive layer ranges between about 0.002 inches and about 0.005 inches (including about 0.002 inches, about 0.003 inches, about 0.004 inches, about 0.005 inches). In other examples, the wall thickness of the adhesive layer 128 ranges between about 0.0025 and about 0.0040 (including about 0.0025 inches, about 0.0030 inches, about 0.0035 inches, about 0.0040 inches). In a further example, the wall thickness of the adhesive layer 128 ranges between about 0.0025 inches and about 0.0035 inches (including about 0.0025 inches, about 0.0030 inches, about 0.0035 inches).

In some examples, the sheath 100 can include a lubricant to reduce friction and facilitate expansion/contraction between the outer layer 110 and the outer jacket 140. The lubricant 142 allows the outer layer 110 and inner liner 108 to unroll easily under the outer jacket 140, ensuring that the hemostasis and atraumatic benefits achieved by the addition of an outer jacket 140 does not comprise the push force performance of the sheath 100. I some aspects, the lubricant 142 can reduce the push force necessary to move the prosthetic device through the central lumen 116 of the inner liner 108 during delivery of the prosthetic device and the corresponding local expansion of the sheath 100.

As illustrated in FIG. 56, the lubricant 142 can be selectively applied along an outer surface of the outer layer 110 proximate the longitudinally extending edge 126 of the overlapping portion 120. In some examples, a portion of the folded portion 118 of the inner liner 108 extends beyond the longitudinally extending edge 126 of the overlapping portion 120 and along an outer surface of the outer layer 110. In this example, the lubricant 142 is also provided along the protruding portion of the folded portion 118 of the inner liner 108 extending along the outer surface of the outer layer 110 (beyond the edge 126). In this location, the lubricant 142 also reduces friction between the outer jacket 140 and the inner liner 108 during expansion of the sheath 100. As illustrated in FIG. 56, the lubricant 142 extends around the circumference of the outer layer 110 beyond the protruding portion of the folded portion 118.

The lubricant is applied as a band (or spot) that extends both circumferentially and longitudinally along the outer layer 110 (and the protruding portion of the inner liner 108). In an example sheath 100, the lubricant 142 is applied as a band that extends both circumferentially around the outer layer 110 and longitudinally along a length of the outer layer 110. To prevent migration, the lubricant 142 can be composed of a heat-curable material, e.g., a material curable at room temperature. As a result, the material can be applied to a desired location along the outer layer 110 and does not migrate during assembly and/or use of the sheath 100. The lubricant 142 can be composed of a medical-grade lubricant, such as silicone. Example lubricants include medical-grade curable silicone lubricants, including a platinum-catalyzed thermal curing silicone lubricant such as NuSil™ MED10-6670 (heat-curable), a PTFE lubricant such as Duraglide™ (curable at room temperature) and/or CHRISTO-LUBE™.

FIG. 60 illustrates an additional aspect of the sheath 100 of FIG. 35. In this example, the sheath 100 can include a coiled wire 160, or coiled wire mesh, along a length of the sheath 100. The coiled wire 160 provides uniform bending of the sheath and prevents kinking. The coiled wire 160 can be embedded in the outer layer b. For example, the coiled wire 160 can be co-extruded with the outer layer 110. Alternatively, the coiled wire 160 can be provided between the outer layer and the adhesive layer 128. In another example, the coiled wire 160 is embedded, at least partially, within both the outer layer 110 and the adhesive layer 128. For example, the coiled wire 160 can be provided on an outer surface of the adhesive layer 128, and the outer layer 110 is reflowed over.

As illustrated in FIG. 60, the coiled wire 160 defines a helical-shaped path around the longitudinal axis of the sheath 100. The example of coiled wire 160 includes an overlapping helical-shaped path around the longitudinal axis of the sheath 100, resulting in a continuous diamond-shaped pattern along the length of the sheath.

The coiled wire 160 can be comprised of a metal or a polymer wire. For example, the coiled wire 160 can be composed of PET, PEEK, stainless steel, and/or nitinol. The coiled wire 160 can be comprised of a flat wire, a round wire, or a combination thereof. The individual wires of the coiled wire 160 can have a diameter/thickness ranging between about 0.002 inches and about 0.008 inches (including about 0.002 inches, about 0.003 inches, about 0.004 inches, about 0.005 inches, about 0.006 inches, about 0.007 inches, about 0.008 inches). In another example, the individual wires of the coiled wire 160 can have a diameter/thickness ranging between about 0.004 inches and about 0.007 inches. In a further example, the individual wires of the coiled wire 160 can have a diameter/thickness of about 0.006 inches. The pitch/distance between adjacent coils of the coiled wire 160 can correspond to the diameter/thickness of the coiled wire. For example, where the diameter/thickness of a single coil of the coiled wire is about 0.006 inches, the spacing/pitch between the wire and the next adjacent coiled wire is about 0.006 inches.

Methods of using the sheath of FIGS. 53-60 include first inserting the expandable sheath 100 into the vasculature of a subject and advancing a prosthetic device through the inner lumen 116 of the inner liner 108/sheath 100. The prosthetic device applies an outwardly directed radial force on the inner liner 108 of the expandable sheath 100. In some aspects, the outwardly directed radial force is transmitted through the inner liner 108, the adhesive layer 128, and the outer layer 110. The lumen 116 of the sheath 100 expands at the axial location of the prosthetic device due to the outwardly directed radial force exerted by a prosthetic device against an inner surface of the lumen during advancement. During expansion of the lumen 116, the first fold (folded edge) of the folded portion 118 is moved circumferentially closer to the second fold (folded edge), shortening the overlapping portion of the folded portion 118 that extends circumferentially between the first and second folds, thereby increasing the circumference of the lumen 116.

As the sheath 100 expands at a particular location (i.e., locally expands at the location of the passing prosthetic device), the overlapping portion 120 of the outer layer 110 can move circumferentially with respect to the underlying portion 122 as the folded portion 118 of the inner liner 108 least partially separate and/or unfold, causing the elongate gap(s) 132 provided in the outer layer 110 to widen/expand. The sheath thereby expands to accommodate a medical device having a diameter larger than that of lumen 116 in the resting (unexpanded) configuration. As shown in FIG. 55, in some aspects, the folded portion of the inner liner 108 can completely unfold so that the inner liner 108 forms a cylindrical tube at the location of the expanded configuration. As illustrated in FIGS. 54 and 55, the elongate gap 132 is generally aligned with the longitudinal axis of the lumen 116 such that during expansion, the unfolded portion of the inner liner 108 expands into the gap 132.

In an unexpanded configuration, the sheath 100 can have an outer diameter less than about 0.030 inches (including less than about 0.029 inches, less than about 0.028 inches, less than about 0.027 inches, less than about 0.026 inches, less than about 0.025 inches, less than about 0.024 inches). Preferably, the unexpanded sheath has an outer diameter ranging between about 0.024 inches and about 0.026 inches. In a fully expanded configuration, the sheath 100 can have an inner diameter greater than 0.040 inches. Preferably, the expanded sheath 100 has an inner diameter ranging between 0.046 inches and 0.054 inches (including about 0.046 inches, about 0.047 inches, about 0.048 inches, about 0.049 inches, about 0.050 inches, about 0.051 inches, about 0.052 inches, about 0.053 inches, about 0.054 inches)

As described above, the inner and outer layers 108, 110 can be bonded together using an adhesive layer 128. The adhesive layer 128 prevents movement, both longitudinal and radial, between the inner and outer layers 108, 110. As a result, expansion of the sheath 100 can be limited to only those regions excluding the adhesive layer 128. For example, as illustrated in FIG. 54, because the adhesive layer 128 is not provided between the inner surface of the folded portion 118 and the underlying portion 122 of the outer layer, expansion of the sheath results in the inner surface of the folded portion extending into the gap 132 created between the first and second edges 124, 126 of the expanded outer layer 110.

Once the prosthetic device is passed through the lumen 116 (or a particular location along the lumen), the lumen 116 of the sheath can at least partially contracts back to an unexpanded configuration. The outer layer 110 can exert an inwardly directed radial force on the inner liner 108 urging it back to its original folded configuration. Similarly, if a coiled wire 160 was included, the coiled wire can exert an inwardly directed radial force on the outer layer 110 and the inner liner 108 urging them back towards an unexpanded configuration. Likewise, if an outer jacket 140 is included, the outer jacket 140 can exert an inwardly directed radial force on the outer layer 110 and the inner liner 108 urging them back to an unexpanded configuration.

The prosthetic device can be delivered through the distal end of the sheath 100 to the delivery site within the patient. The prosthetic device can include a self-expanding heart valve or a stent-mounted heart valve. The heart valve can be extended through the distal end of the elongate lumen 116 at the delivery site. Once outside the lumen 116, the heart valve can be expanded, and the sheath 100 removed from the treatment site.

An example method of making the sheath is as follows. These steps are not meant to be limiting. The steps given can be reordered as needed. Other steps can be added, or in other examples, some steps may not be necessary. A continuous inner liner 108 defining a lumen therethrough is provided. The inner liner 108 is formed to include a first fold and a second fold and an overlapping folded portion 118 extending circumferentially between the first and second folds. The overlapping folded portion 118 is formed to include overlap in a radial direction of at least two thicknesses of the inner liner 108. The inner liner 108 can be extruded, including the folded portion 118. Alternatively, the folded portion 118 can be formed after the inner liner 108 is extruded (e.g., formed on a cylindrical tubular structure).

A discontinuous outer layer 110 is provided (at least partially) around the inner liner 108. The outer layer 110 is formed to include an overlapping portion 120 and an underlying portion 122 such that at least a portion of the folded portion 118 of the inner liner 108 is positioned between the overlapping portion 120 and the underlying portion 122. In some aspects, the inner liner 108 and the outer layer 110 are coextruded. In alternative aspects, the inner liner 108 and the outer layer 110 are separately formed and joined together.

An adhesive layer 128 is provided between the inner liner 108 and the outer layer 110 for bonding (at least partially) the inner liner 108 to the outer layer 110. The adhesive layer 128 can be applied to and bond the inner and outer layers 108, 110 axially along a length of the sheath, e.g., along a portion of the entire length of the sheath or along the entire length of the sheath. In an example, the adhesive layer 128 is coextruded with the outer layer 110. In a further example, the adhesive layer 128 is coextruded with the inner liner 108. In an alternate example method, the adhesive layer 142 can be applied to the outer surface of the inner liner 108 and/or an inner surface of the outer layer 110.

After the adhesive layer 128 is applied to the desired locations along the inner and/or outer layer 110, the outer layer 110 is applied over the inner liner 108. The outer layer 110 can then be bonded to the inner liner 108 by heat curing the adhesive layer 128. The adhesive layer 128 can comprise a material curable at a temperature above room temperature, in which case a heat treatment may be applied to the assembled inner liner 108/outer layer 110. The adhesive layer 128 may also be composed of a material that cures at room temperature. Accordingly, after application of the adhesive layer 128 and assembly of the outer layer 110 over the inner liner 108 (at a temperature below room temperature), the temperature of the combined layers may be increased to room temperature.

As illustrated in FIG. 56, the lubricant 142 can be selectively applied to the desired location along a length of outer layer 110 and/or outer jacket 140. As described above, the lubricant 142 can be provided on an outer surface of the outer layer 110 proximate a longitudinally extending edge 126 of the overlapping portion 120, on any portion of the folded portion 118 extending/protruding beyond edge 126, and/or on any portion of the outer surface of the outer layer 110 adjacent the protruding portion of the folded portion 118. The lubricant 142 can be applied as a band extending around a portion of the circumference of the outer layer, the band of lubricant 142 also extending longitudinally along a length of the outer layer 110. After the lubricant 142 is selectively applied to the outer surface of the outer layer 110, the outer jacket 140 can be applied over the outer layer 110. As outlined above, the lubricant 142 can comprise a heat-curable material, in which case a heat treatment can be applied to the outer layer 110/outer jacket 140. The lubricant 142 may also be composed of a material that cures at room temperature. Accordingly, after application of the lubricant 142 (at a temperature below room temperature), the temperature of the outer layer 110 (separately or in combination with the outer jacket 140) can be increased to room temperature.

The outer jacket 140 can then be applied over/around the outer layer 110 and bonded to the outer layer 110 at at least one of the proximal and distal ends of the outer layer 110. The outer jacket 140 can also be bonded to the outer layer 110 along a length of the outer layer 110. The outer jacket 140 can be bonded to the outer layer 110 via a heat treatment process, e.g., a reflow process where the outer layer 110 and the outer jacket are headed to a temperature high enough such that the outer layer 110 and the outer jacket 140 are at least partially melted and are then fused together are the heat is removed, and the assembly cools. The entire sheath 100 assembly may be reflowed to reduce the overall outer diameter and regain/ensure a circular shape in cross-section.

As described above, select portions of the outer surface of the inner liner 108 can include a surface treatment such as surface etching. In an example method, surface treatment of the inner liner 108 would occur before application of the outer layer 110. It is contemplated that it may be desirable to exclude etching from those surfaces of the inner liner 108 that come into contact with the outer surface of the outer layer 110. For example, etching may not be included between the inner surface of the folded portion 118 of the inner liner 108 and the underlying portion 122 of the outer layer 110. By excluding etching on the portions where the inner liner 108 and the outer surface of the outer layer 110 are in direct contact helps to facilitate release of the inner surface of the folded portion 118 and the outer layer 110 during expansion of the sheath 100.

In some instances, it may be necessary to release (“break”) any undesirable bonding that occurs between the outer layer 110 and the inner liner 108. This bonding can occur due to the etching on the outer surface of the inner liner 108 that allows it to stick directly to the outer layer 110 (with/without the adhesive layer 128). Undesirable bonding can also occur if the outer layer 110 can flows on top of the folded inner liner 108 and “grabs” it during the reflow process.

However, when the inner liner 108 is unetched along those locations excluding the adhesive layer 128, e.g., those portions adjacent the underlying portion of the outer layer 110, undesirable bonding between the outer layer 110 and the inner liner 108 at this location is limited. Therefore, it may not be necessary to precondition the sheath to release undesirable bonding between the inner liner 108 and the outer layer 110 because the bonding has not occurred (or is less likely to occur).

Nonetheless, undesirable bonding between the inner liner 108 and the underlying portion 122 of the outer layer 110 can be released while maintaining desirable bonding at the proximal and distal ends of the sheath 100. For example, a mandrel can be passed at least partially through the lumen 116 of the inner liner 108, expanding the inner liner 108 and the outer layer 110, and breaking/releasing any undesirable bonding between the inner liner 108 and the underlying portion 122 of the outer layer 110.

FIGS. 41-49 illustrate additional aspects and variations on the general sheath 100 described above. It is to be understood that the variations (e.g., materials and alternate configurations) described above with reference to FIGS. 35-40 can also apply to the aspects shown in FIGS. 41-49. Furthermore, the variations described below with reference to FIGS. 41-49 can also be applied to the sheath described in FIGS. 35-40.

FIGS. 41-43 illustrate a sheath 700 that additionally includes a strain relief cover, also referred to as an elastic outer cover, or an elastic cover 702 positioned around at least a part of an inner liner 704 and outer layer 706. As shown in FIG. 41, the elastic cover 702 can extend for a length L along at least a portion of the main body of the sheath 700. In some aspects, the elastic cover 702 can extend from the proximal end 708 of the sheath 700 and towards the distal end 709 of the sheath. In some aspects, the elastic cover 702 extends only partway down the length of the sheath 700. In alternate aspects, the elastic cover 702 can extend to a point adjacent the distal end 709 or can extend all the way to the distal end 709 of sheath 700. Furthermore, the elastic outer cover 702 need not extend all the way to the proximal end 708 of the sheath 700. In some aspects, the elastic outer cover 702 may extend only partway towards the proximal end 708. In some aspects, the longitudinal length L of the elastic cover 702 can range from about 10 cm to the entire length of the sheath 700.

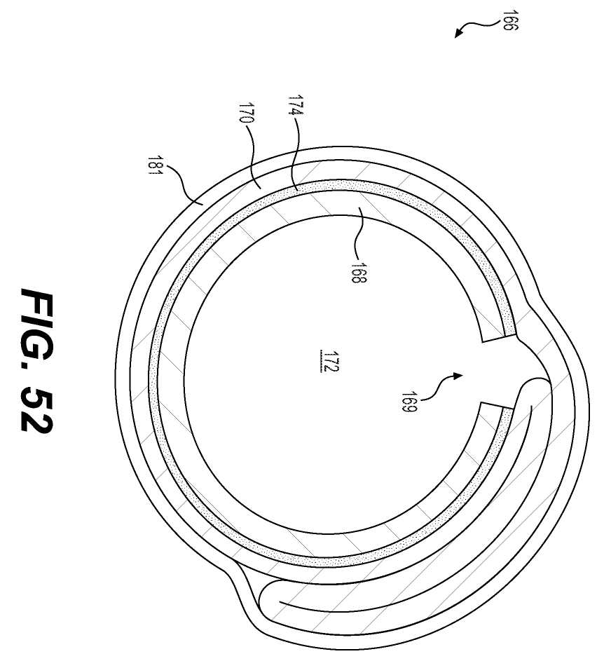

As shown in FIGS. 42 and 43, the elastic cover 702 can be a continuous tubular layer, without slits or other discontinuities. The elastic cover 702 can be positioned to surround the entire circumference of outer layer 706 and can extend longitudinally along any portion of the length of the sheath 700. The elastic outer cover 702 can comprise any pliable, elastic material(s) that expand and contract, preferably with a high expansion ratio. Preferably, the materials used can include low durometer polymers with high elasticity, such as PEBAX®, polyurethane, silicone, and/or polyisoprene. Materials for the elastic outer cover 702 can be selected such that it does not impede expansion of the sheath 700. In fact, the elastic outer cover 702 can stretch and expand as the sheath 700 expands, such as by movement of the folded or scored inner liner with respect to itself.

The elastic outer cover 702 can, in some aspects, provide hemostasis (e.g., prevent blood loss during implantation of the prosthetic device). For example, the elastic outer cover 702 can be sized or configured to form a seal with the patient's artery when inserted, such that blood is substantially prevented from flowing between the elastic outer cover 702 and the vessel wall. The elastic outer cover 702 can be inserted such that it passes the arteriotomy. For example, in aspects where the elastic outer cover 702 does not extend all the way to the distal end 709 of the sheath 700, the elastic cover 702 can extend distally far enough such that when the sheath 700 is fully inserted into the patient, at least part of the elastic outer cover extends through the ateriotomoy site.

The elastic outer cover can have a thickness ranging from, for example, about 0.001″ to about 0.010.″ In some aspects, the outer cover can have a thickness of from about 0.003″ to about 0.006.″ The elastic outer cover can be configured to expand as the sheath expands, as shown in the expanded configuration in FIG. 43.

FIG. 42 shows a cross-section of the sheath 700 in a resting configuration having an inner diameter D1. FIG. 43 shows a cross-section of the sheath 700 in an expanded configuration, having an inner diameter D2, where D2 is greater than D1. Similar to the aspect of FIGS. 35-40, the sheath 700 can include an inner liner 704 having a folded portion 710, and an outer layer 706 having an overlapping portion 712 and an underlying portion 714. The overlapping portion 712 overlaps at least a portion of the folded portion 710 of the inner liner, and the underlying portion 714 underlies at least a portion of the folded portion 710. As shown in FIGS. 42-43, in some aspects, the overlapping portion 712 does not overlap the entire folded portion 710 of the inner liner 704, and thus, a portion of the folded portion 710 can be directly adjacent to the elastic outer cover 702 in locations where the elastic cover 702 is present. In locations where the elastic cover 702 is not present, part of the folded portion 710 may be visible from the outside of the sheath 700, as seen in FIG. 41. In these aspects, the sheath 700 can include a longitudinal seam 722 where the overlapping portion 712 terminates at the folded portion 710. In use, the sheath can be positioned such that the seam 722 is posterior to the point of the sheath that is 180 degrees from the seam 722 (e.g., facing downward in the view of FIG. 41). The seam 722 can also be seen in FIG. 41, which shows that the seam 722 need not extend the entire length of the sheath. In some aspects, the proximal end portion of the sheath includes two layers without a folded portion (e.g., similar to FIG. 38), while the distal end portion of the sheath includes two layers with a folded portion (e.g., similar to FIG. 39). In some aspects, the seam 722 can end at a transition point between portions of the sheath having a folded inner liner and portions of the sheath not having a folded inner liner.

In some aspects, the folded portion 710 can include a weakened portion, such as a longitudinal perforation, score line, and/or slit 716 along at least a portion of the length of the inner liner 704. The slit 716 can allow for two adjacent ends 718, 720 of the folded portion 710 to move relative to one another as the sheath 700 expands to the expanded configuration shown in FIG. 43. As a device having an outer diameter device larger than the initial resting inner diameter of the sheath 700 is inserted through the sheath 700, the device can cause local expansion of the sheath 700 and cause the sheath 700 to expand at the partial score or split line location 716. The weakened portion 716 can extend longitudinally along any portion of the expandable sheath 700.

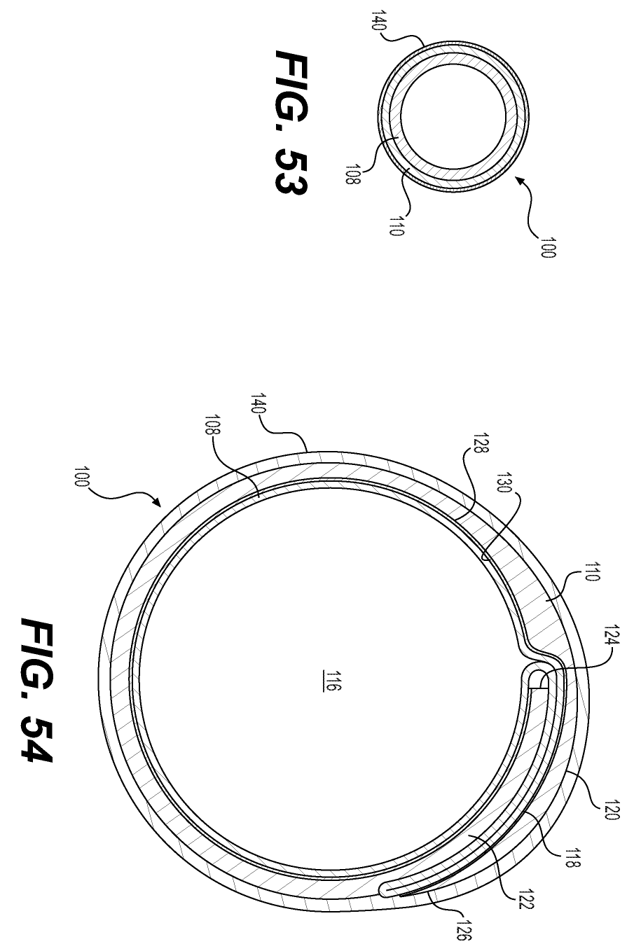

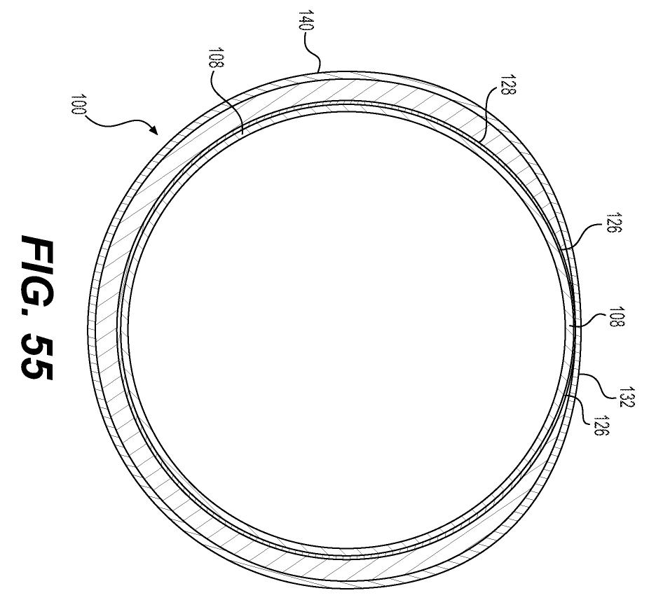

FIGS. 44 and 45 show another aspect of an expandable sheath 800 having an initial diameter in a resting configuration (FIG. 44) and a larger expanded diameter in an expanded configuration (FIG. 45). The sheath 800 can include an elastic outer cover 802, an inner liner 804, and an outer layer 806. Inner liner 804 can include first and second folded portions 808, 810. The folded portions 808, 810 can be arranged such that they fold away from one another in opposite directions around the circumference of the sheath 800. For example, folded portion 808 can be folded to the right in the view of FIG. 44 and folded portion 810 can be folded to the left such that they do not overlap one another but share a common segment 812, which is part of both folded portions 808, 810. In contrast to previous aspects, the outer layer 806 does not include an overlapping portion in this aspect but rather has first and second underlying portions 814, 816, which underlie the first and second folded portions 808, 810, respectively. The inner liner 804 can extend through a gap between the ends of the adjacent underlying portions 814, 816 (e.g., between a first end and a second end of discontinuous outer layer 806).

Each folded portion 808, 810 can include a weakened portion 818, such as a slit, score line, and/or perforation. Weakened portion 818 can allow the expandable sheath 800 to expand easily without a high radial force. As the sheath 800 expands, segment 812 along the top of the folded portions 808, 810 of inner liner 804 can be configured to split apart from the rest of the folded portions 808, 810 and the first and second underlying portions 814, 816 can move away from one another so as to create an enlarged lumen within the inner liner 804. Weakened portions 818 can allow for the segment 812 to easily split apart from the inner liner 804 as the sheath 800 expands.

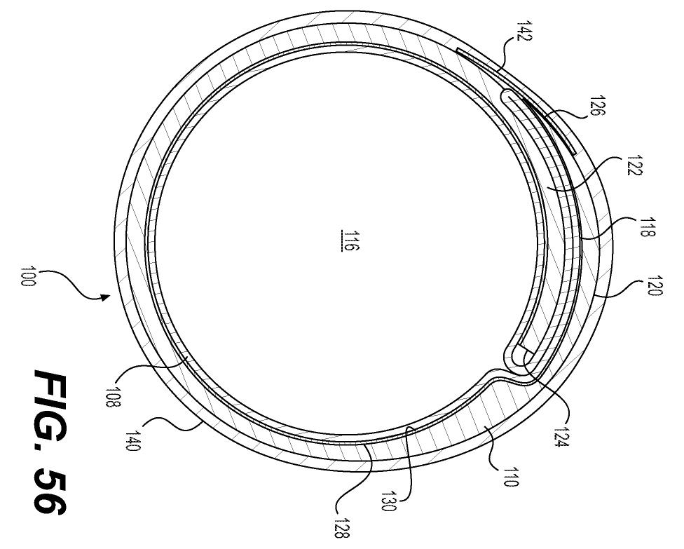

FIGS. 46-47 show another aspect of an expandable sheath 900. Sheath 900 can be provided with an inner liner 902 and an elastic cover 904 surrounding the inner liner 902. While not shown, sheath 900 can additionally include an intermediate layer positioned between the inner liner 902 and the elastic cover 904. If present, the intermediate layer can closely follow the contour of the inner liner 902.

Inner liner 902 can be shaped to include one or more folded portions 906 arranged to form a generally horseshoe-shaped lumen 908 that extends longitudinally through sheath 900 along the inner surface of the inner liner 902. The folded portions 906 can be arranged to form an area 910 positioned with the lumen 908 and radially inward from the elastic cover 904. In some aspects, the area 910 can include one or more voids (e.g., smaller lumens or openings extending through portion 910). In some aspects, the area 910 can be filled with material (e.g., HDPE) reflowed from an intermediate layer while the sheath is being made. In some aspects, the area 910 can be filled with material reflowed from the elastic cover 904 during the sheath manufacturing process.

The inner liner 902 can include one or more weakened portions 912, such as score lines, perforations, or slits. The weakened portions 912 can be configured to split apart, separate, or widen as the sheath expands from its initial resting configuration (FIG. 46) to an expanded configuration (FIG. 47) in the presence of a radial force. As the sheath 900 expands, material from the area 910 can cover any gaps 914 formed at the weakened portions 912, thereby keeping the lumen 908 substantially sealed.

FIG. 48 shows another aspect of an expandable sheath 1000 having an inner liner 1002 and a discontinuous outer layer 1004. Sheath 1000 is similar to the sheath 800 of FIG. 44, except that sheath 1000 is shown without an elastic outer cover, and further, the inner liner 1002 is continuous, without weakened portions at the folds 1006. As shown in FIG. 48, the inner liner 1002 can be configured to have one or more folds 1006 (e.g., two folds positioned on the outer surface of the outer layer 1004), with portions 1008 of the outer layer 1004 extending between the folds 1006 and the outer surface 1010 of the inner liner 1002 underlying the folds 1006.

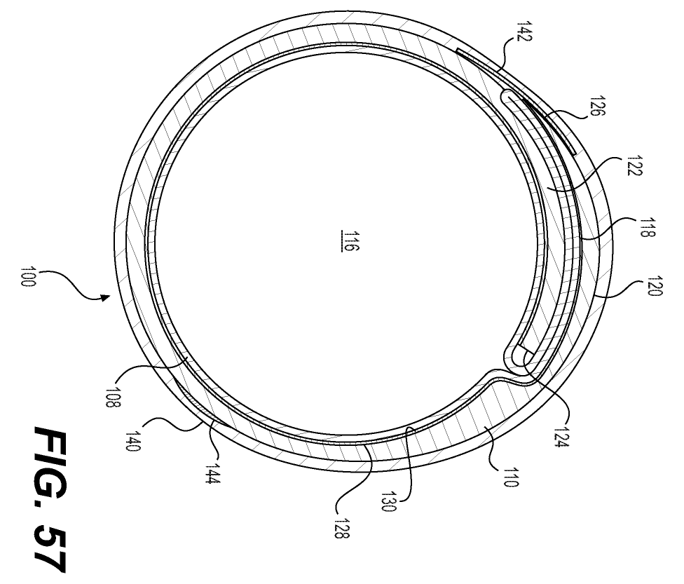

FIG. 49 shows another aspect of an expandable sheath 1100 having an inner liner 1102 and an outer layer 1104. The sheath 1100 is similar to the sheath 100 shown in FIG. 39 in that the inner liner 1102 can be continuous with a folded portion 1106, and the outer layer 1104 can be discontinuous with an overlapping portion 1108 overlapping at least a part of the folded portion 1106 and an underlying portion 1110 underlying at least a part of the folded portion 1106. The underlying portion 1110 can thus be positioned between an outer surface 1112 of the lumen-forming portion of the inner liner 1102 and the folded portion 1106.

The inner liners 1002, 1102 of the sheaths 1000, 1100, respectively, of FIGS. 48-49 can be optimized to perform slightly differently than the inner liners of sheaths described above. For example, different materials can be used for the inner liner to increase the durability and softness of the seam (although such materials can also be used with the other aspects of expandable sheaths described above). For example, materials such as woven fabrics or braid filaments can be used. Such fabrics, filaments, or yarns can comprise, for example, PTFE, PET, PEEK, and/or nylon yarns or filaments. These materials can advantageously provide a soft and flexible layer that can be easily formed into the desired shapes or folded portions. Additionally, such materials can withstand high temperatures, as well as can possess high tensile strength and tear resistance. Nonetheless, these materials can also be elastic, experience minimal kinking, and provide soft distal edges for less traumatic insertion into a patient's vessels.

Various methods can be used to produce the sheaths discussed above and below throughout the present disclosure. For example, a method of making the sheath shown in FIGS. 2A-2D can comprise providing a mandrel and applying an inner liner on the mandrel, such as by spray coating or dip coating the mandrel. An intermediate layer, such as a mesh structure, can then be mounted on the inner liner. An outer layer can be applied over the intermediate layer, such as by a second spray coating or dip coating step. Methods can comprise etching or surface treating at least a portion of the inner liner. Also, methods can comprise providing one or more notches and/or cuts in the inner liner and/or the outer layer. Cuts and/or notches can be provided by, for example, laser cutting or etching one or more layers.

In some aspects of methods of making a sheath, such as the sheaths illustrated in FIGS. 2A-2D, layers can be pre-formed and mounted on a mandrel and then fused or thermally bonded together. For example, in one method, an inner liner is applied to a mandrel. An intermediate layer can be applied to the outer surface of the inner liner. An outer layer can be applied to the outer surface of the intermediate layer. Heat shrink tubing can be applied and the assembly heated, such that the inner liner, the intermediate layer, and/or the outer layer are thermally bonded and compressed together under the heat shrink tubing.

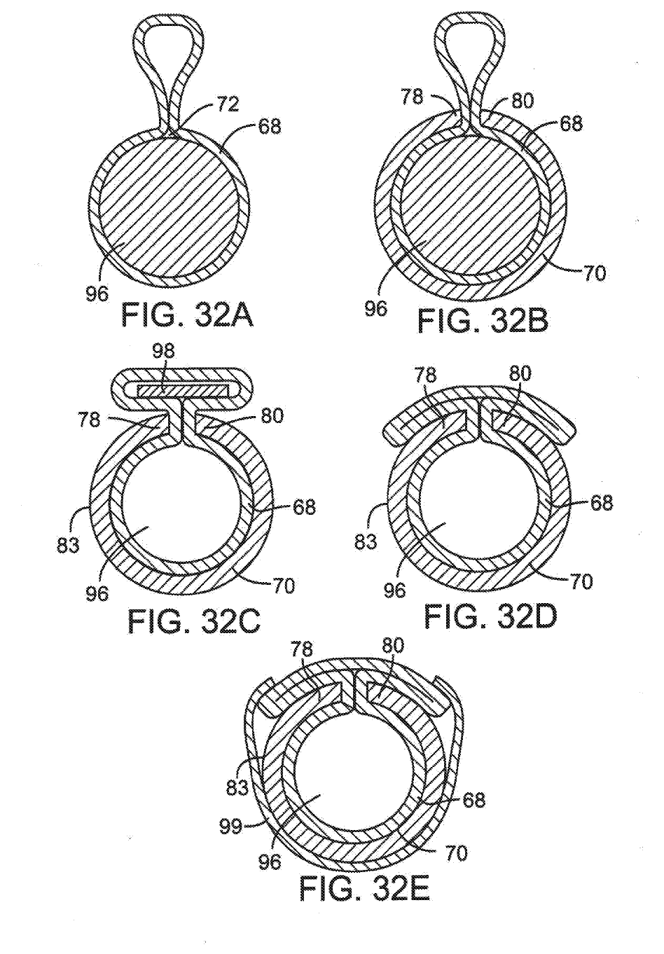

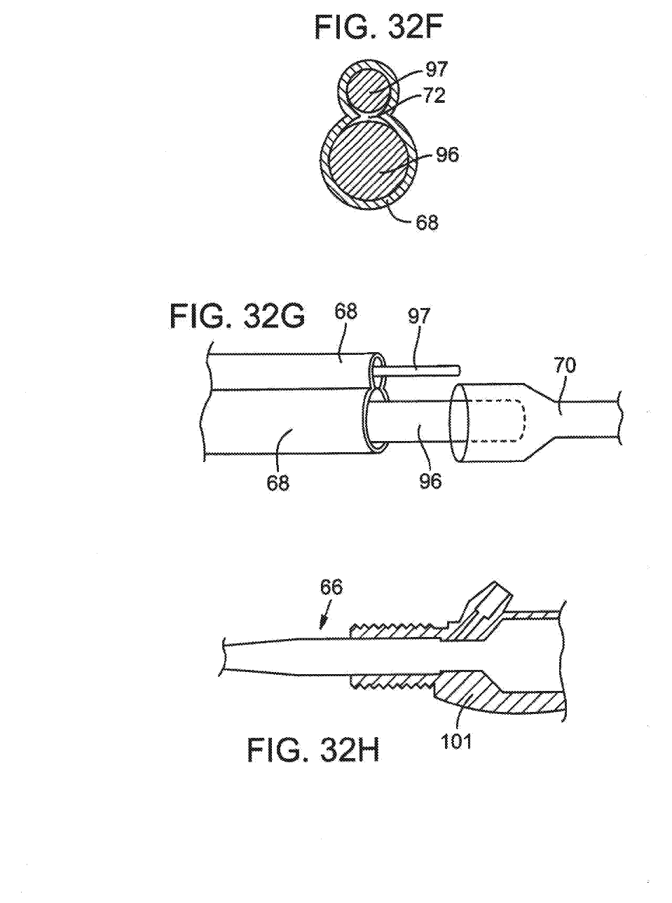

FIG. 30 illustrates a block diagram of one method of producing a sheath for use with a delivery apparatus in minimally invasive surgery. One or more mandrels can be provided (step 300). The mandrel can be provided with an exterior coating, such as a Teflon® coating, and the mandrel's diameter can be predetermined based on the desired size of the resulting sheath. A liner that will become the inner polymeric layer of the sheath, such as a PTFE or high density polyethylene liner, can be mounted on the mandrel (step 302). The liner can be etched and/or surface treated prior to being mounted on the mandrel, according to conventional etching and surface treatment methods. FIG. 32A illustrates a section view of a sheath at steps 300 and 302 of FIG. 30. A coated mandrel 96 is inserted within the lumen 72 of the inner polymeric layer 68. The circumference of the inner polymeric layer 68 is larger than the circumference of the mandrel 96, such that an excess portion of the inner polymeric layer 68 can be gathered above the mandrel 96.

A layer of material that will become the outer polymeric tubular layer, such as a layer comprising polyurethane or polyolefin, can be cut or notched through all, substantially all, or a part of the thickness of the layer (step 304). Such a cut or notch can extend longitudinally along the length of the layer and can extend along substantially the entire length of the outer polymeric tubular layer. In alternative aspects, the cut or notch can be provided along only a portion of the outer polymeric tubular layer. For example, the outer polymeric tubular layer can be cut starting at the distal end of the outer polymeric tubular layer, with the cut ending before the proximal end of the outer polymeric tubular layer. In one aspect, the cut can end at a transition, where the outer diameter of the outer polymeric tubular layer increases or decreases. In one specific aspect, the cut or notch can extend longitudinally along about 75% of the length of the sheath.

The cut or notched outer polymeric tubular layer can be applied, positioned, adhered, mounted, thermally fused or bonded, dip coated, and/or otherwise coupled to the etched inner liner (step 306). FIG. 32B shows a section view of the sheath at step 306 of FIG. 30, with outer polymeric tubular layer 70 applied to the inner polymeric layer 68 such that a portion of the inner polymeric layer 68 extends between the cut formed between first and second portions 78, 80 of the outer polymeric tubular layer 70.

In alternative aspects, the outer polymeric tubular layer can be notched or cut after being mounted on the inner liner/mandrel assembly. The outer polymeric tubular layer can optionally be provided with a hydrophilic coating and/or provided with additional layers, such as being dip coated with polyurethane. Some portion of the inner liner can protrude through the cut in the outer polymeric tubular layer after an outer polymeric tubular layer is mounted onto the inner liner/mandrel arrangement. Using, for example, a split tool, the protruding portion of the inner liner can be folded down onto the outer surface of the outer polymeric tubular layer (step 308). In some aspects, the protruding portion of the inner liner is folded down along the entire length of the resulting sheath, while in other aspects, the protruding portion of the inner liner is only present along a portion of the length of the sheath or is only folded down along a portion of the length of the resulting sheath. FIG. 32C shows a section view of the sheath at step 308 of FIG. 30. A split tool 98 is used to fold the excess portion of inner polymeric layer 68 over a portion of the outer surface 83 of the outer polymeric tubular layer 70. FIG. 32D shows a section view of the sheath after completion of step 308 of FIG. 30. Split tool 98 has been removed, and folding of the excess portion of the inner polymeric layer 68 has been completed. FIG. 32E shows a section view of an outer covering, such as outer polymeric covering 99, that can be applied such that it overlaps a portion of the folded portion of inner polymeric layer 68. The outer polymeric covering 99 contacts at least a portion of the outer surface 83 of the outer polymeric tubular layer 70.

A soft, atraumatic tip can be provided at the distal end of the resulting sheath (step 310). Additional outer layers can also be applied if desired. Then, a layer of heat shrink tubing, such as fluorinated ethylene propylene (FEP) heat shrink tubing, can be positioned over the entire assembly (step 312). An appropriate amount of heat is applied, thus shrinking the heat shrink tubing and compressing the layers of the sheath together, such that components of the sheath can be thermally bonded or fused together where desired. Once the components of the sheath have been bonded together, the heat shrink tubing can be removed (step 314). Finally, the proximal end of the sheath can be adhered or otherwise attached to a housing of a catheter assembly, and the sheath can be removed from the mandrel (step 316).

FIG. 31 illustrates a block diagram of an alternative aspect of a method of making a sheath. An inner liner, such as an etched PTFE tubing, can be applied to a tapered mandrel, such as a 16 Fr tapered mandrel, and trimmed to an appropriate length (step 200). A second mandrel, such as a 0.070 inches diameter mandrel, can be inserted in the lumen of the inner liner such that the mandrels are arranged side by side in the inner liner (step 202). FIG. 32F shows a section view of a sheath at steps 200 and 202 of FIG. 31. An inner liner or inner polymeric layer 68 is applied on a first tapered mandrel 96. A second mandrel 97 is inserted into the lumen 72 of the inner polymeric layer 68 created by the excess portion of the inner polymeric layer 68, as described.

A notched or cut outer polymeric tubular layer, such as high density polyethylene tubing that has been notched or cut longitudinally, can be slid onto the tapered mandrel and a portion of the inner liner, starting at the distal end of the tapered mandrel (step 204). The second mandrel can then be removed (step 206). FIG. 32G illustrates a perspective view of the sheath at steps 204 and 206 of FIG. 31. A polymeric outer tubular layer 70 having a longitudinal cut is applied over the tapered mandrel 96 and inner polymeric layer 68. The outer tubular layer conforms to the portion of the inner polymeric layer around the tapered mandrel 96, and the portion of the inner polymeric layer 68 around the second mandrel 97 extends through the longitudinal cut in the outer polymeric tubular layer 70.

A split tool can be inserted into the portion of the lumen of the inner liner that was previously occupied by the second mandrel (step 208). The split tool can then be used to form folds and/or pleats in the excess portion of the inner liner, which now extends through the longitudinal cut in the outer polymeric tubular layer (step 210). A radiopaque marker band can optionally be applied at the distal end of the sheath (step 212). Heat shrink tubing, such as FEP heat shrink tubing, can be applied over the entire sheath, and heat can be applied to compress the components of the sheath and bond or fuse them together (step 214). The split tool, heat shrink tubing, and second mandrel can then be removed (step 216). The sheath can then be utilized with a delivery apparatus, such as by bonding the proximal end of the sheath to a polycarbonate housing of a delivery apparatus or catheter assembly (step 218).

FIG. 32H illustrates an elevation view of the sheath at step 218 of FIG. 31. The sheath 66, made according to described methods and processes, can be attached or bonded to a housing 101, such as by bonding the proximal end of the sheath 66 to the polycarbonate housing 101.

In another example, disclosed expandable sheaths can be made using a reflowed mandrel process. A mandrel can be provided, with the size of the mandrel defining the inner diameter of the sheath lumen in its resting configuration. A tube of material, such as a PTFE tube that will become the sheath's inner liner, can be provided with an inner diameter greater than that of the mandrel (e.g., a 9 mm PTFE tube can be mounted on a 6 mm mandrel). The PTFE tube can be mounted on the mandrel and prepared into the final folded configuration by folding the excess material of the PTFE tube over to one or both sides. An HDPE tube that will serve as the outer layer can then be placed over the PTFE liner. The two-layer assembly can then be thermally fused together. For example, a reflow process can be performed where the assembly is heated to a temperature high enough such that the inner and/or outer layers are at least partially melted and are then fused together as the heat is removed and the assembly cools.

An elastic cover can be placed over at least part of the fused layers (e.g., over a proximal section of the sheath) and held in place using a thermal process. In some aspects, the same thermal process can bond the layers of the sheath and the elastic cover. In other aspects, a first thermal process can be used to fuse the layers of the sheath, and a second thermal process can be used to secure the elastic cover to the sheath. In some aspects, the elastic cover can be heat shrink tubing that is applied over the expandable sheath and heated to a temperature high enough to cause the tubing to shrink around the sheath. In some aspects, a distal soft tip can then be attached to the shaft of the expandable sheath.

In some aspects, the outer layer can be co-extruded with an adhesive layer, such as a layer formed from Tecoflex™, such that the Tecoflex™ is positioned on an inner surface of the outer layer—in this manner, the Tecoflex™ will be positioned between the inner and outer layers in the completed sheath. In these aspects, an HDPE tube can be provided with a coating of Tecoflex™ on the inner surface. The HDPE tube can be slit along the length of the tube to open and flatten it and then cut using a template in some aspects. For example, for specific applications, portions of the outer layer can be cut and removed using a template. The cut HDPE can then be placed on the inner liner on the mandrel. In some aspects, only a portion of the outer layer will have the adhesive Tecoflex™. In these aspects, the sections without Tecoflex™ will only be partially fused to the inner liner. In some aspects, the entire inner surface of the outer layer will have the Tecoflex™, and the inner surface of the outer layer can be positioned so that it contacts the inner liner on the mandrel. To position the inner and outer layers, as shown in the sheath of FIG. 39, the folded portion of the inner liner can be lifted up, and an edge of the outer layer can be tucked beneath the fold.

Sheaths of the present disclosure can be used with various methods of introducing a prosthetic device into a patient's vasculature. One such method comprises positioning an expandable sheath in a patient's vessel, passing a device through the introducer sheath, which causes a portion of the sheath surrounding the device to expand and accommodate the profile of the device, and automatically retracting the expanded portion of the sheath to its original size after the device has passed through the expanded portion. In some methods, the expandable sheath can be sutured to the patient's skin at the insertion site so that once the sheath is inserted the proper distance within the patient's vasculature, it does not move once the implantable device starts to travel through the sheath.

Disclosed aspects of an expandable sheath can be used with other delivery and minimally invasive surgical components, such as an introducer and loader. In one aspect, the expandable sheath can be flushed to purge any air within the sheath, using, for example, flush port 103 (FIG. 35). An introducer can be inserted into the expandable sheath, and the introducer/sheath combination can be fully inserted into vasculature over a guiding device, such as a 0.35″ guidewire. Preferably, the seam formed by the intersection of the folded portion of the inner liner and the overlapping portion of the outer layer can be positioned such it is oriented downward (posterior). Once the sheath and introducer are fully inserted into a patient's vasculature, in some aspects, the expandable sheath can be sutured in place at the insertion site. In this manner, the expandable sheath can be substantially prevented from moving once positioned within the patient.

The introducer can then be removed, and a medical device, such as a transcatheter heart valve, can be inserted into the sheath, in some instances, using a loader. Such methods can additionally comprise placing the tissue heart valve in a crimped state on the distal end portion of an elongated delivery apparatus and inserting the elongated delivery device with the crimped valve into and through the expandable sheath. Next, the delivery apparatus can be advanced through the patient's vasculature to the treatment site, where the valve can be implanted.

Typically, the medical device has a greater outer diameter than the diameter of the sheath in its original configuration. The medical device can be advanced through the expandable sheath towards the implantation site, and the expandable sheath can locally expand to accommodate the medical device as the device passes through. The radial force exerted by the medical device can be sufficient to locally expand the sheath to an expanded diameter (e.g., the expanded configuration) just in the area where the medical device is currently located. Once the medical device passes a particular location of the sheath, the sheath can at least partially contract to the smaller diameter of its original configuration. The expandable sheath can thus be expanded without the use of inflatable balloons or other dilators. Once the medical device is implanted, the sheath and any sutures holding in place can be removed. In some aspects, it is preferable to remove the sheath without rotating it.

### EXEMPLARY ASPECTS

EXAMPLE 1: A sheath for delivering a medical device, wherein the sheath has a proximal and a distal end and comprises: an expandable inner liner having an inner surface and an outer surface, wherein the inner surface of the expandable inner liner defines a lumen and wherein the inner liner comprises at least one folded portion having an inner portion and outer portion; an outer layer having an inner surface and an outer surface and extending at least partially around the inner liner such that a first portion of the outer surface of the outer layer is positioned adjacent to the inner portion of the at least one folded portion of the inner liner, while a first portion of the inner surface of the outer layer is positioned adjacent to the outer portion of the at least one folded portion of the inner liner; and wherein the inner liner comprises at least one first portion and at least one second portion, wherein an outer surface of the at least one first portion comprises a composition and/or morphology that is substantially different from an outer surface of the at least one second portion; and wherein an outwardly directed radial force from a prosthetic device moving through the inner lumen unfolds the at least one folded portion to allow expansion of the sheath.

EXAMPLE 2: The sheath of any examples herein, particularly example 1, wherein the at least one first portion of the inner liner is etched.

EXAMPLE 3: The sheath of any examples herein, particularly example 1 or 2, wherein the at least one second portion of the inner liner is unetched.

EXAMPLE 4: The sheath of any examples herein, particularly example 1 or 2, wherein the at least one second portion is etched and subsequently surface-modified such that a composition of the outer surface of the at least one second portion is substantially different from a composition of the outer surface of the at least one first portion.

EXAMPLE 5: The sheath of any examples herein, particularly examples 1-4, wherein the outer surface of the at least one second portion comprises a morphology that is substantially different from a morphology of the outer surface of the at least one first portion.

EXAMPLE 6: The sheath of any examples herein, particularly examples 1-5, wherein the inner liner comprises a fluorocarbon-based polymer.

EXAMPLE 7: The sheath of any examples herein, particularly examples 1-6, wherein the inner liner comprises polytetrafluoroethylene (PTFE).

EXAMPLE 8: The sheath of any examples herein, particularly examples 6-7, wherein the outer surface of the at least one first portion comprises a fluorine-to-carbon ratio that is lower than a fluorine-to-carbon ratio present on the outer surface of the at least one second portion of the inner liner.

EXAMPLE 9: The sheath of any examples herein, particularly examples 1-8, wherein the outer surface of the at least one first portion of the inner liner exhibits an RMS value lower than an RMS value of the outer surface of the at least one second portion of the inner liner.

EXAMPLE 10: The sheath of any examples herein, particularly examples 4-9, wherein the at least one second portion of the inner liner is surface-modified by a laser ablation.

EXAMPLE 11: The sheath of any examples herein, particularly examples 4-10, wherein the at least one second portion is surface-modified and comprises a gradient morphology.

EXAMPLE 12: The sheath of any examples herein, particularly example 11, wherein the gradient morphology has a predetermined pattern.

EXAMPLE 13: The sheath of any examples herein, particularly examples 1-12, wherein the at least one second portion extends longitudinally along a length of the inner liner.

EXAMPLE 14: The sheath of any examples herein, particularly examples 1-13, wherein the at least one second portion extends circumferentially around a circumference of the inner liner.

EXAMPLE 15: The sheath of any examples herein, particularly examples 1-14, wherein the inner liner comprises two or more first portions.

EXAMPLE 16: The sheath of any examples herein, particularly examples 1-15, wherein the inner liner comprises two or more second portions.

EXAMPLE 17: The sheath of any examples herein, particularly example 16, wherein each of the two or more second portions comprises the same or different composition and/or morphology.

EXAMPLE 18: The sheath of any examples herein, particularly example 16 or 17, wherein each of the two or more second portions has a length that is the same or different.

EXAMPLE 19: The sheath of any examples herein, particularly examples 1-18, wherein the first portion of the inner liner is followed by the second portion of the inner liner.

EXAMPLE 20: The sheath of any examples herein, particularly examples 1-19, wherein the second portion of the inner liner is followed by the first portion of the inner liner.

EXAMPLE 21: The sheath of any examples herein, particularly examples 1-20, wherein the at least one first portion has a length different from a length of the at least one second portion.

EXAMPLE 22: The sheath of any examples herein, particularly example 21, wherein the length of the at least one first portion is greater than the length of the at least one second portion.

EXAMPLE 23: The sheath of any examples herein, particularly examples 1-22, wherein the inner liner has a wall thickness ranging between about 0.002 inches and about 0.006 inches.

EXAMPLE 24: The sheath of any examples herein, particularly example 23, wherein the at least one first portion has a wall thickness different from a wall thickness of the at least one second portion.

EXAMPLE 25: The sheath of any examples herein, particularly example 24, wherein the wall thickness of the at least one second portion is up to about 2% larger than the wall thickness of the at least one first portion.

EXAMPLE 26: The sheath of any examples herein, particularly example 24, wherein the wall thickness of the at least one second portion is up to about 2% smaller than the wall of the at least one first portion.

EXAMPLE 27: The sheath of any examples herein, particularly examples 25-26, wherein when two or more second portions are present, each of the two or more second portions comprises a wall thickness that is the same or different.

EXAMPLE 28: The sheath of any examples herein, particularly examples 1-27, wherein the at least one second portion of the inner liner extends along the inner portion and/or the outer portion of the at least one folded portion of the inner liner.

EXAMPLE 29: The sheath of any examples herein, particularly examples 1-28, wherein the at least one second portion of the inner liner extends along portions that contact the outer surface of the outer layer.

EXAMPLE 30: The sheath of any examples herein, particularly examples 1-29, wherein the outer layer comprises high-density polyethylene polymer (HDPE), nylon, or polypropylene.

EXAMPLE 31: The sheath of any examples herein, particularly examples 1-30, wherein the at least one second portion of the inner liner exhibits substantially less stickiness to the outer layer as compared to the stickiness of the at least one first portion of the inner liner to the outer layer.

EXAMPLE 32: The sheath of any examples herein, particularly examples 1-31, wherein the outer surface of the at least one second portion exhibits a contact angle from greater than about 65° to about 140°.

EXAMPLE 33: The sheath of any examples herein, particularly examples 1-32, wherein the outer surface of the at least one first portion exhibits a contact angle from about 30° to about 90°.

EXAMPLE 34: The sheath of any examples herein, particularly examples 1-33, wherein the inner liner comprises the at least one second portion at the proximal end of the sheath.

EXAMPLE 35: The sheath of any examples herein, particularly examples 1-33, wherein the second portion of the inner liner is separated from the proximal end of the sheath by the first portion of the inner liner.

EXAMPLE 36: The sheath of any examples herein, particularly example 35, wherein the length of the first portion is substantially similar or shorter than the length of the second portion.

EXAMPLE 37: The sheath of any examples herein, particularly example 35 or 36, wherein the second portion of the inner liner is followed by an additional first portion of the inner liner.

EXAMPLE 38: The sheath of any examples herein, particularly example 37, wherein the length of the second portion of the inner liner is shorter than a length of the additional first portion of the inner liner.

EXAMPLE 39: The sheath of any examples herein, particularly examples 1-38, wherein the sheath further comprises an outer jacket comprising an inner surface and outer surface and a proximal end and a distal end and extending at least partially around the outer layer such that the inner surface of the outer jacket overlies the outer surface of the outer layer.

EXAMPLE 40: The sheath of any examples herein, particularly example 39, wherein the outer jacket extends from the proximal end of the sheath along at least a portion of a length of the sheath.

EXAMPLE 41: The sheath of any examples herein, particularly example 39 or 40, wherein the outer jacket has a first predetermined length.

EXAMPLE 42: The sheath of any examples herein, particularly example 41, wherein the second portion extends from the proximal end of the sheath, and wherein the length of the at least one second portion is substantially similar to the first predetermined length of the outer jacket.

EXAMPLE 43: The sheath of any examples herein, particularly example 42, wherein the second portion extends from the proximal end of the sheath, and wherein the length of the at least one second portion is larger than the first predetermined length of the outer jacket.

EXAMPLE 44: The sheath of any examples herein, particularly example 41, wherein the second portion of the inner liner is separated from the proximal end of the sheath by the first portion, and wherein a total length of the first portion of the inner liner and the second portion of the inner liner is substantially similar to the first predetermined length of the outer jacket.

EXAMPLE 45: The sheath of any examples herein, particularly example 41, wherein the second portion of the inner liner is separated from the proximal end of the sheath by the first portion of the inner liner, and wherein a total length of the first portion of the inner liner and the second portion of the inner liner is larger than the first predetermined length of the outer jacket.

EXAMPLE 46: The sheath of any examples herein, particularly examples 39-45, wherein the distal end of the outer jacket is substantially sealed with the outer layer of the sheath.

EXAMPLE 47: The sheath of any examples herein, particularly examples 39-46, wherein the outer jacket extends from the proximal end of the sheath to the distal end of the sheath.

EXAMPLE 48: The sheath of any examples herein, particularly example 47, wherein the at least one second portion of the inner liner extends along the inner portion and/or the outer portion of the at least one folded portion of the inner liner.

EXAMPLE 49: The sheath of any examples herein, particularly examples 47 or 48, wherein the at least one second portion of the inner liner extends along portions of the inner liner that contact the outer surface of the outer layer.

EXAMPLE 50: The sheath of any examples herein, particularly examples 1-49, wherein the inner liner comprises two or more folded portions.

EXAMPLE 51: The sheath of any examples herein, particularly example 50, wherein the at least one second portion of the inner liner extends along the inner portion and/or the outer portion of each of the two or more folded portions.

EXAMPLE 52: The sheath of any examples herein, particularly examples 1-51, wherein the sheath exhibits a push force upon passing of the prosthetic device of at least about 10% lower than a substantially identical reference sheath that does not comprise the second portion.

EXAMPLE 53: The sheath of any examples herein, particularly examples 1-52, wherein the sheath exhibits a push force upon passing of the prosthetic device of less than about 50 N.

EXAMPLE 54: A method of manufacturing a sheath for delivering a medical device, the method comprising: providing an inner liner having an inner surface and an outer surface, wherein the inner surface defines a lumen therethrough, wherein the inner liner comprises at least one first portion and at least one second portion, wherein an outer surface of the at least one first portion comprises a composition and/or morphology that is substantially different from an outer surface of the at least one second portion; and wherein the inner liner comprises at least one folded portion having an inner portion and outer portion; providing an outer layer having an inner surface and an outer surface such that it extends at least partially around the inner liner such that at least a first portion of the outer surface of the outer layer is positioned adjacent to the inner portion of the at least one folded portion of the inner liner, while a first portion of the inner surface of the outer layer is positioned adjacent to the outer portion of the at least one folded portion of the inner liner; and forming an expandable sheath that is configured to expand when an outwardly directed radial force from a prosthetic device moving through the inner lumen unfolds the at least one folded portion.

EXAMPLE 55: The method of any examples herein, particularly example 54, wherein the at least one first portion and at least one second portion are formed by a selective etching.

EXAMPLE 56: The method of any examples herein, particularly example 55, wherein the selective etching is performed around the circumference, linearly along a length of the sheath, or a combination thereof.

EXAMPLE 57: The method of any examples herein, particularly example 55 or 56, wherein the at least one first portion is formed by masking a portion of the inner liner to form a masked portion and then etching remaining unmasked portions of the inner liner with an etchant.

EXAMPLE 58: The method of any examples herein, particularly example 57, further comprising a step of forming the at least one second portion by unmasking the masked portion after the etching.

EXAMPLE 59: The method of any examples herein, particularly examples 54-58, wherein the inner liner comprises a fluorocarbon-based polymer.

EXAMPLE 60: The method of any examples herein, particularly examples 54-59, wherein the inner liner comprises a polytetrafluoroethylene (PTFE).

EXAMPLE 61: The method of any one of any examples herein, particularly example 57-60, wherein the etchant comprises a liquid, a fluid, a gas, a plasma, or a combination thereof.

EXAMPLE 62: The method of any examples herein, particularly examples 57-61, wherein the etchant comprises a sodium naphthalene solution.

EXAMPLE 63: The method of any examples herein, particularly examples 57-62, wherein the etchant comprises an oxygen plasma, hydrogen plasma, nitrogen plasma, argon plasma, or any combination thereof.

EXAMPLE 64: The method of any examples herein, particularly examples 55 or 56, wherein the at least one first portion is formed by etching at least a portion of the inner liner to form an etched inner liner.

EXAMPLE 65: The method of any examples herein, particularly example 64, further comprising ablating at least a portion of the etched inner liner, thereby forming the at least one second portion of the inner liner.

EXAMPLE 66: The method of any examples herein, particularly example 65, wherein the step of ablating comprises forming a gradient having a predetermined pattern.

EXAMPLE 67: The method of any examples herein, particularly examples 54-66, wherein the outer surface of the at least one second portion of the inner liner comprises a composition that is substantially different from a composition of the outer surface of the at least one first portion of the inner liner.

EXAMPLE 68: The method of any examples herein, particularly examples 54-67, wherein the outer surface of the at least one second portion comprises a morphology that is substantially different from a morphology of the outer surface of the at least one second portion.

EXAMPLE 69: The method of any examples herein, particularly examples 54-63, wherein the outer surface of the at least one first portion comprises a fluorine-to-carbon ratio that is lower than a fluorine-to-carbon ratio present on the outer surface of the at least one second portion of the inner liner.

EXAMPLE 70: The method of any examples herein, particularly examples 65-69, wherein the outer surface of the at least one first portion of the inner liner exhibits an RMS value lower than an RMS value of the outer surface of the at least one second portion of the inner liner.

EXAMPLE 71: The method of any examples herein, particularly examples 54-70, wherein the at least one second portion extends longitudinally along a length of the inner liner.

EXAMPLE 72: The method of any examples herein, particularly examples 54-71, wherein the at least one second portion extends circumferentially around a circumference of the inner liner.

EXAMPLE 73: The method of any examples herein, particularly examples 54-72, wherein the inner liner comprises two or more first portions.

EXAMPLE 74: The method of any examples herein, particularly examples 54-73, wherein the inner liner comprises two or more second portions.

EXAMPLE 75: The method of any examples herein, particularly example 74, wherein each of the two or more second portions comprises the same or different composition or morphology.

EXAMPLE 76: The method of any examples herein, particularly example 74 or 75, wherein each of the two or more second portions has a length that is the same or different.

EXAMPLE 77: The method of any examples herein, particularly examples 74-76, wherein the first portion of the inner liner is followed by the second portion of the inner liner.

EXAMPLE 78: The method of any examples herein, particularly examples 74-77, wherein the second portion of the inner liner is followed by the first portion of the inner liner.

EXAMPLE 79: The method of any examples herein, particularly examples 74-78, wherein the at least one first portion has a length different from a length of the at least one second portion.

EXAMPLE 80: The method of any examples herein, particularly example 79, wherein the length of the at least one first portion is greater than the length of the at least one second portion.

EXAMPLE 81: The method of any examples herein, particularly examples 54-80, wherein the inner liner has a wall thickness ranging between about 0.002 inches and about 0.006 inches.

EXAMPLE 82: The method of any examples herein, particularly example 81, wherein the at least one first portion has a wall thickness different from a wall thickness of the at least one second portion.

EXAMPLE 83: The method of any examples herein, particularly example 82, wherein the wall thickness of the at least one second portion is up to about 2% larger than the wall thickness of the at least one first portion.

EXAMPLE 84: The method of any examples herein, particularly example 82, wherein the wall thickness of the at least one second portion is up to about 2% smaller than the wall of the at least one first portion.

EXAMPLE 85: The method of any examples herein, particularly examples 83 or 84, wherein when two or more second portions are present, each of the two or more second portions comprises a wall thickness that is the same or different.

EXAMPLE 86: The method of any examples herein, particularly examples 54-85, wherein the at least one second portion of the inner liner extends along the inner portion and/or the outer portion of the at least one folded portion of the inner liner.

EXAMPLE 87: The method of any examples herein, particularly examples 54-86, wherein the at least one second portion of the inner liner extends along portions of the inner liner that contact the outer surface of the outer layer.

EXAMPLE 88: The method of any examples herein, particularly examples 54-87, wherein the outer layer comprises high-density polyethylene polymer (HDPE), nylon, or polypropylene.

EXAMPLE 89: The method of any examples herein, particularly examples 54-88, wherein the at least one second portion of the inner liner exhibits substantially less stickiness to the outer layer as compared to the stickiness of the at least one first portion of the inner liner to the outer layer.

EXAMPLE 90: The method of any examples herein, particularly examples 54-89, wherein the outer surface of the at least one second portion exhibits a contact angle from greater than about 65° to about 140°.

EXAMPLE 91: The method of any examples herein, particularly examples 54-90 wherein the outer surface of the at least one first portion exhibits a contact angle from about 30° to about 90°.

EXAMPLE 92: The method of any examples herein, particularly examples 54-91, wherein the inner liner comprises the at least one second portion of the inner liner at the proximal end of the sheath.

EXAMPLE 93: The method of any examples herein, particularly examples 54-92, wherein the second portion of the inner liner is separated from the proximal end of the sheath by the first portion of the inner liner.

EXAMPLE 94: The method of any examples herein, particularly example 93, wherein the length of the first portion is substantially similar or shorter than the length of the second portion.

EXAMPLE 95: The method of any examples herein, particularly example 93 or 94, wherein the second portion of the inner liner is followed by an additional first portion of the inner liner.

EXAMPLE 96: The method of any examples herein, particularly example 95, wherein the length of the second portion of the inner liner is shorter than a length of the additional first portion of the inner liner.

EXAMPLE 97: The method of any examples herein, particularly examples 54-96, further comprises providing an outer jacket comprising an inner surface and outer surface and a proximal end and a distal end such that the outer jacket extends at least partially around the outer layer such that the inner surface of the outer jacket overlies the outer surface of the outer layer.

EXAMPLE 98: The method of any examples herein, particularly example 97, wherein the outer jacket extends from the proximal end of the sheath along at least a portion of a length of the sheath.

EXAMPLE 99: The method of any examples herein, particularly examples 97-98, wherein the outer jacket has a first predetermined length.

EXAMPLE 100: The method of any examples herein, particularly example 98 or 99, wherein the second portion extends from the proximal end of the sheath, and wherein the length of the at least one second portion is substantially similar to the first predetermined length of the outer jacket.

EXAMPLE 101: The method of any examples herein, particularly example 98 or 99, wherein the second portion extends from the proximal end of the sheath and wherein the length of the at least one second portion is larger than the first predetermined length of the outer jacket.

EXAMPLE 102: The method of any examples herein, particularly example 98 or 99, wherein the second portion of the inner liner is separated from the proximal end of the sheath by the first portion of the inner liner, and wherein a total length of the first portion of the inner liner and the second portion of the inner liner is substantially similar to the first predetermined length of the outer jacket.

EXAMPLE 103: The method of any examples herein, particularly example 98 or 99, wherein the second portion of the inner liner is separated from the proximal end of the sheath by the first portion of the inner liner, and wherein a total length of the first portion of the inner liner and the second portion of the inner liner is larger than the first predetermined length of the outer jacket.

EXAMPLE 104: The method of any examples herein, particularly examples 96-103, wherein the distal end of the outer jacket is substantially sealed with the outer layer of the sheath.

EXAMPLE 105: The method of any examples herein, particularly examples 96-104, wherein the outer jacket extends from the proximal end of the sheath to the distal end of the sheath.

EXAMPLE 106: The method of any examples herein, particularly example 105, wherein the at least one second portion of the inner liner extends along the inner portion and/or the outer portion of the at least one folded portion of the inner liner.

EXAMPLE 107: The method of any examples herein, particularly examples 105 or 106, wherein the at least one second portion of the inner liner extends along portions of the inner liner that contact the outer surface of the outer layer.

EXAMPLE 108: The method of any examples herein, particularly examples 54-107, wherein the inner liner comprises two or more folded portions.

EXAMPLE 109: The method of any examples herein, particularly example 107, wherein the at least one second portion of the inner liner extends along the inner portion and/or the outer portion of each of the two or more folded portions.

EXAMPLE 110: The method of any examples herein, particularly examples 54-109, wherein the sheath exhibits a push force upon passing of the prosthetic device of at least about 10% lower than a substantially identical reference sheath that does not comprise the second portion.

EXAMPLE 111: The method of any examples herein, particularly examples 54-110, wherein the sheath exhibits a push force upon passing of the prosthetic device of less than about 50 N.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A sheath for delivering a medical device, wherein the sheath has a proximal and a distal end and comprises:
an expandable inner layer comprising an inner surface, an outer surface, and a longitudinal cut extending along at least a portion of the length of the sheath;
an outer tubular layer having an inner surface and an outer surface, the outer tubular layer extending at least partially around the outer surface of the inner layer;
a tie layer positioned between at least a portion of the expandable inner layer and the outer tubular layer, thereby adhering the expandable inner layer to the outer tubular layer; and
a strain relief section positioned at a proximal end of the sheath; and
wherein an outwardly directed radial force from a prosthetic device moving through an inner lumen of the sheath expands the sheath.

2. The sheath of claim 1, wherein the strain relief section is tapered.

3. The sheath of claim 2, wherein the strain relief section include a flared proximal end portion, the taper of the strain relief section is located between a distal end of the strain relief section and the flared proximal end.

4. The sheath of claim 1, wherein the strain relief section extends from a proximal end of the sheath and has a Durometer and/or stiffness greater than a corresponding Durometer and stiffness than the outer tubular layer.

5. The sheath of claim 1, wherein the longitudinal cut extends through the entire thickness of the inner layer.

6. The sheath of claim 1, wherein the longitudinal cut extends along the entire length of the sheath.

7. The sheath of claim 1, wherein the inner layer comprises a first portion and a second portion, and the second portion at least partially overlaps the first portion in a partial coil configuration.

8. The sheath of claim 7, wherein the outwardly directed radial force from a prosthetic device moving through the inner lumen of the sheath expands the sheath causing the first portion and the second portion to slide circumferentially with respect to each other allowing a diameter of the inner lumen of the sheath to enlarge.

9. The sheath of claim 7, wherein the outwardly directed radial force from a prosthetic device moving through the inner lumen of the sheath expands the sheath to form a gap between a longitudinal edge of the first portion and a longitudinal edge of the second portion.

10. The sheath of claim 1, wherein the outer tubular layer includes a continuous layer covering the outer surface of the inner layer.

11. The sheath of claim 1, further comprising a lubricant between the inner layer and outer tubular layer.

12. The sheath of claim 1, wherein the tie layer comprises at least one of a polyolefin and a polyamide.

13. The sheath of claim 1, wherein the tie layer has a Shore A hardness of less than 90A.

14. The sheath of claim 11, wherein the lubricant comprises a medical-grade lubricant.

15. The sheath of claim 11, wherein the lubricant comprises a silicone.

16. The sheath of claim 11, wherein the lubricant comprises a PTFE lubricant.

17. The sheath of claim 1, wherein upon removal of the outwardly directed radial force from a prosthetic device moving through the inner lumen of the sheath, at least one of the inner layer or the outer tubular layer return toward an unexpanded configuration.
